SEQUENCE LISTING (44 sequences)

SEQ ID NO: 1 - DNA, 512 bp, Artificial Sequence
  1-IREScrTMV
gaattcgtcgattcggttgcagcatttaaagcggttgacaactttaaaagaaggaaaaagaaggttgaagaaaagggtgtagtaagtaagtataagtacagaccggagaagtacgccggtcctgattcgtttaatttgaaagaagaaaatgcctgctgcgaagagggtgaaattggatccggcggccaaacgagtcaaacttgatccagctgctaagcgagtgaagctagacggtggtggggggtctgggggaggtggtagcggaggtggaggtagcagagaccacatggtattgcacgaatatgtaaatgcggctggcattaccggaggggggggtagcgggggcggcggatccggtggtggaagcagcagagaccatatggttttgcacgagtatgtcaatgccgcgggcataacgtaataaaaaaaaaaaaaaaaaaaaaaaaaaaaaaaaaaaaaaaaaaaaaaaaaaaacaccaccaccaccaccactgcatggttaattcctcctg

SEQ ID NO: 2 - DNA, 858 bp, Artificial Sequence
  1-IRESNtHSF
ggcacgaggctcccattaatatttcttcttctgtgtaattccattattctgtagtagattcacgtccgagtttaaagaagagagaaaactgaaaaggcagaaaattccagagctttagatttagccaaagatagttatggtcgtgttgttcttggtgaagattggcaaagtaggagccaatggaagaaactaagatcataatcaatcgccccaaaaacaaccttgttcattctatggtttttctcttcggtttctatgtttgggattgggaattcctcactgtccttttgcttttcagttattgctccttctaattttccctagctaggatcttctcaattaatttcctttttcattttcaactaactcataattagcccaaatcttcaaaagagttttgtgtaagttgatagacgtttagagaaacagagaaatacaggggaaaaacaagggatgcctgctgcgaagagggtgaaattggatccggcggccaaacgagtcaaacttgatccagctgctaagcgagtgaagctagacggtggtggggggtctgggggaggtggtagcggaggtggaggtagcagagaccacatggtattgcacgaatatgtaaatgcggctggcattaccggaggggggggtagcgggggcggcggatccggtggtggaagcagcagagaccatatggttttgcacgagtatgtcaatgccgcgggcataacgtaataaaaaaaaaaaaaaaaaaaaaaaaaaaaaaaaaaaaaaaaaaaaaaaaaaaacaccaccaccaccaccactgcatggttaattcctcctgcagataaaaaaaatccttagctttcgctaaggatgatttct

SEQ ID NO: 3 - DNA, 597 bp, Artificial Sequence
  1-IRESTuMV
gggaaagcttgcatgcctgcaggtcgactctagaaaaatataaaaactcaacacaacatacacaaaacgattaaagcaaacacaatctttcaaagcattcaaagcattcaagcaatcaaagattttcaaatcttttgtcgttatcaaagcaatcaccaacaggatccaggatccccgggtggtcagtcccttatgcctgctgcgaagagggtgaaattggatccggcggccaaacgagtcaaacttgatccagctgctaagcgagtgaagctagacggtggtggggggtctgggggaggtggtagcggaggtggaggtagcagagaccacatggtattgcacgaatatgtaaatgcggctggcattaccggaggggggggtagcgggggcggcggatccggtggtggaagcagcagagaccatatggttttgcacgagtatgtcaatgccgcgggcataacgtaataaaaaaaaaaaaaaaaaaaaaaaaaaaaaaaaaaaaaaaaaaaaaaaaaaaacaccaccaccaccaccactgcatggttaattcctcctgcagataaaaaaaatccttagctttcgctaaggatgatttct

SEQ ID NO: 4 - DNA, 554 bp, Artificial Sequence
  1-IREScrTEV
aaataacaaatctcaacacaacatatacaaaacaaacgaatctcaagcaatcaagcattctacttctattgcagcaatttaaatcatttcttttaaagcaaaagcaattttctgaaaattttcaccatttacgaacgatagcaatgcctgctgcgaagagggtgaaattggatccggcggccaaacgagtcaaacttgatccagctgctaagcgagtgaagctagacggtggtggggggtctgggggaggtggtagcggaggtggaggtagcagagaccacatggtattgcacgaatatgtaaatgcggctggcattaccggaggggggggtagcgggggcggcggatccggtggtggaagcagcagagaccatatggttttgcacgagtatgtcaatgccgcgggcataacgtaataaaaaaaaaaaaaaaaaaaaaaaaaaaaaaaaaaaaaaaaaaaaaaaaaaaacaccaccaccaccaccactgcatggttaattcctcctgcagataaaaaaaatccttagctttcgctaaggatgatttctgatatc

SEQ ID NO: 5 - DNA, 524 bp, Artificial Sequence
  1-CITESNTV
aaaaaaaaaaaagtaaagacaggaaactttactgactaacatgcctgctgcgaagagggtgaaattggatccggcggccaaacgagtcaaacttgatccagctgctaagcgagtgaagctagacggtggtggggggtctgggggaggtggtagcggaggtggaggtagcagagaccacatggtattgcacgaatatgtaaatgcggctggcattaccggaggggggggtagcgggggcggcggatccggtggtggaagcagcagagaccatatggttttgcacgagtatgtcaatgccgcgggcataacgtaataaaaaaaaaaaaaaaaaaaaaaaaaaaaaaaaaaaaaaaaaaaaaaaaaaaatcccagaggttcacaatgttagtgatggggcgctgaaagatgcgtagctacccttctggagccacttcctggtggtaagcagaaatccaagggtacggtggtacggtggaaagcagtccccaccaccaccaccaccactgcatggttaattcctcctg

SEQ ID NO: 6 - DNA, 550 bp, Artificial Sequence
  2-IREScrTMV (hairpin)
caggaggaattaaccatgcagtggtggtggtggtggtggaattcgtcgattcggttgcagcatttaaagcggttgacaactttaaaagaaggaaaaagaaggttgaagaaaagggtgtagtaagtaagtataagtacagaccggagaagtacgccggtcctgattcgtttaatttgaaagaagaaaatgcctgctgcgaagagggtgaaattggatccggcggccaaacgagtcaaacttgatccagctgctaagcgagtgaagctagacggtggtggggggtctgggggaggtggtagcggaggtggaggtagcagagaccacatggtattgcacgaatatgtaaatgcggctggcattaccggaggggggggtagcgggggcggcggatccggtggtggaagcagcagagaccatatggttttgcacgagtatgtcaatgccgcgggcataacgtaataaaaaaaaaaaaaaaaaaaaaaaaaaaaaaaaaaaaaaaaaaaaaaaaaaaacaccaccaccaccaccactgcatggttaattcctcctg

SEQ ID NO: 7 - DNA, 896 bp, Artificial Sequence
  2-IRESNtHSF (hairpin)
caggaggaattaaccatgcagtggtggtggtggtggtgggcacgaggctcccattaatatttcttcttctgtgtaattccattattctgtagtagattcacgtccgagtttaaagaagagagaaaactgaaaaggcagaaaattccagagctttagatttagccaaagatagttatggtcgtgttgttcttggtgaagattggcaaagtaggagccaatggaagaaactaagatcataatcaatcgccccaaaaacaaccttgttcattctatggtttttctcttcggtttctatgtttgggattgggaattcctcactgtccttttgcttttcagttattgctccttctaattttccctagctaggatcttctcaattaatttcctttttcattttcaactaactcataattagcccaaatcttcaaaagagttttgtgtaagttgatagacgtttagagaaacagagaaatacaggggaaaaacaagggatgcctgctgcgaagagggtgaaattggatccggcggccaaacgagtcaaacttgatccagctgctaagcgagtgaagctagacggtggtggggggtctgggggaggtggtagcggaggtggaggtagcagagaccacatggtattgcacgaatatgtaaatgcggctggcattaccggaggggggggtagcgggggcggcggatccggtggtggaagcagcagagaccatatggttttgcacgagtatgtcaatgccgcgggcataacgtaataaaaaaaaaaaaaaaaaaaaaaaaaaaaaaaaaaaaaaaaaaaaaaaaaaaacaccaccaccaccaccactgcatggttaattcctcctgcagataaaaaaaatccttagctttcgctaaggatgatttct

SEQ ID NO: 8 - DNA, 592 bp, Artificial Sequence
  2-IREScrTEV (hairpin)
caggaggaattaaccatgcagtggtggtggtggtggtgaaataacaaatctcaacacaacatatacaaaacaaacgaatctcaagcaatcaagcattctacttctattgcagcaatttaaatcatttcttttaaagcaaaagcaattttctgaaaattttcaccatttacgaacgatagcaatgcctgctgcgaagagggtgaaattggatccggcggccaaacgagtcaaacttgatccagctgctaagcgagtgaagctagacggtggtggggggtctgggggaggtggtagcggaggtggaggtagcagagaccacatggtattgcacgaatatgtaaatgcggctggcattaccggaggggggggtagcgggggcggcggatccggtggtggaagcagcagagaccatatggttttgcacgagtatgtcaatgccgcgggcataacgtaataaaaaaaaaaaaaaaaaaaaaaaaaaaaaaaaaaaaaaaaaaaaaaaaaaaacaccaccaccaccaccactgcatggttaattcctcctgcagataaaaaaaatccttagctttcgctaaggatgatttctgatatc

SEQ ID NO: 9 - DNA, 635 bp, Artificial Sequence
  2-IRESTuMV (hairpin)
caggaggaattaaccatgcagtggtggtggtggtggtggggaaagcttgcatgcctgcaggtcgactctagaaaaatataaaaactcaacacaacatacacaaaacgattaaagcaaacacaatctttcaaagcattcaaagcattcaagcaatcaaagattttcaaatcttttgtcgttatcaaagcaatcaccaacaggatccaggatccccgggtggtcagtcccttatgcctgctgcgaagagggtgaaattggatccggcggccaaacgagtcaaacttgatccagctgctaagcgagtgaagctagacggtggtggggggtctgggggaggtggtagcggaggtggaggtagcagagaccacatggtattgcacgaatatgtaaatgcggctggcattaccggaggggggggtagcgggggcggcggatccggtggtggaagcagcagagaccatatggttttgcacgagtatgtcaatgccgcgggcataacgtaataaaaaaaaaaaaaaaaaaaaaaaaaaaaaaaaaaaaaaaaaaaaaaaaaaaacaccaccaccaccaccactgcatggttaattcctcctgcagataaaaaaaatccttagctttcgctaaggatgatttct

SEQ ID NO: 10 - DNA, 562 bp, Artificial Sequence
  2-CITESNTV (hairpin)
caggaggaattaaccatgcagtggtggtggtggtggtgaaaaaaaaaaaagtaaagacaggaaactttactgactaacatgcctgctgcgaagagggtgaaattggatccggcggccaaacgagtcaaacttgatccagctgctaagcgagtgaagctagacggtggtggggggtctgggggaggtggtagcggaggtggaggtagcagagaccacatggtattgcacgaatatgtaaatgcggctggcattaccggaggggggggtagcgggggcggcggatccggtggtggaagcagcagagaccatatggttttgcacgagtatgtcaatgccgcgggcataacgtaataaaaaaaaaaaaaaaaaaaaaaaaaaaaaaaaaaaaaaaaaaaaaaaaaaaatcccagaggttcacaatgttagtgatggggcgctgaaagatgcgtagctacccttctggagccacttcctggtggtaagcagaaatccaagggtacggtggtacggtggaaagcagtccccaccaccaccaccaccactgcatggttaattcctcctg

SEQ ID NO: 11 - protein, 21 aa, E. Coli
MKKTAIAIAVALAGFATVAQA

SEQ ID NO: 12 - protein, 22 aa, Erwinia carotovora
MKYLLPTAAAGLLLLAAQPAMA

SEQ ID NO: 13 - protein, 23 aa, bacteria
MKKNIAFLLASMFVFSIATNAYA

SEQ ID NO: 14 - protein, 28 aa, Bacillus sp.
MFKFKKKFLVGLTAAFMSISMFSATASA

SEQ ID NO: 15 - protein, 21 aa, E. Coli
MKQSTIALALLPLLFTPVTKA

SEQ ID NO: 16 - protein, 22 aa, E. Coli
MMKRNILAVIVPALLVAGTANA

SEQ ID NO: 17 - protein, 21 aa, E. Coli
MKKSTLALVVMGIVASASVQA

SEQ ID NO: 18 - protein, 26 aa, E. Coli
MKIKTGARILALSALTTMMFSASALA

SEQ ID NO: 19 - protein, 21 aa, E. Coli
MKVKVLSLLVPALLVAGAANA

SEQ ID NO: 20 - protein, 20 aa, E. Coli
MKATKLVLGAVILGSTLLAG

SEQ ID NO: 21 - protein, 25 aa, E. Coli
MMITLRKLPLAVAVAAGVMSAQAMA

SEQ ID NO: 22 - protein, 20 aa, E. Coli
MRAKLLGIVLTTPIAISSFA

SEQ ID NO: 23 - protein, 25 aa, E. Coli
SKQHYGIRKYKVGVCSALIALSILG

SEQ ID NO: 24 - DNA, 1065 bp, Arabidopsis thaliana
gaccacgtgtacaagggtcagctgcaagcgtatgcgctgcagcacaacctggagctgccggtttacgcgaacgagcgtgaaggtccgccgcacgcgccgcgtttccgttgcaacgtgaccttctgcggccagacctttcaaagcagcgagttctttccgaccctgaaaagcgcggaacacgcggcggcgaagatcgcggtggcgagcctgaccccgcaaagcccggaaggtatcgatgttgcgtacaaaaacctgctgcaggagattgcgcaaaaggaaagcagcctgctgccgttctatgcgaccgcgaccagcggtccgagccacgcgccgacctttaccagcaccgtggagttcgcgggtaaagtttttagcggcgaggaagcgaagaccaagaaactggcggaaatgagcgcggcgaaagttgcgttcatgagcattaagaacggtaacagcaaccagaccggtagcccgaccctgccgagcgagcgtcaagaagacgtgaacagcaacgttaaaagcagcccgcaggagatccacagccaaccgagcagcaaggtggttatgaccccggacaccccgagcaaaggtattaaggttaacgaggatgaatttccggacctgcacgatgcgccggcgagcaacgcgaaagaaatcaacgtggcgctgaacgagccggaaaacccgaccaacgacggtaccctgagcgcgctgaccaccgatggcatgaagatgaacatcgcgagcagcagcctgccgattccgcacaacccgaccaacgttattaccctgaacgcgccggcggcgaacggtatcaagcgtaacattgcggcgtgcagcagctggatgccgcagaacccgaccaacgacggcagcgagaccagcagctgcgtggttgatgagagcgaaaagaaaaagctgatcatgggtaccggtcacctgagcattccgaccggtcagcacgtggtttgccgtccgtggaacccggagatcaccctgccgcaagatgcggaaatgctgttccgtgacgataaatttattgcgtatcgtctggtgaagccgtaa

SEQ ID NO: 25 - protein, 355 aa, Arabidopsis thaliana
MDHVYKGQLQAYALQHNLELPVYANEREGPPHAPRFRCNVTFCGQTFQSSEFFPTLKSAEHAAAKIAVASLTPQSPEGIDVAYKNLLQEIAQKESSLLPFYATATSGPSHAPTFTSTVEFAGKVFSGEEAKTKKLAEMSAAKVAFMSIKNGNSNQTGSPTLPSERQEDVNSNVKSSPQEIHSQPSSKVVMTPDTPSKGIKVNEDEFPDLHDAPASNAKEINVALNEPENPTNDGTLSALTTDGMKMNIASSSLPIPHNPTNVITLNAPAANGIKRNIAACSSWMPQNPTNDGSETSSCVVDESEKKKLIMGTGHLSIPTGQHVVCRPWNPEITLPQDAEMLFRDDKFIAYRLVKP

SEQ ID NO: 26 - DNA, 739 bp, Arabidopsis thaliana
aagcaagaaacactgcagcgagctgctgccgaacaagatgttccgtaaccaggacagcaagtacctgatcccggttcaaaaagaagcgccgccggtgaccaccctgccgatgaaggcgagcaccgttaaaagcccgcacaactgcgaggcgatcctgcgtgacgcggatccgccgattagcctgagcagcgttaacctgagcgaacagctgcgtagcggcgtgttcctgaagccgaagaaacaaatcaaatactgggttgatgagcgtaacagcaactgcttcatgctgtttgcgaagaacctgagcattacctggagcgacgatgtgaactattggacctggtttaccgagaaagaaagcccgaacgagaacgttgaagcggtgggtctgaagaacgtgtgctggctggacatcaccggcaaattcgatacccgtaacctgaccccgggtattgtttacgaggtggtttttaaggtgaaactggaagacccggcgtatggctgggataccccggttaacctgaaactggtgctgccgaacggcaaggagaaaccgcaggaaaagaaagttagcctgcgtgaactgccgcgttacaagtgggtggacgttcgtgtgggcgagttcgtgccggagaagagcgcggcgggcgagatcacctttagcatgtatgaacacgcggcgggtgtttggaagaaaggcctgagcctgaagggtgtggcgattcgtccgaaacaataa

SEQ ID NO: 27 - protein, 246 aa, Arabidopsis thaliana
MSKKHCSELLPNKMFRNQDSKYLIPVQKEAPPVTTLPMKASTVKSPHNCEAILRDADPPISLSSVNLSEQLRSGVFLKPKKQIKYWVDERNSNCFMLFAKNLSITWSDDVNYWTWFTEKESPNENVEAVGLKNVCWLDITGKFDTRNLTPGIVYEVVFKVKLEDPAYGWDTPVNLKLVLPNGKEKPQEKKVSLRELPRYKWVDVRVGEFVPEKSAAGEITFSMYEHAAGVWKKGLSLKGVAIRPKQ

SEQ ID NO: 28 - DNA, 14 bp, Artificial Sequence
  Kozak (Shine Delgarno) Sequence
aggaggtacccacc

SEQ ID NO: 29 - DNA, 2781 bp, Streptococcus thermophilus
atgaaacatattaatgattatttttgggctaagaaaacagaggaaaatagtagacttctttggttaccattaactcaacacttagaagacacgaaaaatattgcaggcctcttatgggaacattggttaagtgaaggacaaaaggtattaattgaaaattctattaatgttaaatcaaatattgaaaaccaagggaaaagattggcacaattcctaggagctgttcatgatatcggtaaagcaacaccagcttttcagacgcaaaaaggttatgcaaattcagtagatttggatattcaattgttagaaaaattggaacgcgcaggtttttctggcattagttctctccaactagcctcccccaaaaagagtcatcatagcattgcaggtcaatatttgttatcccattatggcgtggacgaagatattgcaacaattattggtggacaccatggacgaccagttgatgatttagacggtttaaattctcaaaaaagctatccctccaattattaccaggatgaaaagaaagatagtctcgtttatcagaaatggaagtcaaatcaagaagcttttttaaactgggctttaacagaaacagggtttaattctgtgtctcagcttccaaaaatcaaacagcctgctcaagttattctatcaggtttactcataatgtctgactggattgctagtaatgagcatttttttcctttgttaagtttggatgaaactgatgtgaaaaacaagagtcaacgtattgaaactgggtttaaaaagtggaaaaaatctaacttgtggcaacctgaaactttcgttgaccttgttactctttatcaggaaagatttggatttagtccacgaaattttcagctgatactctcacaaacaatcgaaaagacgactaatcctgggatagtgatactggaagcgccaatgggaatcgggaaaacagaggcggctctagcggtatcagagcagttatctagtaaaaaaggatgtagtggattgttttttggattgcccacacaagcaacctccaatggaatttttaagaggattgaacagtggacagagaatataaagggtaacaattctgatcatttttccattcagctggttcatggaaaagcagccttaaatacggattttattgagttacttaaaggaaatacaattaatatggacgactcggaaaacggcagtatttttgtcaatgagtggttttctgggagaaaaacttcagcattagatgattttgtagttgggacggtcgaccaatttttaatggtggctttaaaacaaaaacatttggccttacgtcatttaggatttagtaaaaaagttatcgttattgatgaagtccacgcttatgatgcttatatgagccaatatttgttggaagctatcagatggatgggagcttatggtgttcctgtaattattttatcagcaactttacctgcccaacaaagagaaaaactcataaaaagctatatggctggaatgggagtgaaatggcgagatattgaaaatatagatcagataaaaatagacgcataccctttaatcacttataatgacgggcctgacattcatcaagttaaaatgttcgaaaagcaagaacaaaaaaatatctacattcatcgtttaccagaagaacagttatttgatattgtaaaagaaggtcttgacaatggtggagtagttgggataattgtcaatacggtgagaaaatctcaagaattggcaagaaatttttcagatatttttggagatgatatggtagatttgcttcattctaatttcatagcaactgaaagaatccgaaaagaaaaggatttattgcaagaaattgggaaaaaagcaatacgtccaccaaagaaaatcattattggtacacaggtgcttgaacagtcgttagatattgattttgatgtactgataagcgacttagcgcctatggatttactcattcaacgtatcggacgactacatcgtcacaaaatcaaaaggccccaaaagcacgaagtagcaagattttatgttttaggaacatttgaagagtttgattttgatgaaggaacgcgtttggtttatggggactacctattagctagaactcagtactttttaccagataaaatacgacttcctgatgatatttcaccgctagtccaaaaggtttataattcagacctaacaattacgtttccaaagccagaacttcataaaaaatatttggatgctaaaatagaacatgatgataagattaaaaataaagaaacaaaggcaaagtcataccgtattgctaatcctgtcttaaaaaaatcgagagttcgaactaacagtttgattggttggttaaagaacctccatccaaatgatagtgaagaaaaagcatatgctcaagttcgagatattgaagatacagttgaagtgattgcattaaaaaaaatatctgatgggtatggtttgttcatagaaaataaagatatatctcagaacattactgatcctataattgcaaaaaaggtagcacaaaatactttacgacttccgatgagtttatccaaagcctataatattgatcaaacgattaatgagcttgaaagatataacaatagccacttaagtcaatggcaaaactcatcatggttaaagggatctcttgggattatttttgataaaaacaatgagtttatactgaatggatttaaactattatatgatgaaaaatatggtgttaccatagaaaggttggataagaatgagtcggtttaa

SEQ ID NO: 30 - protein, 926 aa, Streptococcus thermophilus
MKHINDYFWAKKTEENSRLLWLPLTQHLEDTKNIAGLLWEHWLSEGQKVLIENSINVKSNIENQGKRLAQFLGAVHDIGKATPAFQTQKGYANSVDLDIQLLEKLERAGFSGISSLQLASPKKSHHSIAGQYLLSHYGVDEDIATIIGGHHGRPVDDLDGLNSQKSYPSNYYQDEKKDSLVYQKWKSNQEAFLNWALTETGFNSVSQLPKIKQPAQVILSGLLIMSDWIASNEHFFPLLSLDETDVKNKSQRIETGFKKWKKSNLWQPETFVDLVTLYQERFGFSPRNFQLILSQTIEKTTNPGIVILEAPMGIGKTEAALAVSEQLSSKKGCSGLFFGLPTQATSNGIFKRIEQWTENIKGNNSDHFSIQLVHGKAALNTDFIELLKGNTINMDDSENGSIFVNEWFSGRKTSALDDFVVGTVDQFLMVALKQKHLALRHLGFSKKVIVIDEVHAYDAYMSQYLLEAIRWMGAYGVPVIILSATLPAQQREKLIKSYMAGMGVKWRDIENIDQIKIDAYPLITYNDGPDIHQVKMFEKQEQKNIYIHRLPEEQLFDIVKEGLDNGGVVGIIVNTVRKSQELARNFSDIFGDDMVDLLHSNFIATERIRKEKDLLQEIGKKAIRPPKKIIIGTQVLEQSLDIDFDVLISDLAPMDLLIQRIGRLHRHKIKRPQKHEVARFYVLGTFEEFDFDEGTRLVYGDYLLARTQYFLPDKIRLPDDISPLVQKVYNSDLTITFPKPELHKKYLDAKIEHDDKIKNKETKAKSYRIANPVLKKSRVRTNSLIGWLKNLHPNDSEEKAYAQVRDIEDTVEVIALKKISDGYGLFIENKDISQNITDPIIAKKVAQNTLRLPMSLSKAYNIDQTINELERYNNSHLSQWQNSSWLKGSLGIIFDKNNEFILNGFKLLYDEKYGVTIERLDKNESV

SEQ ID NO: 31 - DNA, 4107 bp, Streptococcus
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 32 - protein, 1368 aa, Streptococcus [sequence omitted]

SEQ ID NO: 33 - protein, 1053 aa, Staphylococcus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 34 - DNA, 148 bp, Tobacco mosaic virus
gaattcgtcgattcggttgcagcatttaaagcggttgacaactttaaaagaaggaaaaagaaggttgaagaaaagggtgtagtaagtaagtataagtacagaccggagaagtacgccggtcctgattcgtttaatttgaaagaagaaa

SEQ ID NO: 35 - DNA, 453 bp, Nicotiana tabacum IRES
ggcacgaggctcccattaatatttcttcttctgtgtaattccattattctgtagtagattcacgtccgagtttaaagaagagagaaaactgaaaaggcagaaaattccagagctttagatttagccaaagatagttatggtcgtgttgttcttggtgaagattggcaaagtaggagccaatggaagaaactaagatcataatcaatcgccccaaaaacaaccttgttcattctatggtttttctcttcggtttctatgtttgggattgggaattcctcactgtccttttgcttttcagttattgctccttctaattttccctagctaggatcttctcaattaatttcctttttcattttcaactaactcataattagcccaaatcttcaaaagagttttgtgtaagttgatagacgtttagagaaacagagaaatacaggggaaaaacaaggg

SEQ ID NO: 36 - DNA, 192 bp, turnip mosaic potyvirus IRES
gggaaagcttgcatgcctgcaggtcgactctagaaaaatataaaaactcaacacaacatacacaaaacgattaaagcaaacacaatctttcaaagcattcaaagcattcaagcaatcaaagattttcaaatcttttgtcgttatcaaagcaatcaccaacaggatccaggatccccgggtggtcagtccctt

SEQ ID NO: 37 - DNA, 143 bp, tobacco etch virus IRES
aaataacaaatctcaacacaacatatacaaaacaaacgaatctcaagcaatcaagcattctacttctattgcagcaatttaaatcatttcttttaaagcaaaagcaattttctgaaaattttcaccatttacgaacgatagca

SEQ ID NO: 38 - DNA, 160 bp, satellite tobacco necrosis virus CITE
aaaaaaaaaaaagtaaagacaggaaactttactgactaactcccagaggttcacaatgttagtgatggggcgctgaaagatgcgtagctacccttctggagccacttcctggtggtaagcagaaatccaagggtacggtggtacggtggaaagcagtccc

SEQ ID NO: 39 - DNA, 25 bp, Artificial Sequence
  NtHSF1_IRES_F primer
ggcacgaggctcccattaatatttc

SEQ ID NO: 40 - DNA, 19 bp, Artificial Sequence
  TuMV_IRES_F primer
gggaaagcttgcatgcctg

SEQ ID NO: 41 - DNA, 29 bp, Artificial Sequence
  TEV_IRES_F primer
gaaataacaaatctcaacacaacatatac

SEQ ID NO: 42 - DNA, 20 bp, Artificial Sequence
  rpoA-for primer
gcaccaaagaaggcgttcag

SEQ ID NO: 43 - DNA, 20 bp, Artificial Sequence
  rpoA-rev primer
ggtcaggtggcagatcacat

SEQ ID NO: 44 - DNA, 63 bp, E. Coli
atgaaaaagacggcgattgctatcgctgtggcgcttgctggattcgccactgtagcacaagca

(19) United States
(12) Patent Application Publication
(10) Pub. No.: US 2021/0292775 A1
(43) Pub. Date: Sep. 23, 2021

(54) System and Methods for Engineering Bacteria Fit for Eukaryotic mRNA Production, Export, and Translation in a Eukaryotic Host

(71) Applicant: Pebble Labs Inc., Los Alamos, NM (US)

(72) Inventors: Richard Sayre, Los Alamos, NM (US); Pedro Costa-Nunes, Albuquerque, NM (US); Guohua Yin, Los Alamos, NM (US)

(21) Appl. No.: 17/257,837

(22) PCT Filed: Jul. 5, 2019
(86) PCT No.: PCT/US2019/040747

§ 371 Date: Jan. 4, 2021

Related U.S. Application Data
(60) Provisional application No. 62/693,963, filed on Jul. 4, 2018.

(51) Int. Cl. C12N 15/74; C12N 15/11; C12N 9/22; C12N 1/20

(52) U.S. Cl. CPC C12N 15/74; C12N 15/111; C12N 2310/20; C12N 1/20; C12N 9/22

(57) ABSTRACT

The inventive technology includes novel systems, methods, and compositions for the generation of genetically engineered prokaryotic organisms configured to produce eukaryotic-like mRNA that may be introduced to, and translated in a eukaryotic host. Additional aspects may include novel eukaryotic-like nucleotide constructs and mRNA molecules, as well as methods and systems for the efficient delivery from prokaryotic cells to target eukaryotic hosts cells. Still further aspects of the invention include systems, methods and compositions for the the non-integrative transformation of eukaryotic cell.

This International PCT Application claims the benefit of and priority to U.S. Provisional Application No. 62/693,963, filed Jul. 4, 2018. The entire specification and figures of the above-referenced application are hereby incorporated, in their entirety by reference.

### SEQUENCING LISTING

The instant application contains a Sequence Listing which has been submitted electronically in ASCII format and is hereby incorporated by reference in its entirety.

### TECHNICAL FIELD

The inventive technology includes novel systems, methods, and compositions for the generation of genetically engineered prokaryotic organisms configured to produce eukaryotic-like mRNA that may be introduced to, and translated in a eukaryotic host. Additional embodiments may include novel eukaryotic-like nucleotide constructs and mRNA molecules, helper proteins that facilitate mRNA mobilization and translation, as well as methods and systems for the efficient transport and non-integrative transformation of eukaryotic cell.

### BACKGROUND

A major limiting factor in the development of genetically modified eukaryotic organisms has been the difficult and extended process required for making safe, effective, and importantly, stable transgenic expression systems. To develop even one transgenic eukaryotic organism may take up to ten years, and will require significant monetary investment. To overcome this problem, it has been proposed to use prokaryotic systems to produce and transfer eukaryotic-like mRNAs to modify eukaryotic hosts which require significantly less time to develop. However, as discussed below, the functional, genetic, and regulatory differences between prokaryotic and eukaryotic transcription and translation systems represent significant hurdles in incorporating the two systems to generate new transgenic organisms.

The genetic information of the cell is stored and transmitted in the nucleotide sequence of the deoxyribonucleic acid (DNA). Expression of this information requires transcription of DNA into messenger ribonucleic acid (mRNA) molecules that carry specific and precise information to the cytoplasmic sites of protein synthesis. In all species, transcription begins with the binding of the RNA polymerase complex (or holoenzyme) to a special DNA sequence at the beginning of the gene known as the promoter. Activation of the RNA polymerase complex enables transcription initiation, and this is followed by elongation of the transcript. In turn, transcript elongation leads to clearing of the promoter, and the transcription process can begin yet again. Transcription can thus be regulated at two levels: the promoter level (cis regulation) and the polymerase level (trans regulation). These elements differ among bacteria and eukaryotes.

In eukaryotic cells the mRNA are synthesized in the nucleus, often as larger precursor molecules called heterogeneous nuclear RNA (hnRNA). In prokaryotic organisms, mRNA is translated into protein as soon as it is transcribed. Unlike eukaryotic cells, prokaryotes such as bacteria do not have a distinct nucleus that separates DNA from ribosomes, so there is no barrier to immediate translation. Indeed, in high-magnification images of bacteria generated by electron microscopy, ribosomes can be seen translating messenger RNAs that are still being transcribed from DNA. This process simply would not work in eukaryotic cells, in part because eukaryotic RNAs contain introns and exons and must be edited before translation can begin. The eukaryotic nucleus therefore provides a distinct compartment within the cell, allowing transcription and splicing to proceed prior to the beginning of translation. Thus, in eukaryotes, while transcription occurs in the nucleus, translation occurs in the cytoplasm. In other words, eukaryotic transcription and translation are spatially and temporally isolated.

The mRNA in the cytoplasm has several other identifying structural characteristics. In eukaryotic cells, mRNA is usually a monocistronic message that encodes only one polypeptide. The 5′ end is capped with a specific structure involving 7-methylguanosine linked through a 5′-triphosphate bridge to the 5′ end of the messenger sequence. A 5′-non-translated region, which may be quite short or hundreds of nucleotides in length, separates the cap and the translational start site, which contains an AUG codon. The leader sequences of most vertebrate mRNAs are 20 to 100 nucleotides in length. Usually the translational start site is the first AUG sequence encountered as the message is read from the 5′ to the 3′ end. The informational sequences that encode a polypeptide are then contiguous with the initiation signal. The polypeptide-encoding sequences continue until a specific translational termination site is reached, which is followed by a 3′ untranslated sequence of about 100 nucleotides in length, before the mRNA terminates in a polyadenylated tail. In eukaryotes, the initiating amino acid is methionine rather than N-formylmethionine. However, as in prokaryotes, a special tRNA participates in initiation. This aminoacyl-tRNA is called Met-tRNAᵢ or Met-tRNA_{f} (the subscript “i” stands for initiation, and “f” indicates that it can be formylated in vitro).

Prokaryotic mRNA differs from eukaryotic mRNA in a number of other significant ways. The 5′ terminus is not capped, but retains a terminal triphosphate from initiation of its synthesis by an RNA polymerase. Most prokaryotic mRNA are polycistronic, encoding several polypeptides, and can include more than one initiation AUG sequence. In each case, a ribosome-positioning sequence is located about 10 nucleotides upstream of the AUG initiation signal. An untranslated sequence follows the last coding sequence, but there is no polyadenylated 3′ tail.

The translational start site of eukaryotic mRNA is also called a Kozak sequence. An exemplary Kozak sequence may have the form of: ((gcc)gccRccAUGG. The most highly conserved position in this motif is the purine (which is most often an A) three nucleotides upstream of the ATG codon, which indicates the start of translation.

As alluded to above, in most prokaryotes, the purine-rich ribosome binding site known as the Shine-Dalgarno (S-D) sequence assists with the binding and positioning of the 30S ribosome component relative to the start codon on the mRNA through interaction with a pyrimidine-rich region of the 16S ribosomal RNA. The S-D sequence is located on the mRNA downstream from the start of transcription and upstream from the start of translation, typically from 4-14 nucleotides upstream of the start codon, and more typically from 8-10 nucleotides upstream of the start codon. A S-D sequence may generally have a six-base consensus sequence of 5′-AGGAGG-3′.

While the initiating codon in eukaryotes is always AUG, prokaryotes do not use a specific purine-rich sequence on the 5′ side to distinguish initiator AUGs from internal ones. Instead, the AUG nearest the 5′ end of mRNA is usually selected as the start site. A 40S ribosome attaches to the cap at the 5′ end of eukaryotic mRNA and searches for an AUG codon by moving step-by-step in the 3′ direction. This scanning process in eukaryotic protein synthesis is powered by helicases that hydrolyze ATP. Pairing of the anticodon of Met-tRNAᵢ with the AUG codon of mRNA signals that the target has been found. In almost all cases, eukaryotic mRNA has only one start site and hence is the template for a single protein. In contrast, as noted above, prokaryotic mRNA can have multiple S-D sequences and, hence, start sites, and it can serve as a template for the synthesis of several proteins.

Eukaryotes utilize many more initiation factors than do prokaryotes, and their interplay is much more intricate. The prefix eIF denotes a eukaryotic initiation factor. For example, eIF-4E is a protein that binds directly to the 7-methylguanosine cap, whereas eIF-4A is a helicase. The difference in initiation mechanism between prokaryotes and eukaryotes is, in part, a consequence of the difference in RNA processing. The 5′ end of mRNA is readily available to ribosomes immediately after transcription in prokaryotes. In contrast, pre-mRNA must be processed and transported to the cytoplasm in eukaryotes before translation is initiated. Thus, there is ample opportunity for the formation of complex secondary structures that must be removed to expose signals in the mature mRNA. The 5′ cap provides an easily recognizable starting point.

As can be seen from the above, there exist numerous structural, genetic, and regulatory differences between the transcription and translation of eukaryotic and prokaryotic mRNAs. For example, prokaryotic mRNAs cannot be translated by eukaryotic 80S ribosomes. Based on these dissimilarities, the ability to generate transgenic organisms that may incorporate and/or stably express mRNAs across these two kingdoms represents a practical and commercial limitation in the field of genetically modified organisms. Indeed, the foregoing problems regarding the trans-kingdom expression of mRNA may represent a long-felt need for an effective—and economical—solution to the same. While implementing elements may have been available, actual attempts to meet this need may have been lacking to some degree. This may have been due to a failure of those having ordinary skill in the art to fully appreciate or understand the nature of the problems and challenges involved.

As a result of this lack of understanding, attempts to meet these long-felt needs may have failed to effectively solve one or more of the problems or challenges here identified. These attempts may even have led away from the technical directions taken by the present inventive technology and may even result in the achievements of the present inventive technology being considered to some degree an unexpected result of the approach taken by some in the field.

As will be discussed in more detail below, the current inventive technology overcomes the problems that limit the trans-kingdom production of eukaryotic-like mRNAs produced in prokaryotes which may further be introduced to, and translated in a host or recipient eukaryotic cell. Indeed, the invention's eukaryotic-like mRNAs and novel delivery system allow for the controlled transformation of a eukaryotic host without the need for stable genetic integration. This novel system allows for the rapid modification of the host's metabolism, increased nutritional value of crops, as well as the need to perform genome editing, bypassing the need for stable integration of foreign DNA and the application of a GMO label. In addition, the novel use of symbiotic, endosymbiotic, probiotic or endophytic bacteria may provide a vehicle for stable or continuous non-integrative transformation of a eukaryotic host cell through the continual delivery of target eukaryotic-like RNA molecules.

### SUMMARY OF THE INVENTION

Generally, the inventive technology relates to novel strategies for the trans-kingdom production and delivery of mRNAs expressed in a donor prokaryotic organism that may be translated in a recipient eukaryotic host. In one preferred embodiment, the invention may include engineering and/or expressing one or more heterologous “eukaryotic-like mRNAs” in a prokaryote donor organism which may be translated into a functional protein in a recipient eukaryotic host cell.

Another aspect of the inventive technology may include a transient eukaryotic metabolic engineering strategy mediated by the production and delivery of eukaryotic-like mRNA in bacteria living within a eukaryotic host. In one preferred embodiment, the inventive technology may include the generation of a transient eukaryotic metabolic engineering mediated by the production and delivery of eukaryotic mRNA in a donor bacteria living within the host. In one preferred embodiment, the eukaryotic-like mRNA may be in expressed in a prokaryote donor organism which may be translated into a functional protein in a recipient eukaryotic host cell, wherein such functional protein may cause the modulation of a metabolic pathway in the recipient eukaryotic host. Such metabolic pathway modulation may result in the expression of a phenotypic change in the recipient eukaryotic host cell or host more generally.

Another aspect of the current invention may include systems and methods for the production of one or more novel mRNAs in a donor prokaryotic organism, such as bacteria, that is further capable of being exported out of the donor bacteria, taken up, and translated by a eukaryotic host cell. Such systems, methods, and compositions may be applicable to in vivo, as well as in vitro systems as generally described herein. In additional aspect, such systems, methods, and compositions may be applicable to plant, as well as animal, and in particular mammalian systems as generally described herein.

Yet another aspect of the current invention may include novel isolated polynucleotides, ribonucleotides, expression cassettes, expression vectors, and genetic constructs. In one preferred embodiment, the invention may include novel mRNA compositions synthesized from a DNA template in a prokaryotic organism, such as bacteria, that is further capable of being taken up and translated by a eukaryotic host. Additional embodiments may include novel isolated sequences and compositions of polynucleotides, ribonucleotides, expression cassettes, expression vectors, and genetic constructs.

Additional aims of the inventive technology may include transformed, or genetically modified prokaryotic organisms, such as bacteria or even cyanobacteria, which may be genetically modified to express one or more novel eukaryotic-like mRNAs as generally described herein. Additional embodiments may include non-transformed eukaryotic organisms such a plants, insects, and animals that have been introduced to one or more genetically modified prokaryotic organisms that express one or more eukaryotic-like RNAs. For example, in one embodiment, the invention may include a novel host eukaryotic organism that translates eukaryotic-like mRNAs that have been synthesized in genetically modified bacteria.

Yet another aspect of the current invention may include novel eukaryotic-like mRNAs produced in a prokaryotic microorganism, such as bacteria, that may further be configured to be capable of being exported out of the bacteria, for example through outer membrane vesicle (OVM) structures and/or taken up and translated by a eukaryotic host. In another preferred embodiment, the invention may include both systems and methods of generating novel eukaryotic-like mRNAs in bacteria that may further be symbiotic and/or probiotic with a eukaryotic host.

One aspect of the inventive technology may include the generation of a transient and/or non-integrative non-GMO genome editing system. In this preferred embodiment, the inventive technology may include systems, methods, and compositions for a stable, transient transformation system using donor prokaryotic organism(s), such as symbiotic and/or probiotic bacteria, that may live in and colonize a recipient eukaryotic host. In this preferred embodiment, a donor prokaryotic organism(s) may be engineered to synthesize and deliver eukaryotic-like mRNAs, or mRNAs capable of being synthesized in a prokaryotic organism that may be delivered and/or translated in a eukaryotic host. A donor prokaryote may be engineered to synthesize and/or deliver eukaryotic-like mRNAs that are engineered to produce targeted proteins, such as meganucleases, Zinc finger nuclease, and/or TALENS that may exhibit gene or genome editing functions within a eukaryotic host. In this embodiment, the genetically engineered donor prokaryote may facilitate the production of mRNAs for a gene or genome editing protein that may effectuate genome editing directly on the host. In one embodiment, a donor prokaryote may be engineered to synthesize and/or deliver eukaryotic-like mRNAs for gene or genome editing proteins which are under the control of an inducible or other promotor.

Another aspect of the inventive technology may include the generation of a transient and/or non-integrative non-GMO CRISPR (clustered regularly interspaced short palindromic repeat) mediated genome editing system. In this preferred embodiment, the inventive technology may include systems, methods, and compositions for a stable CRISPR/Cas9 or 3-mediated long-term transient transformation system using donor prokaryotic organism(s), such as symbiotic and/or probiotic bacteria, that may live in and colonize a recipient eukaryotic host. In one preferred embodiment, a donor prokaryotic organism may be engineered to synthesize eukaryotic-like mRNAs that may be translated in a eukaryotic host. A donor prokaryote may be engineered to synthesize and/or deliver eukaryotic-like mRNAs for a CRISPR/Cas9 system, along with one or more guide RNA sequences to a eukaryotic host. In this embodiment, the genetically engineered donor prokaryote may facilitate the production of mRNAs for CRISPR/Cas9 system plus a guide RNA sequence that may effectuate genome editing directly on the host. In one embodiment, for example, a donor prokaryote may be engineered to synthesize and/or deliver eukaryotic-like mRNAs for CRISPR/Cas9 system which is under the control of an inducible or other promotor.

Another aspect of the inventive technology may include systems, methods and compositions for a stable transient non-integrative transformation system using donor prokaryotic organism(s), such as symbiotic and/or probiotic and/or endophytic bacteria, that may live in and colonize a recipient eukaryotic host. In this preferred embodiment, a donor prokaryotic organism(s) may be engineered to synthesize eukaryotic-like mRNAs that may be delivered and/or translated in a recipient eukaryotic host. A donor prokaryote may be engineered to synthesize and/or deliver eukaryotic-like mRNAs that are engineered to produce proteins that are configured to generate a phenotypic, biochemical, metabolic, or other directed modulations in a recipient eukaryotic organism. For example, in one preferred embodiment, a donor prokaryotic organism may be engineered to synthesize and deliver eukaryotic-like mRNAs, or mRNAs to a eukaryotic host that, when translated, may induce a new phenotype. In one embodiment, such a phenotypic change may include increases in one or more metabolic or other growth pathways. Additional phenotypic changes may include physical, and/or biochemical changes not previously present in the wild-type host. Additional phenotypic changes may include enhanced, or even new, metabolic processes, or even the production of a non-naturally occurring compounds or other molecules of interest. Examples of such compounds and molecules, of interest may include vaccine or other disease resistant molecules that may provide enhanced pathogen resistance in the eukaryotic host. Additional examples may include the production of one or more toxins or other compounds that may be lethal to a specific pathogen, insect, or other pest.

Another aspect of the invention may include systems, methods and compositions for introducing genetically engineered prokaryotic organisms to a eukaryotic host(s). In one preferred embodiment, a eukaryotic host may be contacted with a donor prokaryotic organism, such as a symbiotic or probiotic bacteria, engineered to synthesize and deliver eukaryotic-like mRNAs. This contact or application can be accomplished prior to, or during pathogen exposure or infection, or may be applied in response to a market condition, for example a demand for a certain product, compound, molecule or trait expressed in a eukaryotic host, such as a plant or other commercial crop.

Another aspect of the invention may include systems, methods and compositions for administering genetically engineered prokaryotic organisms to a recipient eukaryotic plant host(s). In one preferred embodiment, a eukaryotic plant host may be contacted with a donor prokaryotic organism, such as a symbiotic or probiotic bacteria, engineered to synthesize and deliver eukaryotic-like mRNAs. This contact or application can be accomplished through one or more methods, such as: feeding, soaking, spraying, injecting, aerosolized disbursement, environmental aerosolized disbursement, environmental aerosolized disbursement in water sources, lyophilized application, freeze-dried application, microencapsulated application, desiccated application, application in an aqueous carrier, application in a solution, brushing, dressing, dripping, and/or coating. This administration, or introduction step may be accomplished prior to, or during pathogen exposure or infection, or may be applied in response to a market condition, for example a demand for a certain product, compound, molecule, or trait expressed in a plant host.

Another aspect of the invention may include the generation of eukaryotic hosts, such as plants, that express traits commonly provided through direct genetic modification of the host's genome. As noted above, in one preferred embodiment, a donor prokaryotic organism, such as endophytic bacteria, may be engineered to synthesize and deliver eukaryotic-like mRNAs that impart the trait, and/or phenotype desired from a traditional GMO, without requiring genotypic changes such as integration of one or more transgenes into the host's genome.

Another aim of the invention may include methods, systems, and compositions for the treatment of a disease condition, preferably in a human or plant. In this embodiment, prokaryotic bacteria may be genetically engineered to produce one or more eukaryotic-like mRNAs that may further be introduced to a cell, tissue, or patient exhibiting a disease condition. Such eukaryotic-like mRNAs may be taken up by the eukaryotic host and translated into a target protein, the expression of which causes a reduction and/or cessation of the disease condition.

Another aspect may include systems, methods and compositions for the improved transport of Euk-mRNAs from donor prokaryotes to recipient eukaryotic cells. In one preferred embodiment, co-expression of RNA binding helper proteins increase trans-kingdom transport efficiency of Euk-mRNAs from donor prokaryotes to recipient eukaryotic cells. Heterologous helper genes/proteins may include one or more RNA binding helper proteins, such as DRB4 (dsRNA binding protein 2) and/or PP2-A1 (phloem protein 2-A1) that may act as chaperone proteins during trans-kingdom delivery from a donor prokaryote to a recipient eukaryote cell. Such RNA binding helper proteins may further include bacterial secretion peptides (e.g. OmpA, HylA) and cloned into the backbones encoding the various IRES/CITE constructs further facilitating trans-kingdom delivery of Euk-mRNAs.

Additional preferred embodiments of the inventive technology may include, but not be limited to the following:

1. A method of transient CRISPR genome-editing comprising the steps of:
generating a genetically modified donor prokaryote expressing a heterologous nucleotide sequence operably linked to a promoter encoding a heterologous Euk-mRNA wherein said heterologous Euk-mRNA is not translatable in said donor prokaryote and further includes:
   at least one untranslated region (UTR) forming a ribosomal regulatory control region configured to facilitate recruitment of eukaryotic ribosomes;
   a coding region encoding at least one CRISPR-associated endonuclease that is competent to be translated in a eukaryote;
   removal of prokaryote ribosomal binding sites;
   a Kozak consensus sequence;
   a poly-adenylated (poly-A) region configured to facilitate Poly-A binding proteins;
expressing said heterologous Euk-mRNA in said donor prokaryote;
co-expressing at least one guide RNA (gRNA) configured to hybridize with a target genome sequence in a recipient eukaryote;
transporting said heterologous Euk-mRNA and said gRNA from said donor prokaryote to said recipient eukaryote; and
translating said CRISPR-associated endonuclease from said heterologous Euk-mRNA in said recipient eukaryote generating a heterologous CRISPR-associated endonuclease which associates with said gRNA form a complex and bind to said target genome sequence and execute a genome editing function in said eukaryote.
   2. The method of embodiment 1 wherein said coding region encoding at least one CRISPR-associated endonuclease comprises a coding region encoding a Cas9 protein.
   3. The method of embodiment 1 wherein said coding region encoding at least one CRISPR-associated endonuclease comprises a coding region encoding a CRISPR-associated endonuclease selected from the group consisting of: the amino acid sequence according to SEQ ID. NO. 30; the amino acid sequence according to SEQ ID. NO. 32; the amino acid sequence according to SEQ ID. NO. 33, the nucleotide sequence according to SEQ ID. NO. 29; and the nucleotide sequence according to SEQ ID. NO. 31.
   4. The method of embodiment 2 wherein said Euk-mRNA further includes a stabilization region comprising at least two hybridizable sequences positioned at the 5′ and 3′ ends of said Euk-mRNA respectively, which are further configured to form a hairpin loop.
   5. The method of embodiment 4 wherein said donor prokaryote comprises a donor bacterium.
   6. The method of embodiment 5 wherein said donor bacterium is selected from the group consisting of: a symbiotic donor bacterium; an endosymbiont donor bacterium; a endophytic donor bacterium; a probiotic donor bacterium; an enteric bacterium; a RNaseIII deficient donor bacterium; an donor bacterium having endogenous hyper-vesiculation activity; an donor bacterium having endogenous hyper-vesiculation activity; an donor bacterium genetically engineered to have hyper-vesiculation activity; B. subtilis strain CCB422; E. coli strain HT27; E. coli strain HT115; E. coli strain JC8031; and Enterobacter cloacae strain Ae003.
   7. The method of embodiment 4 wherein said promoter comprises an inducible promoter.
   8. The method of embodiment 4 and further comprising a Euk-mRNA having a eukaryotic stop signal.
   9. The method of embodiment 4 wherein said untranslated region (UTR) forming a ribosomal regulatory control region comprises a untranslated region (UTR) forming a ribosomal regulatory control region selected from the group consisting of: a Internal Ribosome Entry Sites (IRES) sequence; and a positioned cap independent translation element” (CITE) sequence.
   10. The method of embodiment 9 wherein said IRES sequence comprises an IRES sequence selected from the group consisting of: a tobacco mosaic virus IRES (crTMV); a tobacco etch virus IRES (TEV); a turnip mosaic potyvirus IRES (TuMV); a Nicotiana tabacum heat shock protein IRES (NtHSF) and an artificial IRES sequence.
   11. The method of embodiment 9 wherein said IRES sequence comprises an IRES sequence selected from the group consisting of: the nucleotide sequences according to SEQ ID NOs. 34-37
   12. The method of embodiment 9 wherein said CITE sequence comprises a CITE sequence selected from the group consisting of: a satellite tobacco necrosis virus (SNTV) CITE; and an artificial CITE sequence.
   13. The method of embodiment 9 wherein said CITE sequence comprises the nucleotide sequences according to SEQ ID NO. 38.
   14. The method of embodiment 4 wherein said Euk-mRNA further comprises at least one additional endogenous 3′ UTR configured to recruit protein complexes that facilitate eukaryote ribosome interaction.
   15. The method of embodiment 4 wherein said step of transporting said heterologous Euk-mRNA and said gRNA from said donor prokaryote to a recipient eukaryote comprises the step of transporting said heterologous Euk-mRNA and said gRNA from said donor prokaryote to a recipient eukaryote through outer-membrane vesicles (OMVs).
   16. The method of embodiment 4 and further comprising the step of generating a genetically modified donor prokaryote co-expressing with said Euk-mRNA and said gRNA a heterologous nucleotide sequence operably linked to a promoter encoding at least one heterologous helper gene encoding at least one helper protein configured to increase transport efficiency of said Euk-mRNAs and said gRNA from donor prokaryotes to recipient eukaryotic cells.
   17. The method of embodiment 16 wherein said at least one helper protein configured to increase transport efficiency of said Euk-mRNAs from donor prokaryotes to recipient eukaryotic cells comprises at least one chimeric RNA binding helper protein coupled with a bacterial secretion signal.
   18. The method of embodiment 17 wherein said at least one chimeric RNA binding helper protein coupled with a bacterial secretion signal is selected from the group consisting of: dsRNA binding protein 2 (DRB4) coupled with a OmpA bacterial secretion signal; and phloem protein 2-A1 (PP2-A1) coupled with a OmpA bacterial secretion signal.
   19. The method of embodiment 18 wherein said at least one chimeric RNA binding helper protein coupled with a bacterial secretion signal is selected from the group consisting of: the amino acid sequence of SEQ ID NO. 11 coupled with the amino acid sequence of SEQ ID NO. 25; and the amino acid sequence of SEQ ID NO. 11 coupled with the amino acid sequence of SEQ ID NO. 27.
   20. The method of embodiment 17 wherein said at least one helper protein configured to increase transport efficiency of said Euk-mRNAs from donor prokaryotes to recipient eukaryotic cells is selected from the group consisting of: the amino acid sequence according to SEQ ID NO. 25; the amino acid sequence according to SEQ ID NO. 27; the nucleotide sequence according to SEQ ID NO. 24; and the nucleotide sequence according to SEQ ID NO. 26.
   21. The method of embodiment 17 and 18 wherein said bacterial secretion signal comprises a bacterial secretion signal selected from the group consisting of: PelB (pectate lyase B) from Erwinia carotovora; OmpA (outer-membrane protein A); StII (heat-stable enterotoxin 2); Endoxylanase from Bacillus sp.; PhoA (alkaline phosphatase); OmpF (outer-membrane protein F); PhoE (outer-membrane pore protein E); MalE (maltose-binding protein); OmpC (outer-membrane protein C); Lpp (murein lipoprotein); LamB (λ receptor protein); OmpT (protease VII); LTB (heat-labile enterotoxin subunit B); and HylA (a-Haemolysin).
   22. The method of embodiment 17 wherein said bacterial secretion signal comprises a bacterial secretion signal selected from the group consisting of: an amino acid sequence according to SEQ ID NOs. 11-23.
   23. The method of embodiment 4 wherein said a stabilization region comprising two hybridizable sequences positioned at the 5′ and 3′ ends of said Euk-mRNA respectively, which are further configured to form a Euk-mRNA hairpin loop comprises at least two hybridizable GC-rich sequences positioned at the 5′ and 3′ ends of said Euk-mRNA respectively, which are further configured to form a hairpin Euk-mRNA loop structure.
   24. The method of embodiment 4 wherein said Euk-mRNA loop structure is configured to stabilize the Euk-mRNA molecule, prevent degradation, enhance transport efficiency, and not interfere with eukaryote ribosome binding and translation in said recipient eukaryote.
   25. The method of embodiment 4 wherein said Euk-mRNA loop structure is configured to promote translation in said recipient eukaryote.
   26. A genetically modified bacterium expressing a heterologous nucleotide sequence operably linked to a promoter encoding a heterologous Euk-mRNA wherein said heterologous Euk-mRNA is not translatable in said donor prokaryote and further includes:
at least one untranslated region (UTR) forming a ribosomal regulatory control region configured to facilitate recruitment of eukaryotic ribosomes;
a coding region encoding at least one CRISPR-associated endonuclease that is competent to be translated in a eukaryote;
removal of prokaryote ribosomal binding sites;
a Kozak consensus sequence; and
a poly-adenylated (poly-A) region configured to facilitate Poly-A binding proteins;
co-expressing at least one guide RNA (gRNA) configured to hybridize with a target genome sequence in a recipient eukaryote.
   27. The bacterium of embodiment 26 wherein said coding region encoding at least one CRISPR-associated endonuclease comprises a coding region encoding a Cas9 protein.
   28. The bacterium of embodiment 26 wherein said coding region encoding at least one CRISPR-associated endonuclease comprises a coding region encoding a CRISPR-associated endonuclease selected from the group consisting of: the amino acid sequence according to SEQ ID. NO. 30; the amino acid sequence according to SEQ ID. NO. 32; the amino acid sequence according to SEQ ID. NO. 33, the nucleotide sequence according to SEQ ID. NO. 29; and the nucleotide sequence according to SEQ ID. NO. 31.
   29. The bacterium of embodiment 27 wherein said Euk-mRNA further includes a stabilization region comprising at least two hybridizable sequences positioned at the 5′ and 3′ ends of said Euk-mRNA respectively, which are further configured to form a hairpin loop.
   30. The bacterium of embodiment 29 wherein said donor prokaryote comprises a donor bacterium.
   31. The bacterium of embodiment 30 wherein said donor bacterium is selected from the group consisting of: a symbiotic donor bacterium; an endosymbiont donor bacterium; a endophytic donor bacterium; a probiotic donor bacterium; an enteric bacterium; a RNaseIII deficient donor bacterium; an donor bacterium having endogenous hyper-vesiculation activity; an donor bacterium having endogenous hyper-vesiculation activity; an donor bacterium genetically engineered to have hyper-vesiculation activity; B. subtilis strain CCB422; E. coli strain HT27; E. coli strain HT115; E. coli strain JC8031; and Enterobacter cloacae strain Ae003.
   32. The bacterium of embodiment 29 wherein said promoter comprises an inducible promoter.
   33. The bacterium of embodiment 29 and further comprising a Euk-mRNA having a eukaryotic stop signal.
   34. The bacterium of embodiment 29 wherein said untranslated region (UTR) forming a ribosomal regulatory control region comprises a untranslated region (UTR) forming a ribosomal regulatory control region selected from the group consisting of: a Internal Ribosome Entry Sites (IRES) sequence; and a positioned cap independent translation element” (CITE) sequence.
   35. The bacterium of embodiment 34 wherein said IRES sequence comprises an IRES sequence selected from the group consisting of: a tobacco mosaic virus IRES (crTMV); a tobacco etch virus IRES (TEV); a turnip mosaic potyvirus IRES (TuMV); a Nicotiana tabacum heat shock protein IRES (NtHSF) and an artificial IRES sequence.
   36. The bacterium of embodiment 34 wherein said IRES sequence comprises an IRES sequence selected from the group consisting of: the nucleotide sequences according to SEQ ID NOs. 34-37.
   37. The bacterium of embodiment 34 wherein said CITE sequence comprises a CITE sequence selected from the group consisting of: a satellite tobacco necrosis virus (SNTV) CITE; and an artificial CITE sequence.
   38. The bacterium of embodiment 34 wherein said CITE sequence comprises the nucleotide sequences according to SEQ ID NO. 38.
   39. The bacterium of embodiment 29 wherein said Euk-mRNA further comprises at least one additional endogenous 3′ UTR configured to recruit protein complexes that facilitate eukaryote ribosome interaction.
   40. The bacterium of embodiment 29 wherein said step of transporting said heterologous Euk-mRNA and said gRNA from said donor prokaryote to a recipient eukaryote comprises the step of transporting said heterologous Euk-mRNA and said gRNA from said donor prokaryote to a recipient eukaryote through outer-membrane vesicles (OMVs).
   41. The bacterium of embodiment 29 and further comprising the step of generating a genetically modified donor prokaryote co-expressing with said Euk-mRNA and said gRNA a heterologous nucleotide sequence operably linked to a promoter encoding at least one heterologous helper gene encoding at least one helper protein configured to increase transport efficiency of said Euk-mRNAs and said gRNA from donor prokaryotes to recipient eukaryotic cells.
   42. The bacterium of embodiment 41 wherein said at least one helper protein configured to increase transport efficiency of said Euk-mRNAs from donor prokaryotes to recipient eukaryotic cells comprises at least one chimeric RNA binding helper protein coupled with a bacterial secretion signal.
   43. The bacterium of embodiment 42 wherein said at least one chimeric RNA binding helper protein coupled with a bacterial secretion signal is selected from the group consisting of: dsRNA binding protein 2 (DRB4) coupled with a OmpA bacterial secretion signal; and phloem protein 2-A1 (PP2-A1) coupled with a OmpA bacterial secretion signal.
   44. The bacterium of embodiment 43 wherein said at least one chimeric RNA binding helper protein coupled with a bacterial secretion signal is selected from the group consisting of: the amino acid sequence of SEQ ID NO. 11 coupled with the amino acid sequence of SEQ ID NO. 25; and the amino acid sequence of SEQ ID NO. 11 coupled with the amino acid sequence of SEQ ID NO. 27.
   45. The bacterium of embodiment 42 wherein said at least one helper protein configured to increase transport efficiency of said Euk-mRNAs from donor prokaryotes to recipient eukaryotic cells is selected from the group consisting of: the amino acid sequence according to SEQ ID NO. 25; the amino acid sequence according to SEQ ID NO. 27; the nucleotide sequence according to SEQ ID NO. 24; and the nucleotide sequence according to SEQ ID NO. 26.
   46. The bacterium of embodiment 42 and 43 wherein said bacterial secretion signal comprises a bacterial secretion signal selected from the group consisting of: PelB (pectate lyase B) from Erwinia carotovora; OmpA (outer-membrane protein A); StII (heat-stable enterotoxin 2); Endoxylanase from Bacillus sp.; PhoA (alkaline phosphatase); OmpF (outer-membrane protein F); PhoE (outer-membrane pore protein E); MalE (maltose-binding protein); OmpC (outer-membrane protein C); Lpp (murein lipoprotein); LamB (λ receptor protein); OmpT (protease VII); LTB (heat-labile enterotoxin subunit B); and HylA (a-Haemolysin).
   47. The bacterium of embodiment 42 wherein said bacterial secretion signal comprises a bacterial secretion signal selected from the group consisting of: an amino acid sequence according to SEQ ID NOs. 11-23.
   48. The bacterium of embodiment CE110 wherein said a stabilization region comprising two hybridizable sequences positioned at the 5′ and 3′ ends of said Euk-mRNA respectively, which are further configured to form a Euk-mRNA hairpin loop comprises at least two hybridizable GC-rich sequences positioned at the 5′ and 3′ ends of said Euk-mRNA respectively, which are further configured to form a hairpin Euk-mRNA loop structure.
   49. The bacterium of embodiment 29 wherein said Euk-mRNA loop structure is configured to stabilize the Euk-mRNA molecule, prevent degradation, enhance transport efficiency, and not interfere with eukaryote ribosome binding and translation in said recipient eukaryote.
   50. The bacterium of embodiment 29 wherein said Euk-mRNA loop structure is configured to promote translation in said recipient eukaryote.
   51. A method of transient CRISPR genome-editing comprising the steps of:
generating a genetically modified donor prokaryote expressing a heterologous nucleotide sequence operably linked to a promoter encoding a heterologous Euk-mRNA wherein said heterologous Euk-mRNA is not translatable in said donor prokaryote and further includes:
   at least one untranslated region (UTR) forming a ribosomal regulatory control region configured to facilitate recruitment of eukaryotic ribosomes;
   a coding region encoding at least one gene-editing endonuclease that is competent to be translated in a eukaryote and configured to target a genome sequence;
   removal of prokaryote ribosomal binding sites;
   a Kozak consensus sequence;
   a poly-adenylated (poly-A) region configured to facilitate Poly-A binding proteins;
expressing said heterologous Euk-mRNA in said donor prokaryote;
transporting said heterologous Euk-mRNA and from said donor prokaryote to said recipient eukaryote; and
translating said gene-editing endonuclease from said heterologous Euk-mRNA in said recipient eukaryote generating a heterologous gene-editing endonuclease which binds to said target genome sequence and execute a genome editing function in said eukaryote.
   52. The method of embodiment 51 wherein said Euk-mRNA further includes a stabilization region comprising at least two hybridizable sequences positioned at the 5′ and 3′ ends of said Euk-mRNA respectively, which are further configured to form a hairpin loop.
   53. The method of embodiment 52 wherein said coding region encoding at least one CRISPR-associated endonuclease comprises a coding region encoding a Cas9 protein.
   54. The method of embodiment 52 wherein said coding region encoding at least one CRISPR-associated endonuclease comprises a coding region encoding a CRISPR-associated endonuclease selected from the group consisting of: the amino acid sequence according to SEQ ID. NO. 30; the amino acid sequence according to SEQ ID. NO. 32; the amino acid sequence according to SEQ ID. NO. 33, the nucleotide sequence according to SEQ ID. NO. 29; and the nucleotide sequence according to SEQ ID. NO. 31.
   55. The method of embodiment 53 and further comprising the step of co-expressing at least one guide RNA (gRNA) configured to hybridize with said target genome sequence in a recipient eukaryote.
   56. The method of embodiment 52 wherein said gene-editing endonuclease comprises a gene-editing endonuclease selected from the group consisting of: CRISPR-associated endonuclease, Cas9, Cas3, a TALAN-associated endonuclease; a meganuclease; and a zinc-finger associated endonuclease.
   57. The method of embodiment 52 wherein said donor prokaryote comprises a donor bacterium.
   58. The method of embodiment 57 wherein said donor bacterium is selected from the group consisting of: a symbiotic donor bacterium; an endosymbiont donor bacterium; a endophytic donor bacterium; a probiotic donor bacterium; an enteric bacterium; a RNaseIII deficient donor bacterium; an donor bacterium having endogenous hyper-vesiculation activity; an donor bacterium having endogenous hyper-vesiculation activity; an donor bacterium genetically engineered to have hyper-vesiculation activity; B. subtilis strain CCB422; E. coli strain HT27; E. coli strain HT115; E. coli strain JC8031; and Enterobacter cloacae strain Ae003.
   59. The method of embodiment 52 wherein said promoter comprises an inducible promoter.
   60. The method of embodiment 52 and further comprising a Euk-mRNA having a eukaryotic stop signal.
   61. The method of embodiment 52 wherein said untranslated region (UTR) forming a ribosomal regulatory control region comprises a untranslated region (UTR) forming a ribosomal regulatory control region selected from the group consisting of: a Internal Ribosome Entry Sites (IRES) sequence; and a positioned cap independent translation element” (CITE) sequence.
   62. The method of embodiment 61 wherein said IRES sequence comprises an IRES sequence selected from the group consisting of: a tobacco mosaic virus IRES (crTMV); a tobacco etch virus IRES (TEV); a turnip mosaic potyvirus IRES (TuMV); a Nicotiana tabacum heat shock protein IRES (NtHSF) and an artificial IRES sequence.
   63. The method of embodiment 61 wherein said IRES sequence comprises an IRES sequence selected from the group consisting of: the nucleotide sequences according to SEQ ID NOs. 34-37.
   64. The method of embodiment 61 wherein said CITE sequence comprises a CITE sequence selected from the group consisting of: a satellite tobacco necrosis virus (SNTV) CITE; and an artificial CITE sequence.
   65. The method of embodiment 61 wherein said CITE sequence comprises the nucleotide sequences according to SEQ ID NO. 38.
   66. The method of embodiment 52 wherein said Euk-mRNA further comprises at least one additional endogenous 3′ UTR configured to recruit protein complexes that facilitate eukaryote ribosome interaction.
   67. The method of embodiment 52 wherein said step of transporting said heterologous Euk-mRNA from said donor prokaryote to a recipient eukaryote comprises the step of transporting said heterologous Euk-mRNA from said donor prokaryote to a recipient eukaryote through outer-membrane vesicles (OMVs).
   68. The method of embodiment 52 and further comprising the step of generating a genetically modified donor prokaryote co-expressing with said Euk-mRNA a heterologous nucleotide sequence operably linked to a promoter encoding at least one heterologous helper gene encoding at least one helper protein configured to increase transport efficiency of said Euk-mRNAs from donor prokaryotes to recipient eukaryotic cells.
   69. The method of embodiment 68 wherein said at least one helper protein configured to increase transport efficiency of said Euk-mRNAs from donor prokaryotes to recipient eukaryotic cells comprises at least one chimeric RNA binding helper protein coupled with a bacterial secretion signal.
   70. The method of embodiment 69 wherein said at least one chimeric RNA binding helper protein coupled with a bacterial secretion signal is selected from the group consisting of: dsRNA binding protein 2 (DRB4) coupled with a OmpA bacterial secretion signal; and phloem protein 2-A1 (PP2-A1) coupled with a OmpA bacterial secretion signal.
   71. The method of embodiment 70 wherein said at least one chimeric RNA binding helper protein coupled with a bacterial secretion signal is selected from the group consisting of: the amino acid sequence of SEQ ID NO. 11 coupled with the amino acid sequence of SEQ ID NO. 25; and the amino acid sequence of SEQ ID NO. 11 coupled with the amino acid sequence of SEQ ID NO. 27.
   72. The method of embodiment 69 wherein said at least one helper protein configured to increase transport efficiency of said Euk-mRNAs from donor prokaryotes to recipient eukaryotic cells is selected from the group consisting of: the amino acid sequence according to SEQ ID NO. 25; the amino acid sequence according to SEQ ID NO. 27; the nucleotide sequence according to SEQ ID NO. 24; and the nucleotide sequence according to SEQ ID NO. 26.
   73. The method of embodiment 69 and 70 wherein said bacterial secretion signal comprises a bacterial secretion signal selected from the group consisting of: PelB (pectate lyase B) from Erwinia carotovora; OmpA (outer-membrane protein A); StII (heat-stable enterotoxin 2); Endoxylanase from Bacillus sp.; PhoA (alkaline phosphatase); OmpF (outer-membrane protein F); PhoE (outer-membrane pore protein E); MalE (maltose-binding protein); OmpC (outer-membrane protein C); Lpp (murein lipoprotein); LamB (λ receptor protein); OmpT (protease VII); LTB (heat-labile enterotoxin subunit B); and HylA (a-Haemolysin).
   74. The method of embodiment 69 wherein said bacterial secretion signal comprises a bacterial secretion signal selected from the group consisting of: an amino acid sequence according to SEQ ID NOs. 11-23.
   75. The method of embodiment 52 wherein said a stabilization region comprising two hybridizable sequences positioned at the 5′ and 3′ ends of said Euk-mRNA respectively, which are further configured to form a Euk-mRNA hairpin loop comprises at least two hybridizable GC-rich sequences positioned at the 5′ and 3′ ends of said Euk-mRNA respectively, which are further configured to form a hairpin Euk-mRNA loop structure.
   76. The method of embodiment 75 wherein said Euk-mRNA loop structure is configured to stabilize the Euk-mRNA molecule, prevent degradation, enhance transport efficiency, and not interfere with eukaryote ribosome binding and translation in said recipient eukaryote.
   77. The method of embodiment 76 wherein said Euk-mRNA loop structure is configured to promote translation in said recipient eukaryote.
   78. A genetically modified bacterium expressing a heterologous nucleotide sequence operably linked to a promoter encoding a heterologous Euk-mRNA wherein said heterologous Euk-mRNA is not translatable in said donor prokaryote and further includes:
at least one untranslated region (UTR) forming a ribosomal regulatory control region configured to facilitate recruitment of eukaryotic ribosomes;
a coding region encoding at least one gene-editing endonuclease that is competent to be translated in a eukaryote and configured to target a genome sequence;
removal of prokaryote ribosomal binding sites;
a Kozak consensus sequence;
a poly-adenylated (poly-A) region configured to facilitate Poly-A binding proteins;
   79. The bacterium of embodiment 78 wherein said Euk-mRNA further includes a stabilization region comprising at least two hybridizable sequences positioned at the 5′ and 3′ ends of said Euk-mRNA respectively, which are further configured to form a hairpin loop.
   80. The method of embodiment 78 wherein said coding region encoding at least one CRISPR-associated endonuclease comprises a coding region encoding a Cas9 protein.
   81. The bacterium of embodiment 78 wherein said coding region encoding at least one CRISPR-associated endonuclease comprises a coding region encoding a CRISPR-associated endonuclease selected from the group consisting of: the amino acid sequence according to SEQ ID. NO. 30; the amino acid sequence according to SEQ ID. NO. 32; the amino acid sequence according to SEQ ID. NO. 33, the nucleotide sequence according to SEQ ID. NO. 29; and the nucleotide sequence according to SEQ ID. NO. 31.
   82. The bacterium of embodiment 80 and further comprising the step of co-expressing at least one guide RNA (gRNA) configured to hybridize with said target genome sequence in a recipient eukaryote.
   83. The bacterium of embodiment 78 wherein said gene-editing endonuclease comprises a gene-editing endonuclease selected from the group consisting of: CRISPR-associated endonuclease, Cas9, Cas3, a TALAN-associated endonuclease; a meganuclease, and a zinc-finger associated endonuclease.
   84. The bacterium of embodiment 79 wherein said donor prokaryote comprises a donor bacterium.
   85. The bacterium of embodiment 84 wherein said donor bacterium is selected from the group consisting of: a symbiotic donor bacterium; an endosymbiont donor bacterium; a endophytic donor bacterium; a probiotic donor bacterium; an enteric bacterium; a RNaseIII deficient donor bacterium; an donor bacterium having endogenous hyper-vesiculation activity; an donor bacterium having endogenous hyper-vesiculation activity; an donor bacterium genetically engineered to have hyper-vesiculation activity; B. subtilis strain CCB422; E. coli strain HT27; E. coli strain HT115; E. coli strain JC8031; and Enterobacter cloacae strain Ae003.
   86. The bacterium of embodiment 79 wherein said promoter comprises an inducible promoter.
   87. The bacterium of embodiment 79 and further comprising a Euk-mRNA having a eukaryotic stop signal.
   88. The bacterium of embodiment 79 wherein said untranslated region (UTR) forming a ribosomal regulatory control region comprises a untranslated region (UTR) forming a ribosomal regulatory control region selected from the group consisting of: a Internal Ribosome Entry Sites (IRES) sequence; and a positioned cap independent translation element” (CITE) sequence.
   89. The bacterium of embodiment 88 wherein said IRES sequence comprises an IRES sequence selected from the group consisting of: a tobacco mosaic virus IRES (crTMV); a tobacco etch virus IRES (TEV); a turnip mosaic potyvirus IRES (TuMV); a Nicotiana tabacum heat shock protein IRES (NtHSF) and an artificial IRES sequence.
   90. The bacterium of embodiment 88 wherein said IRES sequence comprises an IRES sequence selected from the group consisting of: the nucleotide sequences according to SEQ ID NOs. 34-37.
   91. The bacterium of embodiment 88 wherein said CITE sequence comprises a CITE sequence selected from the group consisting of: a satellite tobacco necrosis virus (SNTV) CITE; and an artificial CITE sequence.
   92. The bacterium of embodiment 88 wherein said CITE sequence comprises the nucleotide sequences according to SEQ ID NO. 38.
   93. The bacterium of embodiment 79 wherein said Euk-mRNA further comprises at least one additional endogenous 3′ UTR configured to recruit protein complexes that facilitate eukaryote ribosome interaction.
   94. The bacterium of embodiment 79 wherein said step of transporting said heterologous Euk-mRNA from said donor prokaryote to a recipient eukaryote comprises the step of transporting said heterologous Euk-mRNA from said donor prokaryote to a recipient eukaryote through outer-membrane vesicles (OMVs).
   95. The bacterium of embodiment 79 and further comprising the step of generating a genetically modified donor prokaryote co-expressing with said Euk-mRNA a heterologous nucleotide sequence operably linked to a promoter encoding at least one heterologous helper gene encoding at least one helper protein configured to increase transport efficiency of said Euk-mRNAs from donor prokaryotes to recipient eukaryotic cells.
   96. The bacterium of embodiment 95 wherein said at least one helper protein configured to increase transport efficiency of said Euk-mRNAs from donor prokaryotes to recipient eukaryotic cells comprises at least one chimeric RNA binding helper protein coupled with a bacterial secretion signal.
   97. The method of embodiment 96 wherein said at least one chimeric RNA binding helper protein coupled with a bacterial secretion signal is selected from the group consisting of: dsRNA binding protein 2 (DRB4) coupled with a OmpA bacterial secretion signal; and phloem protein 2-A1 (PP2-A1) coupled with a OmpA bacterial secretion signal.
   98. The bacterium of embodiment 97 wherein said at least one chimeric RNA binding helper protein coupled with a bacterial secretion signal is selected from the group consisting of: the amino acid sequence of SEQ ID NO. 11 coupled with the amino acid sequence of SEQ ID NO. 25; and the amino acid sequence of SEQ ID NO. 11 coupled with the amino acid sequence of SEQ ID NO. 27.
   99. The bacterium of embodiment 98 wherein said at least one helper protein configured to increase transport efficiency of said Euk-mRNAs from donor prokaryotes to recipient eukaryotic cells is selected from the group consisting of: the amino acid sequence according to SEQ ID NO. 25; the amino acid sequence according to SEQ ID NO. 27; the nucleotide sequence according to SEQ ID NO. 24; and the nucleotide sequence according to SEQ ID NO. 26.
   100. The bacterium of embodiment 96 and 97 wherein said bacterial secretion signal comprises a bacterial secretion signal selected from the group consisting of: PelB (pectate lyase B) from Erwinia carotovora; OmpA (outer-membrane protein A); StII (heat-stable enterotoxin 2); Endoxylanase from Bacillus sp.; PhoA (alkaline phosphatase); OmpF (outer-membrane protein F); PhoE (outer-membrane pore protein E); MalE (maltose-binding protein); OmpC (outer-membrane protein C); Lpp (murein lipoprotein); LamB (λ receptor protein); OmpT (protease VII); LTB (heat-labile enterotoxin subunit B); and HylA (a-Haemolysin).
   101. The bacterium of embodiment 98 wherein said bacterial secretion signal comprises a bacterial secretion signal selected from the group consisting of: an amino acid sequence according to SEQ ID NOs. 11-23.
   102. The bacterium of embodiment 79 wherein said a stabilization region comprising two hybridizable sequences positioned at the 5′ and 3′ ends of said Euk-mRNA respectively, which are further configured to form a Euk-mRNA hairpin loop comprises at least two hybridizable GC-rich sequences positioned at the 5′ and 3′ ends of said Euk-mRNA respectively, which are further configured to form a hairpin Euk-mRNA loop structure.
   103. The bacterium of embodiment 102 wherein said Euk-mRNA loop structure is configured to stabilize the Euk-mRNA molecule, prevent degradation, enhance transport efficiency, and not interfere with eukaryote ribosome binding and translation in said recipient eukaryote.
   104. The bacterium of embodiment 103 wherein said Euk-mRNA loop structure is configured to promote translation in said recipient eukaryote.
   105. A method of transient, non-integrative transformation of a eukaryotic host expressing a eukaryotic-like messenger ribonucleic acid molecule (Euk-mRNA) in a prokaryote that is competent for translation in a recipient eukaryote comprising the steps of:
generating a genetically modified donor prokaryote expressing a heterologous nucleotide sequence operably linked to a promoter encoding a heterologous Euk-mRNA wherein said heterologous Euk-mRNA is not translatable in said donor prokaryote and further includes:
   at least one untranslated region (UTR) forming a ribosomal regulatory control region configured to facilitate recruitment of eukaryotic ribosomes;
   at least one protein coding region that is competent to be translated in said eukaryote;
   removal of prokaryote ribosomal binding sites;
   a Kozak consensus sequence; and
   a poly-adenylated (poly-A) region configured to facilitate Poly-A binding proteins;
expressing said heterologous Euk-mRNA in said donor prokaryote;
transporting said heterologous Euk-mRNA from said donor prokaryote to said recipient eukaryote; and
translating said at least one protein coding region of said heterologous Euk-mRNA in said recipient eukaryote generating a heterologous protein.
   106. The method of embodiment 105 wherein said donor prokaryote comprises a donor bacterium.
   107. The method of embodiment 106 wherein said donor bacterium is selected from the group consisting of: a symbiotic donor bacterium; an endosymbiont donor bacterium; a endophytic donor bacterium; a probiotic donor bacterium; an enteric bacterium; a RNaseIII deficient donor bacterium; an donor bacterium having endogenous hyper-vesiculation activity; an donor bacterium having endogenous hyper-vesiculation activity; an donor bacterium genetically engineered to have hyper-vesiculation activity; B. subtilis strain CCB422; E. coli strain HT27; E. coli strain HT115; E. coli strain JC8031; and Enterobacter cloacae strain Ae003.
   108. The method of embodiment 105 wherein said promoter comprises an inducible promoter.
   109. The method of embodiment 105 and further comprising a Euk-mRNA having a eukaryotic stop signal.
   110. The method of embodiment 105 wherein said untranslated region (UTR) forming a ribosomal regulatory control region comprises a untranslated region (UTR) forming a ribosomal regulatory control region selected from the group consisting of: a Internal Ribosome Entry Sites (IRES) sequence; and a positioned cap independent translation element” (CITE) sequence.
   111. The method of embodiment 110 wherein said IRES sequence comprises an IRES sequence selected from the group consisting of: a tobacco mosaic virus IRES (crTMV); a tobacco etch virus IRES (TEV); a turnip mosaic potyvirus IRES (TuMV); a Nicotiana tabacum heat shock protein IRES (NtHSF) and an artificial IRES sequence.
   112. The method of embodiment 105 wherein said IRES sequence comprises an IRES sequence selected from the group consisting of: the nucleotide sequences according to SEQ ID NOs. 34-37.
   113. The method of embodiment 105 wherein said CITE sequence comprises a CITE sequence selected from the group consisting of: a satellite tobacco necrosis virus (SNTV) CITE; and an artificial CITE sequence.
   114. The method of embodiment 105 wherein said CITE sequence comprises the nucleotide sequences according to SEQ ID NO. 38.
   115. The method of embodiment 105 wherein said Euk-mRNA further comprises at least one additional endogenous 3′ UTR configured to recruit protein complexes that facilitate eukaryote ribosome interaction.
   116. The method of embodiment 105 wherein said protein coding region comprises a protein coding region encoding a eukaryotic protein that further generates at least one of the following: a phenotypic change in said recipient eukaryote; a metabolic change in said recipient eukaryote; a biochemical change in said recipient eukaryote; increase growth; increase growth; enhances stress resistance; enhanced disease resistance; production of a non-naturally occurring compounds or other molecules; therapeutic pathogen bio-control; reduction in disease condition; a gene editing function.
   117. The method of embodiment 105 wherein said step of transporting said heterologous Euk-mRNA from said donor prokaryote to a recipient eukaryote comprises the step of transporting said heterologous Euk-mRNA from said donor prokaryote to a recipient eukaryote through outer-membrane vesicles (OMVs).
   118. The method of embodiment 105 wherein Euk-mRNA comprises a Euk-mRNA construct selected from the group consisting of: the nucleotide sequence according to SEQ ID NOs. 1-10, and wherein said protein coding region in said sequence is replaced with a target protein of interest.
   119. The method of embodiment 105 and further comprising the step of generating a genetically modified donor prokaryote co-expressing with said Euk-mRNA a heterologous nucleotide sequence operably linked to a promoter encoding at least one heterologous helper gene encoding at least one helper protein configured to increase transport efficiency of said Euk-mRNAs from donor prokaryotes to recipient eukaryotic cells.
   120. The method of embodiment 119 wherein said at least one helper protein configured to increase transport efficiency of said Euk-mRNAs from donor prokaryotes to recipient eukaryotic cells comprises at least one chimeric RNA binding helper protein coupled with a bacterial secretion signal.
   121. The method of embodiment 120 wherein said at least one chimeric RNA binding helper protein coupled with a bacterial secretion signal is selected from the group consisting of: dsRNA binding protein 2 (DRB4) coupled with a OmpA bacterial secretion signal; and phloem protein 2-A1 (PP2-A1) coupled with a OmpA bacterial secretion signal.
   122. The method of embodiment 121 wherein said at least one chimeric RNA binding helper protein coupled with a bacterial secretion signal is selected from the group consisting of: the amino acid sequence of SEQ ID NO. 11 coupled with the amino acid sequence of SEQ ID NO. 25; and the amino acid sequence of SEQ ID NO. 11 coupled with the amino acid sequence of SEQ ID NO. 27.
   123. The method of embodiment 119 wherein said at least one helper protein configured to increase transport efficiency of said Euk-mRNAs from donor prokaryotes to recipient eukaryotic cells is selected from the group consisting of: the amino acid sequence according to SEQ ID NO. 25; the amino acid sequence according to SEQ ID NO. 27; the nucleotide sequence according to SEQ ID NO. 24; and the nucleotide sequence according to SEQ ID NO. 26.
   124. The method of embodiment 120 and 121 wherein said bacterial secretion signal comprises a bacterial secretion signal selected from the group consisting of: PelB (pectate lyase B) from Erwinia carotovora; OmpA (outer-membrane protein A); StII (heat-stable enterotoxin 2); Endoxylanase from Bacillus sp.; PhoA (alkaline phosphatase); OmpF (outer-membrane protein F); PhoE (outer-membrane pore protein E); MalE (maltose-binding protein); OmpC (outer-membrane protein C); Lpp (murein lipoprotein); LamB (λ receptor protein); OmpT (protease VII); LTB (heat-labile enterotoxin subunit B); and HylA (a-Haemolysin).
   125. The method of embodiment 119 wherein said bacterial secretion signal comprises a bacterial secretion signal selected from the group consisting of: an amino acid sequence according to SEQ ID NOs. 11-23.
   126. The method of embodiment 105 wherein said Euk-mRNA further comprises a stabilization region comprising two hybridizable sequences positioned at the 5′ and 3′ ends of said Euk-mRNA respectively, which are further configured to form a Euk-mRNA hairpin loop.
   127. The method of embodiment 126 wherein said a stabilization region comprising two hybridizable sequences positioned at the 5′ and 3′ ends of said Euk-mRNA respectively, which are further configured to form a Euk-mRNA hairpin loop comprises at least two hybridizable GC-rich sequences positioned at the 5′ and 3′ ends of said Euk-mRNA respectively, which are further configured to form a hairpin Euk-mRNA loop structure.
   128. The method of embodiment 127 wherein said Euk-mRNA loop structure is configured to stabilize the Euk-mRNA molecule, prevent degradation, enhance transport efficiency, and not interfere with eukaryote ribosome binding and translation in said recipient eukaryote.
   129. The method of embodiment 127 wherein said Euk-mRNA loop structure is configured to promote translation in said recipient eukaryote.
   130. A method of non-integrative transformation of a eukaryotic host expressing an enhanced eukaryotic-like messenger ribonucleic acid molecule (Euk-mRNA) in a prokaryote that is competent for translation in a recipient eukaryote comprising the steps of:
generating a genetically modified donor prokaryote expressing a heterologous nucleotide sequence operably linked to a promoter encoding a heterologous Euk-mRNA wherein said heterologous Euk-mRNA is not translatable in said donor prokaryote and further includes:
   at least one untranslated region (UTR) forming a ribosomal regulatory control region configured to facilitate recruitment of eukaryotic ribosomes;
   at least one protein coding region that is competent to be translated in said recipient eukaryote;
   removal of prokaryote ribosomal binding sites;
   a Kozak consensus sequence;
   a stabilization region comprising at least two hybridizable sequences positioned at the 5′ and 3′ ends of said Euk-mRNA respectively, which are further configured to form a hairpin loop;
   a poly-adenylated (poly-A) region configured to facilitate Poly-A binding proteins;
expressing said heterologous Euk-mRNA in said donor prokaryote;
transporting said heterologous Euk-mRNA from said donor prokaryote to said recipient eukaryote; and
translating said at least one protein coding region of said heterologous Euk-mRNA in said recipient eukaryote generating a heterologous protein.
   131. The method of embodiment 130 wherein said donor prokaryote comprises a donor bacterium.
   132. The method of embodiment 131 wherein said donor bacterium is selected from the group consisting of: a symbiotic donor bacterium; an endosymbiont donor bacterium; a endophytic donor bacterium; a probiotic donor bacterium; an enteric bacterium; a RNaseIII deficient donor bacterium; an donor bacterium having endogenous hyper-vesiculation activity; an donor bacterium having endogenous hyper-vesiculation activity; an donor bacterium genetically engineered to have hyper-vesiculation activity; B. subtilis strain CCB422; E. coli strain HT27; E. coli strain HT115; E. coli strain JC8031; and Enterobacter cloacae strain Ae003.
   133. The method of embodiment 130 wherein said promoter comprises an inducible promoter.
   134. The method of embodiment 130 and further comprising a Euk-mRNA having a eukaryotic stop signal.
   135. The method of embodiment 130 wherein said untranslated region (UTR) forming a ribosomal regulatory control region comprises a untranslated region (UTR) forming a ribosomal regulatory control region selected from the group consisting of: a Internal Ribosome Entry Sites (IRES) sequence; and a positioned cap independent translation element” (CITE) sequence.
   136. The method of embodiment 135 wherein said IRES sequence comprises an IRES sequence selected from the group consisting of: a tobacco mosaic virus IRES (crTMV); a tobacco etch virus IRES (TEV); a turnip mosaic potyvirus IRES (TuMV); a Nicotiana tabacum heat shock protein IRES (NtHSF) and an artificial IRES sequence.
   137. The method of embodiment 135 wherein said IRES sequence comprises an IRES sequence selected from the group consisting of: the nucleotide sequences according to SEQ ID NOs. 34-37.
   138. The method of embodiment 135 wherein said CITE sequence comprises a CITE sequence selected from the group consisting of: a satellite tobacco necrosis virus (SNTV) CITE; and an artificial CITE sequence.
   139. The method of embodiment 135 wherein said CITE sequence comprises the nucleotide sequences according to SEQ ID NO. 38.
   140. The method of embodiment 130 wherein said Euk-mRNA further comprises at least one additional endogenous 3′ UTR configured to recruit protein complexes that facilitate eukaryote ribosome interaction.
   141. The method of embodiment 130 wherein said protein coding region comprises a protein coding region encoding a eukaryotic protein that further generates at least one of the following: a phenotypic change in said recipient eukaryote; a metabolic change in said recipient eukaryote; a biochemical change in said recipient eukaryote; increase growth; increase growth; enhances stress resistance; enhanced disease resistance; production of a non-naturally occurring compounds or other molecules; therapeutic pathogen bio-control; reduction in disease condition; a gene editing function.
   142. The method of embodiment 130 wherein said step of transporting said heterologous Euk-mRNA from said donor prokaryote to a recipient eukaryote comprises the step of transporting said heterologous Euk-mRNA from said donor prokaryote to a recipient eukaryote through outer-membrane vesicles (OMVs).
   143. The method of embodiment 130 wherein Euk-mRNA comprises a Euk-mRNA construct selected from the group consisting of: the nucleotide sequence according to SEQ ID NOs. 1-10, and wherein said protein coding region in said sequence is replaced with a target protein of interest.
   144. The method of embodiment 130 and further comprising the step of generating a genetically modified donor prokaryote co-expressing with said Euk-mRNA a heterologous nucleotide sequence operably linked to a promoter encoding at least one heterologous helper gene encoding at least one helper protein configured to increase transport efficiency of said Euk-mRNAs from donor prokaryotes to recipient eukaryotic cells.
   145. The method of embodiment B144240 wherein said at least one helper protein configured to increase transport efficiency of said Euk-mRNAs from donor prokaryotes to recipient eukaryotic cells comprises at least one chimeric RNA binding helper protein coupled with a bacterial secretion signal.
   146. The method of embodiment 145 wherein said at least one chimeric RNA binding helper protein coupled with a bacterial secretion signal is selected from the group consisting of: dsRNA binding protein 2 (DRB4) coupled with a OmpA bacterial secretion signal; and phloem protein 2-A1 (PP2-A1) coupled with a OmpA bacterial secretion signal.
   147. The method of embodiment 146 wherein said at least one chimeric RNA binding helper protein coupled with a bacterial secretion signal is selected from the group consisting of: the amino acid sequence of SEQ ID NO. 11 coupled with the amino acid sequence of SEQ ID NO. 25; and the amino acid sequence of SEQ ID NO. 11 coupled with the amino acid sequence of SEQ ID NO. 27.
   148. The method of embodiment 144 wherein said at least one helper protein configured to increase transport efficiency of said Euk-mRNAs from donor prokaryotes to recipient eukaryotic cells is selected from the group consisting of: the amino acid sequence according to SEQ ID NO. 25; the amino acid sequence according to SEQ ID NO. 27; the nucleotide sequence according to SEQ ID NO. 24; and the nucleotide sequence according to SEQ ID NO. 26.
   149. The method of embodiment 145 and 146 wherein said bacterial secretion signal comprises a bacterial secretion signal selected from the group consisting of: PelB (pectate lyase B) from Erwinia carotovora; OmpA (outer-membrane protein A); StII (heat-stable enterotoxin 2); Endoxylanase from Bacillus sp.; PhoA (alkaline phosphatase); OmpF (outer-membrane protein F); PhoE (outer-membrane pore protein E); MalE (maltose-binding protein); OmpC (outer-membrane protein C); Lpp (murein lipoprotein); LamB (λ receptor protein); OmpT (protease VII); LTB (heat-labile enterotoxin subunit B); and HylA (a-Haemolysin).
   150. The method of embodiment 130 wherein said bacterial secretion signal comprises a bacterial secretion signal selected from the group consisting of: an amino acid sequence according to SEQ ID NOs. 11-23.
   151. The method of embodiment 130 wherein said stabilization region comprising at least two hybridizable sequences positioned at the 5′ and 3′ ends of said Euk-mRNA respectively, which are further configured to form a hairpin loop comprises at least two hybridizable GC-rich sequences positioned at the 5′ and 3′ ends of said Euk-mRNA respectively, which are further configured to form a hairpin Euk-mRNA loop structure.
   152. The method of embodiment 151 wherein said Euk-mRNA loop structure is configured to stabilize said Euk-mRNA, prevent degradation of said Euk-mRNA, enhance transport efficiency of said Euk-mRNA, and not interfere with eukaryote ribosome binding and eukaryotic translation of said Euk-mRNA.
   153. The method of embodiment 151 wherein said Euk-mRNA loop structure is configured to promote translation in said recipient eukaryote.
   154. A eukaryotic-like messenger ribonucleic acid molecule (Euk-mRNA) configured to be expressed in in a donor prokaryote that is competent for translation in a recipient eukaryote wherein said Euk-mRNA comprises:
at least one untranslated region (UTR) forming a ribosomal regulatory control region configured to facilitate recruitment of eukaryotic ribosomes;
at least one protein coding region that is competent to be translated in a eukaryote;
a Kozak consensus sequence;
a poly-adenylated (poly-A) region; and
and wherein any prokaryote ribosomal binding sites have been removed.
   155. The Euk-mRNA of embodiment 154 wherein said at least one untranslated region (UTR) forming a ribosomal regulatory control region comprises a untranslated region (UTR) forming a ribosomal regulatory control region selected from the group consisting of: a Internal Ribosome Entry Sites (IRES) sequence; and a positioned cap independent translation element” (CITE) sequence.
   156. The Euk-mRNA of embodiment 155 wherein said IRES sequence comprises an IRES sequence selected from the group consisting of: a tobacco mosaic virus IRES (crTMV); a tobacco etch virus IRES (TEV); a turnip mosaic potyvirus IRES (TuMV); a Nicotiana tabacum heat shock protein IRES (NtHSF) and an artificial IRES sequence.
   157. The Euk-mRNA of embodiment 154 wherein said IRES sequence comprises an IRES sequence selected from the group consisting of: the nucleotide sequences according to SEQ ID NOs. 34-37.
   158. The Euk-mRNA of embodiment 154 wherein said CITE sequence comprises a CITE sequence selected from the group consisting of: a satellite tobacco necrosis virus (SNTV) CITE; and an artificial CITE sequence.
   159. The Euk-mRNA of embodiment 154 wherein said CITE sequence comprises the nucleotide sequences according to SEQ ID NO. 38.
   160. The Euk-mRNA of embodiment 154 wherein Euk-mRNA comprises a Euk-mRNA construct selected from the group consisting of: the nucleotide sequence according to SEQ ID NOs. 1-10, and wherein said protein coding region in said sequence is replaced with a target protein of interest.
   161. A method generating a genetically modified donor prokaryote co-expressing with said Euk-mRNA a heterologous nucleotide sequence operably linked to a promoter encoding at least one heterologous helper gene encoding at least one helper protein configured to increase transport efficiency of said Euk-mRNAs from donor prokaryotes to recipient eukaryotic cells.
   162. The method of embodiment 161 wherein said at least one helper protein configured to increase transport efficiency of said Euk-mRNAs from donor prokaryotes to recipient eukaryotic cells comprises at least one chimeric RNA binding helper protein coupled with a bacterial secretion signal.
   163. The method of embodiment 162 wherein said at least one chimeric RNA binding helper protein coupled with a bacterial secretion signal is selected from the group consisting of: dsRNA binding protein 2 (DRB4) coupled with a OmpA bacterial secretion signal; and phloem protein 2-A1 (PP2-A1) coupled with a OmpA bacterial secretion signal.
   164. The method of embodiment 163 wherein said at least one chimeric RNA binding helper protein coupled with a bacterial secretion signal is selected from the group consisting of: the amino acid sequence of SEQ ID NO. 11 coupled with the amino acid sequence of SEQ ID NO. 25; and the amino acid sequence of SEQ ID NO. 11 coupled with the amino acid sequence of SEQ ID NO. 27.
   165. The method of embodiment 161 wherein said at least one helper protein configured to increase transport efficiency of said Euk-mRNAs from donor prokaryotes to recipient eukaryotic cells is selected from the group consisting of: the amino acid sequence according to SEQ ID NO. 25; the amino acid sequence according to SEQ ID NO. 27; the nucleotide sequence according to SEQ ID NO. 24; and the nucleotide sequence according to SEQ ID NO. 26.
   166. The method of embodiments 162 and 163 wherein said bacterial secretion signal comprises a bacterial secretion signal selected from the group consisting of: PelB (pectate lyase B) from Erwinia carotovora; OmpA (outer-membrane protein A); StII (heat-stable enterotoxin 2); Endoxylanase from Bacillus sp.; PhoA (alkaline phosphatase); OmpF (outer-membrane protein F); PhoE (outer-membrane pore protein E); MalE (maltose-binding protein); OmpC (outer-membrane protein C); Lpp (murein lipoprotein); LamB (λ receptor protein); OmpT (protease VII); LTB (heat-labile enterotoxin subunit B); and HylA (a-Haemolysin).
   167. The method of embodiment 161 wherein said bacterial secretion signal comprises a bacterial secretion signal selected from the group consisting of: an amino acid sequence according to SEQ ID NOs. 11-23.
   lkj
   168. A eukaryotic-like messenger ribonucleic acid molecule (Euk-mRNA) configured to be expressed in in a donor prokaryote that is competent for translation in a recipient eukaryote wherein said Euk-mRNA comprises:
at least one untranslated region (UTR) forming a ribosomal regulatory control region configured to facilitate recruitment of eukaryotic ribosomes;
at least one protein coding region that is competent to be translated in a eukaryote;
a Kozak consensus sequence;
a poly-adenylated (poly-A) region;
a stabilization region comprising at least two hybridizable sequences positioned at the 5′ and 3′ ends of said Euk-mRNA respectively, which are further configured to form a Euk-mRNA hairpin loop structure; and
and wherein any prokaryote ribosomal binding sites have been removed.
   169. The Euk-mRNA of embodiment 168 wherein said stabilization region comprising at least two hybridizable sequences positioned at the 5′ and 3′ ends of said Euk-mRNA respectively, which are further configured to form a hairpin loop comprises at least two hybridizable GC-rich sequences positioned at the 5′ and 3′ ends of said Euk-mRNA respectively, which are further configured to form a hairpin Euk-mRNA loop structure.
   170. The Euk-mRNA of embodiment 169 wherein said Euk-mRNA loop structure is configured to stabilize said Euk-mRNA, prevent degradation of said Euk-mRNA, enhance transport efficiency of said Euk-mRNA, and not interfere with eukaryote ribosome binding and eukaryotic translation of said Euk-mRNA.
   171. The Euk-mRNA of embodiment 170 wherein said Euk-mRNA loop structure is configured to promote translation in said recipient eukaryote.
   172. A eukaryotic-like messenger ribonucleic acid molecule (Euk-mRNA) configured to be expressed in in a donor prokaryote that is competent for translation in a recipient eukaryote wherein said Euk-mRNA comprises:
eukaryotic regulatory and coding regions sufficient for translation of a target protein in a recipient prokaryote; and
a stabilization region comprising at least two hybridizable sequences positioned at the 5′ and 3′ ends of said Euk-mRNA respectively, which are further configured to form a Euk-mRNA hairpin loop structure.
   173. The Euk-mRNA of embodiment 172 wherein said stabilization region comprising at least two hybridizable sequences positioned at the 5′ and 3′ ends of said Euk-mRNA respectively, which are further configured to form a hairpin loop comprises at least two hybridizable GC-rich sequences positioned at the 5′ and 3′ ends of said Euk-mRNA respectively, which are further configured to form a hairpin Euk-mRNA loop structure.
   174. The Euk-mRNA of embodiment 173 wherein said Euk-mRNA loop structure is configured to stabilize said Euk-mRNA, prevent degradation of said Euk-mRNA, enhance transport efficiency of said Euk-mRNA, and not interfere with eukaryote ribosome binding and eukaryotic translation of said Euk-mRNA.
   175. The Euk-mRNA of embodiment 173 wherein said Euk-mRNA loop structure is configured to promote translation in said recipient eukaryote.
   176. A nucleotide sequence according to SEQ ID NOs. 1-10, and wherein said protein coding region in said sequence is replaced with a target protein of interest.
   177. A nucleotide sequence of embodiment 176 wherein said target protein of interest is a CRISPR-associated endonuclease.
   178. A nucleotide sequence of embodiment 177 wherein said CRISPR-associated endonuclease is Cas9 or Cas3.
   179. A nucleotide sequence of embodiment 178 and further comprising a nucleotide sequence encoding at lease one guide RNA (gRNA).
   180. An expression vector including the nucleotide sequence of embodiment 179.
   181. A bacteria transformed with the expression vector of embodiment 181.

### BRIEF DESCRIPTION OF DRAWINGS

The file of this patent contains at least one drawing/photograph executed in color. Copies of this patent with color drawing(s)/photograph(s) will be provided by the Office upon request and payment of the necessary fee.

FIG. 1A-E shows a schematic representation of bacterial nucleotide expression constructs that encode a linear eukaryotic-like RNA including the exemplary Internal Ribosome Entry Sites (IRES) and Cap Independent Translation Elements (CITE). (A-E) Schematic representation of Eukariotic RNA construct design used to validate competence of Internal Ribosome Entry site (IRES) sequences: crTMV, NtHSF, TuMV, TEV and Cap-Independent Translation Elements (CITE) sequence SNTV to recruit eukaryotic ribosomes and promote translation of a 2×GFP11 construct tagged with a signal peptide for cellular localization—in this case c-Myc tag for nuclear localization. Transcription in plant cells is under the control of a 35S CaMV promoter. A poly-A tail (50 nt) is included in this particular design following translation STOP in the case of IRES constructs and downstream of 3′UTR regulatory regions present in the SNTV CITE sequence. Transcription will result in formation of linear RNA molecule.

FIG. 2A-J shows a schematic representation of additional bacterial nucleotide expression constructs that encode a linear eukaryotic-like RNA including the exemplary Internal Ribosome Entry Sites (IRES) and Cap Independent Translation Elements (CITE), as well as nucleotide sequences encoding short CG-rich sequence upstream of 5′-UTR IRES and CITE UTR sequences. (A-J) Schematic representation of hairpin eukaryotic RNA constructs. Both bacterial expression and plant expression vectors were tested. Constructs for plant expression were driven by a 35S CaMV promoter whereas constructs for bacterial expression were driven by Ptac promoter. Important difference for the linear construct design presented in FIG. 1 is inclusion of a non-coding 5′-sequence tag that is homologous to a terminal 3′-UTR sequence (yellow boxes). Following transcription these two sequence tags will hybridize resulting in a hairpin like eukaryotic RNA. All other elements of the linear version of the constructs remained unchanged and included Internal Ribosome Entry site (IRES) sequences: crTMV, NtHSF, TuMV, TEV and Cap Independent Translation Elements (CITE) sequence SNTV. Eukaryotic translation of coding region will produce a 2×GFP11 construct tagged with a signal peptide for cellular localization. A poly-A tail (50 nt) is included in this particular design following translation STOP in the case of IRES constructs and downstream of 3′UTR regulatory regions present in the SNTV CITE sequence. As mentioned above, terminal part of the 3′ UTR will consist of the denominated 3′-paired-end (PE) terminal duplex.

FIG. 3 shows predicted RNA fold of two exemplary hairpin constructs. Represented are crTMV:NLS:2×GFP11 and TEV:NLS:2×GFP11. Highlighted are the paired-end termini duplex responsible for the hairpin conformation, Internal Ribosome entry sites (IRES)—RNA secondary structures that recruit eIF4G to the RNA followed by ribosome assembly, translation initiation site and poly-A stretch, RNA folds perform with RNAfold web server, with colors gradient representing pairing probability.

FIG. 4A-B (A) Identification of IRES:NLS:2×GFP11 coding Euk-mRNAs in host bacteria (E. coli HT115 and Enterobacter cloacae strain Ae003 Δrnc shown); and (B) presence of the coding sequences in isolated outer membrane vesicles (OMV) of HT115 bacteria. Identification of TuMV and NtHSF:NLS:2×GFP11 Euk-mRNAs in OMVs indicates secretion of RNAs for uptake by plant cells.

FIG. 5. Validation of 5′UTR Internal Ribosome Entry Site (IRES) and 3′ UTR Cap independent Translation Element (CITE) in driving GFP11 mRNA translation in eukaryotic cells. Construct were co-infiltrated in N. benthamiana leaves with control constructs encoding NLS:GFP1-10 (NLS nuclear localization signal). NLS-GFP11—control construct with plant ribosome binding sites; IRES—GFP11 constructs, with viral IRES sequences (TEV, crTMV, TuMV) and plant IRES (NtHSF), and viral 3′ CITE sequence (SNTV) drive translation of exemplary protein GFP11. Association of GFP11 to GFP 1-10 proteins reconstitutes full length GFP protein and fluorescence emission. Observation of GFP positive nuclei in all samples tested indicates construct design drives GFP11 translation in eukaryotic cells. Leaves were infiltrated with GV3101 and analyzed 2 dpi. ×20 magnification.

FIG. 6 Validation of TEV 5′ UTR Internal Ribosome Entry Site in driving GFP11 mRNA translation in eukaryotic cells. Both linear and hairpin version of TEV:NLS:2×GFP11 coding RNA were tested. Constructs were co-infiltrated in N. benthamiana leaves with control constructs encoding NLS:GFP1-10 (NLS—nuclear localization signal). UBQ:NLS:GFP11—control construct with plant ribosome binding sites driven by ubiquitin10 promoter. Association of GFP11 with GFP1-10 protein reconstitutes splitGFP and results in GFP-specific fluorescence emission. Observation of GFP positive nuclei in both TEV construct versions tested indicates construct design utilizing TEV for ribosomal recruitment successfully drives NLS:2×GFP11 translation in eukaryotic cells. All constructs were transformed into Agrobacterium tumefaciens GV3101 and infiltrated into N. benthamiana leaves and analyzed 2 dpi. ×20 magnification.

FIG. 7 Validation of hairpin Euk-mRNA design of 5′ UTR Internal Ribosome Entry Site (IRES) in driving GFP11 mRNA translation in plant cells. Constructs were co-infiltrated in N. benthamiana leaves with constructs encoding the complimentary half of GFP; NLS:GFP1-10 (NLS—nuclear localization signal). UBQ:NLS:GFP11—positive control construct with plant ribosome binding sites driven by ubiquitin10 promoter. hairpin IRES:NLS:2×GFP11 constructs (see FIG. 2), expression driven by 35S CaMV promoter, with viral IRES sequence TuMV and plant IRES NtHSF to drive translation of NLS:2×GFP11. Association of GFP11 with GFP1-10 protein reconstitutes split GFP protein (split-GFP) and results in fluorescence emission. Observation of GFP positive nuclei in all samples tested indicates construct hairpin design drives NLS:2×GFP11 translation in eukaryotic cells. No fluorescence is detected in leaves infiltrated with NtHSF:NLS:2×GFP11 in absence of GFP1-10 (negative control) All constructs were transformed into Agrobacterium tumefaciens GV3101 and infiltrated into N. benthamiana leaves and analyzed 2 dpi. ×20 magnification.

FIG. 8 (A) Identification of GFP11 and GFP1-10 peptides in N. benthamiana tissue infiltrated with GV3101 transformed with UBQ:NLS:GFP11:mCherry positive control); 35S:2×GFP11 construct with a plant 5′ UTR and a hairpin forming TEV:NLS:2×GFP11 construct (see also FIG. 6). Following GFP positive nuclei identification with a epifluorescence microscopy we performed further validation of NLS:2×GFP11 peptide presence in TEV:NLS:2×GFP11 leaf protein extracts. Unique peptides corresponding to GFP11 aa sequence were identified in all samples tested, which, importantly underlines competence of our hairpin IREs design in driving translation of RNAs in a eukaryotic cell. (B) Mass spectroscopy data showing standardized abundance of GFP11 in transient plant protein extracts.

FIG. 9 In planta validation of RNA trafficking from bacteria to plant cells and protein translation by the plant cell. Transgenic Arabidopsis thaliana plants expressing GFP1-10 were inoculated with E. coli HT115 and/or Enterobacter cloacae Ae003 mCherry and Δrnc strains expressing TEV:NLS:2×GFP11 hairpins. Identification of GFP positive nuclei (bright green spots) is indicative of reconstitution of split-GFP protein by interaction between the plant expressed GFP1-10 protein and the NLS:2×GFP11 peptide encoded in the hairpin TEV:NLS:2×GFP11 mRNA transcribed by bacteria and transferred to plant cells via outer membrane vesicle trafficking. Young A. thaliana GFP1-10 seedlings were co-incubated with bacteria for 1 hour, roots washed and seedlings plated in MS plates. Roots were visualized 2-3 days post inoculation. ×20 magnification.

FIG. 10A-B Confirmation of transcription of eukaryotic-mRNAs and bacterial translation of helper proteins in E. coli HT115 strain. These bacteria lines were subsequently used in the root inoculation to test effect of helper proteins on transfer of eukaryote-like coding RNA from bacteria to plants. A) Western blot readily identifies HA-tagged OmpA:DRB4 and OmpA:PP2-A1. B) RT-PCR demonstrating transcription of Euk-mRNAs of expected size—note that forward PCR primer used in each reaction was specific for the construct targeted as it anneals in the IRES sequence.

FIG. 11 Helper proteins improve delivery of eukaryotic-like RNA to plant cells. Arabidopsis thaliana roots were inoculated 3-5 days post-germination with E. coli HT115 strain transformed with TEV:NLS:2×GFP11 coding regions, with or without co-expression and bacterial translation of OmpA:3×HA:DRB4 and OmpA:3×HA:PP2-A1 RNA binding proteins. (A) representative root tips for each of the classes defined to classify efficiency of trans-kingdom transfer of Eukaryotic-like RNAs to plant cells. GFP positive nuclei were counted per each root analyzed and binned in defined classes. Data for all genotypes and constructs analyzed is compiled in table 1. (B) Representative root tips for each treatment are presented and illustrate increased number of GFP positive nuclei if eukaryotic-like RNA transfer to plant cells is chaperoned by RNA binding proteins DRB4 and PP2-A1. See also table 1 for quantitative data. Note difference of labeling between control root tips (Col-0) with roots from transgenic plants.

FIG. 12 shows schematic of transient Euk-mRNA CRISPR/Cas9-mediated transient gene-editing construct in one embodiment thereof.

FIG. 13 shows schematic representations of construct designs to incorporate bacterial expression of Cas3, Cas9 and TALENs enzymes for export and translation in target eukaryotic cells. Diagram represents the hairpin version but a linear RNA version is achievable by removing 5′ or 3′ Paired-end (PE) terminal duplex sequences. Cas3 and Cas9 constructs can also be associated with additional transcriptional units that encode non-coding guide RNAs to guide Cas proteins to desired eukaryotic host cell gDNA target region.

FIG. 14A-H shows schematic representations of construct designs that incorporate helper genes DRB4 and PP2-A1 coupled with an exemplary bacterial secretion signal which may be co-expressed with a Euk-mRNA in one embodiment thereof.

### DETAILED DESCRIPTION OF INVENTION

The following detailed description is provided to aid those skilled in the art in practicing the various embodiments of the present disclosure, including all the methods, uses, compositions, etc., described herein. Even so, the following detailed description should not be construed to unduly limit the present disclosure, as modifications and variations in the embodiments herein discussed may be made by those of ordinary skill in the art without departing from the spirit or scope of the present discoveries.

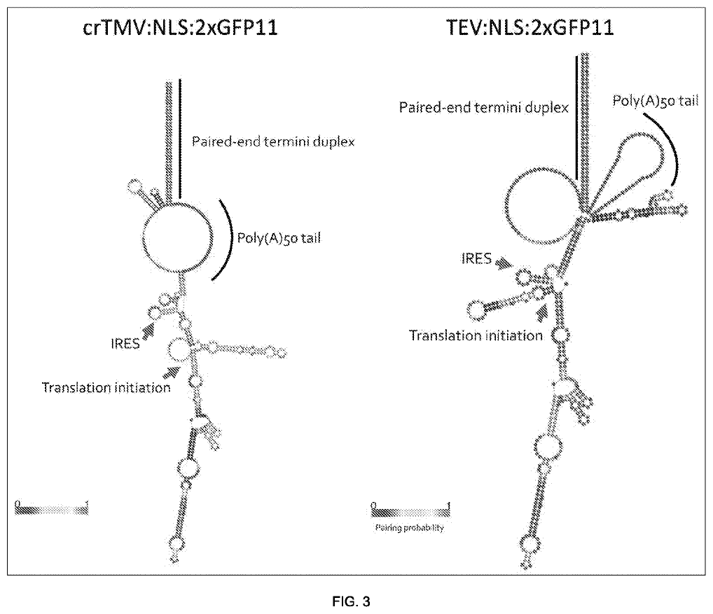

The inventive technology may include the generation of novel eukaryotic or eukaryotic-like mRNAs in an in vitro, or more preferably an in vivo system. In one embodiment, a eukaryotic-like mRNA may include any mRNA molecule that is capable of being synthesized by a prokaryotic organism and translated in a eukaryotic cell or system. On one preferred embodiment, the invention may include a generalized template for a eukaryotic-like mRNA that may be applicable to a number of gene targets. In this embodiment, a template for a eukaryotic-like mRNA may be synthesized from a template nucleic acid in a prokaryotic organism. In a preferred embodiment, a template eukaryotic-like mRNA may be expressed in a donor bacterium that may colonize a recipient eukaryotic host may include combination of regulatory (IRES, CITE, poly-A) and coding regions. In one preferred embodiment, a template eukaryotic-like mRNA may be expressed in a donor bacterium that may colonize a recipient eukaryotic host may include one or more of the following modifications: 1) removal of the bacterial Shine-Delgarno (S-D) or ribosome binding site (RBS) (generally located 8 bp upstream of the AUG initiation site) so as to impair loading of the mRNA on bacterial 70S ribosomes and its subsequent translation; 2a) addition of at least one a 5′ internal ribosomal binding site (IRES) which may allow for the recruitment of translation initiation factors, such as eIF proteins and assembly with the 80S ribosome in a recipient eukaryotic cell; 2b) alternative embodiments include the addition of “Cap Independent Translation Element” (CITE) sequences. CITEs are composed of 5′ and 3′ UTR structured RNA sequences that allow interaction between both ends of the coding RNA replicating the effect of interaction between Poly-A binding proteins (PABP) and translation initiation factors (eIF4G); 3) removal of prokaryote ribosomal binding sites; 4) addition of a poly-A tail at the end of the coding region to facilitate PABP binding and protect the RNA molecules against degradation; 5) a mRNA stabilization region, which in one preferred embodiment may include hybridizable regions at both the 5′ and 5′ regions that may form a hairpin loop RNA structure that stabilizes and prevents degradation of the eukaryotic-like mRNA, which allows for improved delivery from a prokaryote to a recipient eukaryotic cell. In one embodiment, this mRNA stabilization region may form a CG-rich sequence upstream of 5′-UTR IRES and CITE UTR sequences. This CG-rich sequence may pair with its anti-parallel sequence encoded at the terminus of the 3′ UTR of the construct's coding sequence forming a hairpin loop RNA, referred to sometime herein as a hairpin Euk-mRNA loop structure. This structure may stabilize the Euk-mRNA molecule, prevent degradation, and not interfere with ribosome binding and eukaryotic translation (See FIG. 3) and a coding region having a sequence for a target protein of interest.

Additional embodiments may include a eukaryotic-like RNA molecules having one or more of the following additional modifications: 1) addition of an 7G 5′CAP at the 5′ end of the eukaryotic mRNA. This CAP may allow for the recruitment of eIF proteins and assembly with the 80S ribosome in a recipient eukaryotic cell; 2) addition of polyadenylation recognition sequences in the 3′ UTR of the eukaryotic mRNA to stabilize the mRNA in eukaryotes; and 3) addition of a Kozak sequence to provide a translational start site in the recipient host.

In a preferred embodiment, the S-D sequence, or RBS, from the 5′ UTR of the targeted mRNA can be removed by synthesizing genes lacking this element. Similarly, one or more genes may be synthesized that include a Kozak sequence or other recognition sequences, so as to produce a eukaryotic-like mRNA that contains a translation start site to facilitate translation in a eukaryotic host.

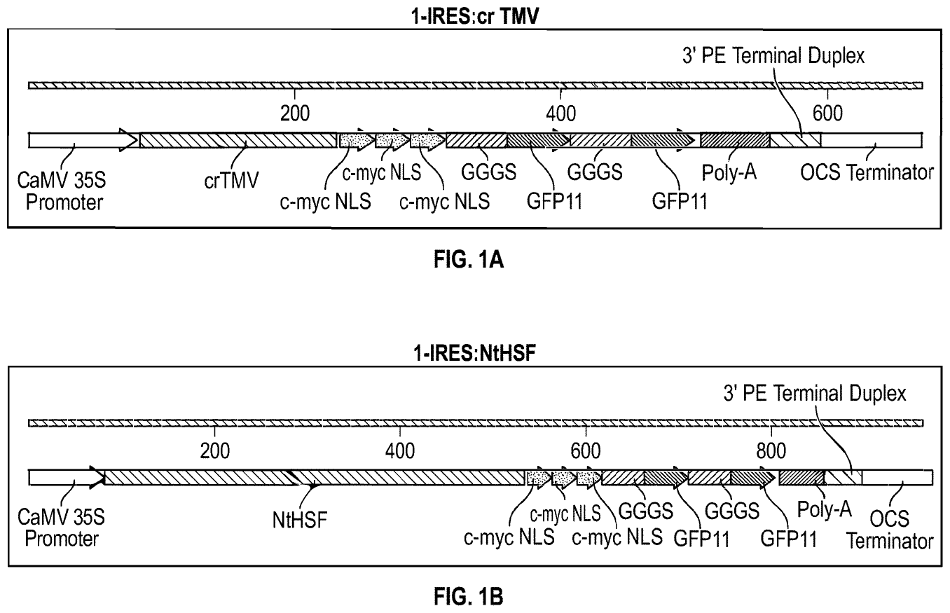

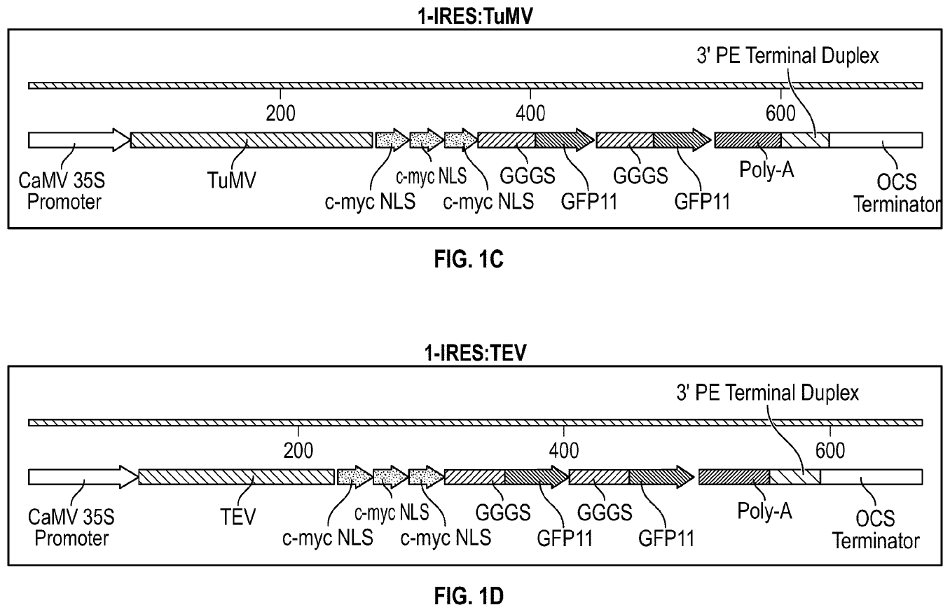

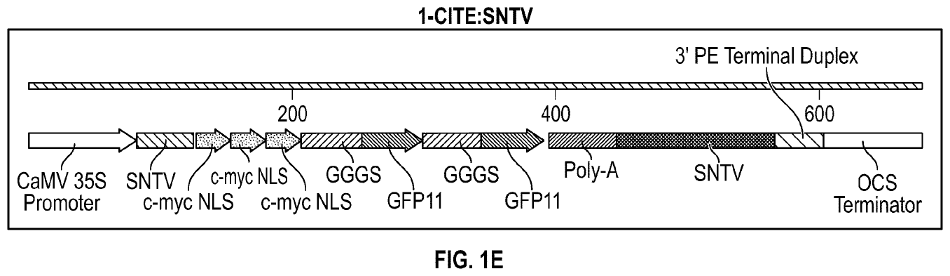

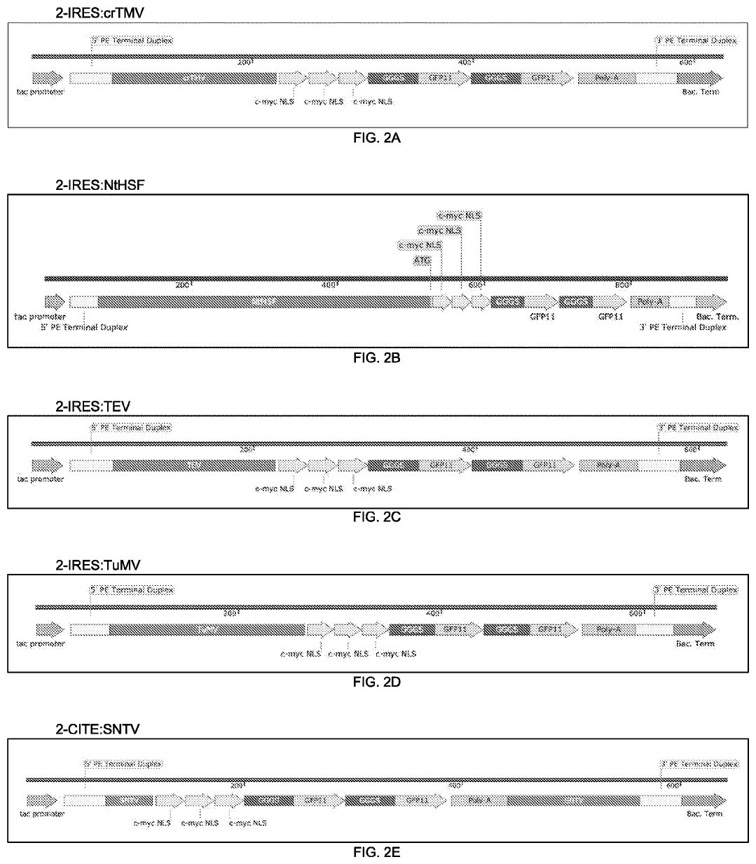

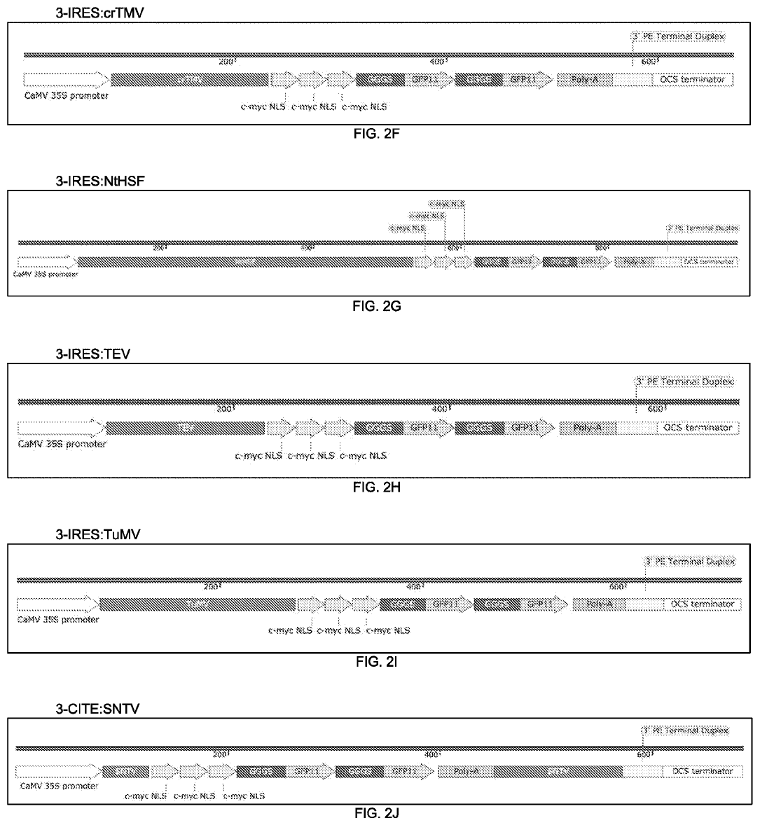

With respect to the addition of a 5′CAP, as generally shown in FIGS. 1-2, in one preferred embodiment a targeted eukaryotic-like mRNA may be engineered to incorporate a universal Internal Ribosome Entry Sites (IRES) in the 5′ UTR of the mRNA. In this configuration, the targeted mRNA may circumvent the generally understood requirement of a 5′-CAP to effectuate translation of mRNA in a eukaryotic host/system. Encoding an IRES sequence in the 5′UTR of the eukaryotic mRNA may further facilitate translation in the eukaryotic cell. A number of IRES sequences that may drive Cap-independent translation may include, but not be limited to: tobacco mosaic virus IRES (crTMV); tobacco etch virus IRES (TEV); turnip mosaic potyvirus IRES (TuMV); Nicotiana tabacum heat shock protein IRES (NtHSF); and satellite tobacco necrosis virus (SNTV) CITE; and an artificial IRES or CITE sequence.

The invention may also include methods, systems, and compositions to screen for appropriate functional regulator sequences that may be expressed in a eukaryotic-like mRNA. In this embodiment, one or more of the regulatory sequences identified herein may be incorporated into a Green Fluorescent Protein (GFP) exon encoding mRNA which may further include a polyadenylation recognition signal. In this configuration, the invention may act as a rapid screening system for the delivery of a eukaryotic-like mRNA from a donor prokaryote, such as a bacterium, that is translatable in the host organism. For example, in this system fluorescent donor-bacteria can indicate that translation of the eukaryotic-like mRNA is also occurring in the bacteria. As outlined below, identification of fluorescence in the receptor cell, and not in the bacteria, may indicate that the eukaryotic-like mRNA has been delivered to the host and that it is capable of serving as a template for eukaryotic ribosomes. In this system, GFP can be further tagged with localization signals (e.g. SV40 or Myc-tag for nuclear localization) to facilitate screening and validation of eukaryotic translation via microscopy, cell sorting, and the like.

Another embodiment of the invention may include systems, methods, and compositions for the stable mobilization of the engineered eukaryotic-like mRNA from a donor prokaryote, in this instance a bacteria, to the eukaryotic host, such as a plant or animal cell. This stable mobilization may include vesicular trafficking mechanisms within the donor. In this preferred embodiment, the eukaryotic-like mRNA may include paired termini translation competent constructs (ptRNA), stabilized by pairing of 5′ and 3′-end regions and including IRES sequences for ribosome recruitment and poly-adenylation recognition signals. In this embodiment, the IRES sequence(s) may facilitate recruitment of ribosomes to an engineered gene construct and allow translation in the recipient eukaryotic host. As described above, use of GFP coding sequences allows rapid screening and/or validation of various eukaryotic-like mRNA construct designs by fluorescence detection; successful export of ptGFP RNA from donor bacteria, uptake by the recipient cell, followed by ribosome recruitment and translation resulting in fluorescent recipient cells.

The invention may include exemplary linear Euk-mRNA over-expression constructs. FIGS. 1A-E show schematic representations of Eukaryotic mRNA construct design used to validate competence of various Internal Ribosome Entry site (IRES) sequences: crTMV, NtHSF, TuMV, TEV and the Cap-Independent Translation Elements (CITE) sequence SNTV to recruit eukaryotic ribosomes and promote translation of a 2×GFP11 construct tagged with a signal peptide (N-tyerminal c-Myc tag) for nuclear localization. Transient transcription in plant cells may be under the control of a 35S CaMV promoter. A poly-A tail (50 nt) may further included in this particular design following the translation STOP in the case of IRES constructs and downstream of 3′UTR regulatory regions present in the SNTV CITE sequence. Notably, as described above, transcription of such nucleotide sequences directed to such exemplary constructs will result in formation of linear RNA molecule.

The invention may include exemplary hairpin Euk-mRNA over-expression constructs. FIGS. 2A-J shows schematic representation of exemplary hairpin eukaryotic mRNA-GFP11 constructs. Both bacterial expression and plant expression vectors were used. Constructs for plant expression were driven by a 35S CaMV promoter whereas constructs for bacterial expression were driven by Ptac promoter. Important difference for the linear construct design presented in FIG. 1 is inclusion of a non-coding 5′-sequence tag that is homologous to a terminal 3′-UTR sequence (yellow boxes). Following transcription these two sequence tags will hybridize resulting in a hairpin like eukaryotic mRNA. All other elements of the linear version of the constructs are also present and include Internal Ribosome Entry site (IRES) sequences: crTMV, NtHSF, TuMV, TEV and Cap Independent Translation Elements (CITE) sequence SNTV. Eukaryotic translation of coding region may produce a 2×GFP11 construct tagged with a signal peptide for nuclear localization (N-tyerminal c-Myc tag). A poly-A tail (50 nt) is further included in these exemplary constructed following the translation STOP in the case of IRES constructs and downstream of 3′UTR regulatory regions present in the SNTV CITE sequence. Notably, as described above, transcription of such nucleotide sequences directed to such exemplary constructs will result in formation of linear RNA molecule. More specifically, in this embodiment, such CG-rich sequence may pair with its anti-parallel sequence encoded at the terminus of the 3′ UTR of the construct's coding sequence forming a hairpin loop RNA. This structure may stabilize the RNA molecule without interfering with ribosome binding and eukaryotic translation (See FIG. 3).

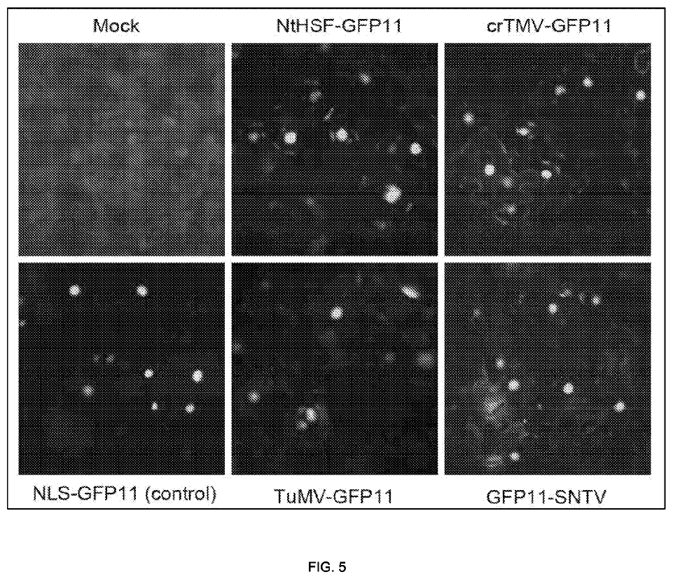

As shown generally in FIG. 5, validation of 5′ UTR Internal Ribosome Entry Site (IRES) and 3′ UTR Cap Independent Translation element (CITE) in driving GFP11 mRNA translation in eukaryotic cells. Constructs were co-infiltrated in N. benthamiana leaves. One half of the split GFP was encoded by NLS:GFP1-10 (NLS—nuclear localization signal). The UBQ:NLS:GFP11—construct encodes the complementary portion (GFP11) of the split GFP with plant ribosome binding sites driven by ubiquitin10 promoter. Other constructs include: 1) Linear IRES:NLS:2×GFP11 construct (see FIG. 1) whose expression was driven by 35S CaMV promoter, and containing viral IRES sequences (crTMV, TuMV), and 2) Plant IRES (NtHSF) and viral 3′ CITE sequence (SNTV) drive translation of NLS:2×GFP11. Association of GFP11 with GFP1-10 protein reconstitutes functional GFP protein (splitGFP) fluorescence emission. Observation of GFP positive nuclei in all samples tested indicates construct design drives NLS:2×GFP11 translation in eukaryotic cells as generally shown in the imaged figures. All constructs were transformed into Agrobacterium tumefaciens GV3101 and infiltrated into N. benthamiana leaves.

The invention may further include systems, methods and compositions for the efficient delivery of eukaryotic-like mRNAs from a donor prokaryotic organism to the recipient eukaryotic organism. In one preferred embodiment, the invention may include the expression of RNA binding proteins in bacteria tagged with secretion peptides—e.g. N-terminal OmpA or C-terminal HylA—which may aid in the secretion of mRNAs via membrane vesicles and ultimately facilitate mRNA uptake by the recipient eukaryotic cells. Naturally, additional secretion signals may be used as well. Preferably, the signal sequence is STII, OmpA, PhoE, LamB, MBP, PhoA or HylA among others. In this preferred embodiment, RNA binding by OmpA- or HylA tagged helper proteins may occur within the donor bacterial cell forming a ribonucleoprotein complex (RNP) competent for bacterial secretion. Additional embodiments may further be generically engineered into a donor bacterial organism to aid in the assembly of RNPs complexes within the bacterial cell. For example, in one embodiment this may include the co-expression of eIF4G fused to an RNA tethering domain and insertion of a RNA recognition sequence in the mRNA construct instead of IRES motifs. In this embodiment, once the RNP is formed between engineered eIF4G and coding mRNA, the RNP complex moves into the eukaryotic cell where ribosomes are recruited via interaction with eIF4G. This embodiment may allow for the selective export from a donor bacterium of only the eukaryotic-like mRNA of interest, and the direct recruitment of ribosomes upon uptake by the eukaryotic cell. Additional secretion signals may include a Tat secretion signal.

In an alternative embodiment, and in the case of ptRNA constructs, RNPs can be formed between OmpA tagged dsRNA binding protein (e.g. DRB4) and dsRNA region of the synthetic ptRNA. Export of RNP to eukaryotic cell, such as a plant cell and ribosome binding to IRES can result in translation of the target encoded protein. Construct design is not restricted to the embodiment described herein and can be modified to combine distinct RNA tethering sequences and IRES motifs, allowing different approaches to RNP assembly and CAP independent translation initiation in eukaryotic systems.

In another embodiment, co-expression of heterologous RNA binding helper proteins increases trans-kingdom transport efficiency of Euk-mRNAs from prokaryote to eukaryotic cells. As detailed below, one or more heterologous helper-proteins may be co-expressed in a donor prokaryote to facilitate trans-kingdom transport of the Euk-mRNAs to a recipient eukaryotic cell. Such heterologous helper genes nucleotide sequences may be operably linked with a promoter, and preferably an inducible promoter and may be co-expressed in a donor prokaryote, such as bacteria, with one or more Euk-mRNAs. In one embodiment, a heterologous helper gene may include a heterologous helper gene may encode a RNA binding helper proteins from A. thaliana, DRB4 (dsRNA binding protein 2) and/or PP2-A1 (phloem protein 2-A1) that may act as chaperone proteins during trans-kingdom delivery from a donor prokaryote to a recipient eukaryote cell. Such RNA binding helper proteins may further include bacterial secretion peptides (e.g. OmpA, HylA) and cloned into the backbones encoding the various IRES/CITE constructs as generally shown in FIGS. 1-2. Transcription of such helper genes/proteins may be driven from an independently operable promoter.

The inventive technology may include the generation of a transient gene editing system. In one preferred embodiment the invention may include a transient CRISPR/CAS9 mediated genome editing system. In this preferred embodiment, the inventive technology may include systems, methods and compositions for a stable CRISPR/Cas9-mediated transient transformation system using donor prokaryotic organism(s), such as symbiotic, endophytic, and/or probiotic bacteria, that may live in, and colonize a recipient eukaryotic host. In this preferred embodiment, a donor prokaryotic organism(s) may be engineered to synthesize eukaryotic-like mRNAs that may be translated in a eukaryotic host as generally described herein. Here, a donor prokaryote may be engineered to synthesize and/or introduce eukaryotic-like mRNAs encoding a CRISPR/Cas9 enzyme as well as a guide RNA (gRNA) sequence to a eukaryotic host. In this embodiment, the genetically engineered donor prokaryote may facilitate the production of eukaryotic-like mRNAs for a CRISPR/Cas9 system plus a gRNA sequence that may effectuate genome editing directly on the host. On one embodiment, for example, a donor prokaryote may be engineered to synthesize and/or deliver eukaryotic-like mRNAs, such as a Cas9 protein and a targeted gRNA, for CRISPR/Cas9-mediated gene editing which may further be under the control of an inducible promotor.

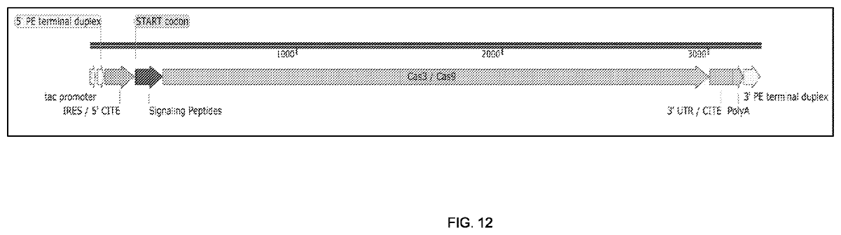

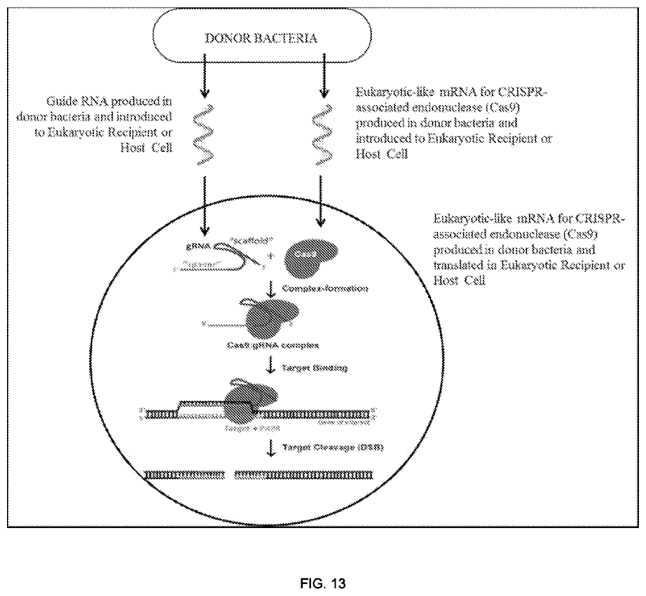

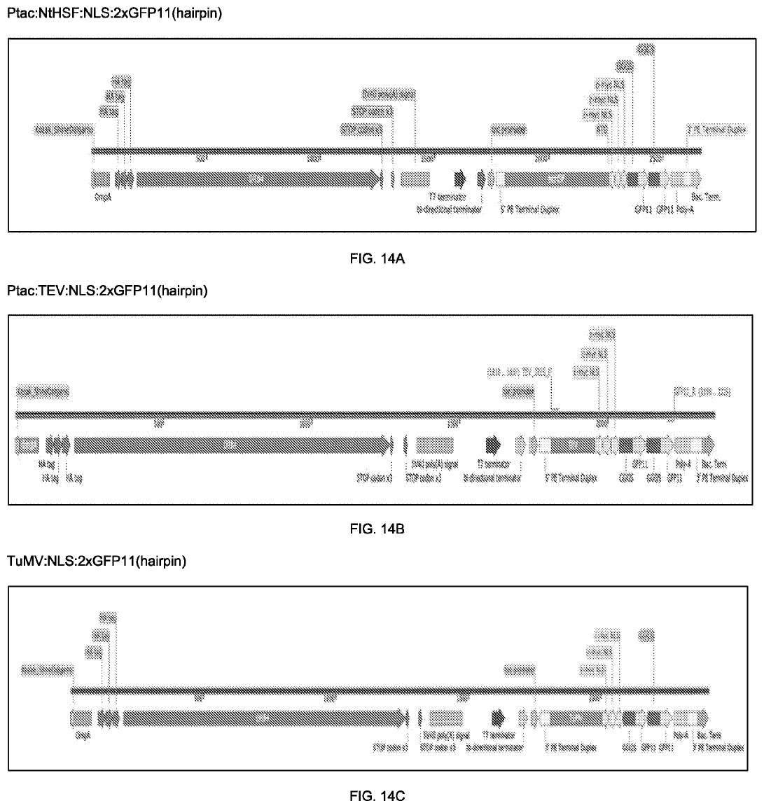

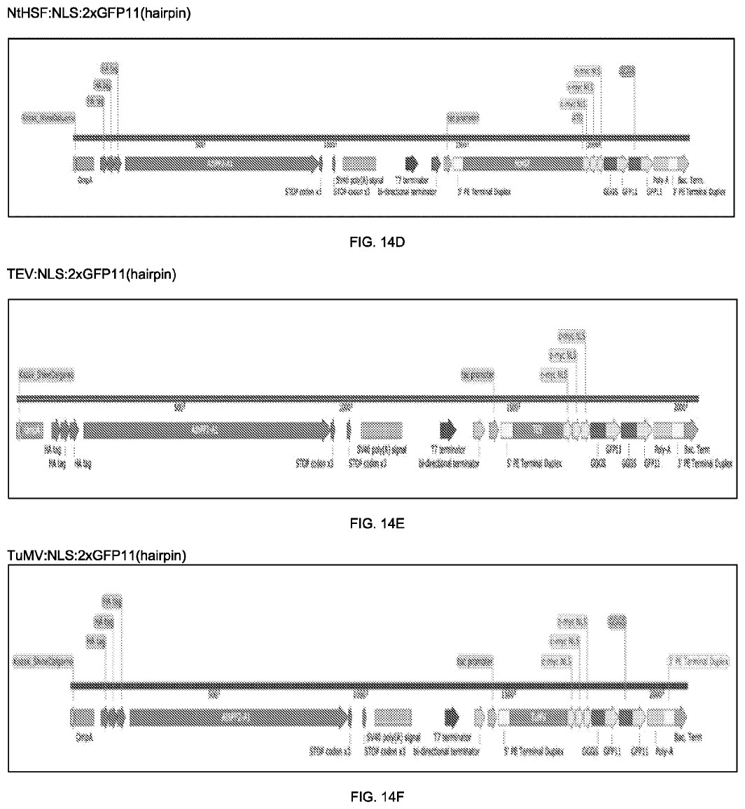

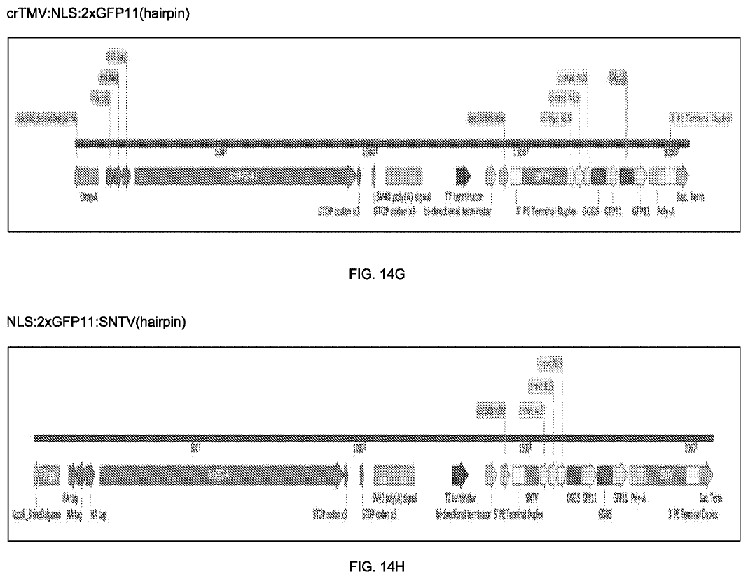

In this context, the gene-editing CRISPR/Cas9 technology generally encompasses an RNA-guided gene-editing platform that makes use of a bacterially derived protein (Cas9) and a synthetic gRNA to introduce a double-strand break at a specific location within the genome of the eukaryotic host. Generally, CRISPR/Cas9 may be used to generate a knock-out or disrupt target genes by co-expressing a gRNA specific to the gene to be targeted and the endonuclease Cas9. Specifically referring to FIG. 12, CRISPR may consist of two components: gRNA and a non-specific CRISPR-associated endonuclease (Cas9). The gRNA may be a short synthetic RNA composed of a scaffold sequence that may allow for Cas9-binding and a ˜20 nucleotide spacer or targeting sequence which defines the genomic target to be modified. In one preferred embodiment shown in FIG. 12, a donor prokaryotic organism, such as a bacterium, may be genetically modified to produce one or more gRNAs that are targeted to the genetic sequence of eukaryotic target gene and exported to a recipient eukaryotic host cell, for example through OMVs. In this embodiment, this genetically modified bacterium may also synthesize one or more eukaryotic-like mRNAs that may be introduced to a host or recipient eukaryotic cell and translated into a Cas9 endonuclease. As shown below, the gRNA and eukaryotic-like mRNA coding for a Cas9 endonuclease may initiate a transient genome editing function in the host cell.

In this embodiment, a donor prokaryotic organism, such as a bacterium, and preferably a symbiotic, endophytic, or probiotic bacterium, may be genetically modified to produce a eukaryotic-like mRNA for a Cas9 protein along with a specially designed guide RNA (gRNA) that directs the targeted DNA cut through hybridization with its matching genomic sequence. In this embodiment, it is possible to transiently introduce specific genetic alterations in one or more target genes in the donor eukaryotic host. In some embodiments, this transient CRISPR/Cas-9 system may be utilized to replace one or more existing wild-type genes with a modified version, while additional embodiments may include the addition of genetic elements that alter, reduce, increase or knock-out the expression of a target gene. In this embodiment, a target gene may include, but not be limited to, an endogenous gene, a transgene, or even a eukaryotic pathogen gene.

The inventive technology may include the generation of non-CRISPR transient gene editing systems. In one preferred embodiment, the invention may include a transient zinc finger, or zinc finger nuclease mediated genome editing system. The term “zinc finger,” as used herein, refers to a small nucleic acid-binding protein structural motif characterized by a fold and the coordination of one or more zinc ions that stabilize the fold. Zinc fingers encompass a wide variety of differing protein structures (see, e.g., Klug A, Rhodes D (1987). “Zinc fingers: a novel protein fold for nucleic acid recognition”. Cold Spring Harb. Symp. Quant. Biol. 52: 473-82, the entire contents of which are incorporated herein by reference).

Zinc fingers can be designed to bind a specific sequence of nucleotides, and zinc finger arrays comprising fusions of a series of zinc fingers, can be designed to bind virtually any desired target sequence. Such zinc finger arrays can form a binding domain of a protein, for example, of a nuclease, e.g., if conjugated to a nucleic acid cleavage domain. Different types of zinc finger motifs are known to those of skill in the art, including, but not limited to, Cys2His2, Gag knuckle, Treble clef, Zinc ribbon, Zn2/Cys6, and TAZ2 domain-like motifs (see, e.g., Krishna S S, Majumdar I, Grishin N V (January 2003). “Structural classification of zinc fingers: survey and summary”. Nucleic Acids Res. 31 (2): 532-50).

Typically, a single zinc finger motif binds 3 or 4 nucleotides of a nucleic acid molecule. Accordingly, a zinc finger domain comprising 2 zinc finger motifs may bind 6-8 nucleotides, a zinc finger domain comprising 3 zinc finger motifs may bind 9-12 nucleotides, a zinc finger domain comprising 4 zinc finger motifs may bind 12-16 nucleotides, and so forth. Any suitable protein engineering technique can be employed to alter the DNA-binding specificity of zinc fingers and/or design novel zinc finger fusions to bind virtually any desired target sequence from 3-30 nucleotides in length (see, e.g., Pabo C O, Peisach E, Grant R A (2001). “Design and selection of novel cys2H is2 Zinc finger proteins”. Annual Review of Biochemistry 70: 313-340; Jamieson A C, Miller J C, Pabo C O (2003). “Drug discovery with engineered zinc-finger proteins”. Nature Reviews Drug Discovery 2 (5): 361-368; and Liu Q, Segal D J, Ghiara J B, Barbas C F (May 1997). “Design of polydactyl zinc-finger proteins for unique addressing within complex genomes”. Proc. Natl. Acad. Sci. U.S.A. 94 (11); the entire contents of each of which are incorporated herein by reference).

Fusions between engineered zinc finger arrays and protein domains that cleave a nucleic acid can be used to generate a “zinc finger nuclease.” A zinc finger nuclease typically comprises a zinc finger domain that binds a specific target site within a nucleic acid molecule and a nucleic acid cleavage domain that cuts the nucleic acid molecule within or in proximity to the target site bound by the binding domain. Typical engineered zinc finger nucleases comprise a binding domain having between 3 and 6 individual zinc finger motifs and binding target sites ranging from 9 base pairs to 18 base pairs in length. Longer target sites are particularly attractive in situations where it is desired to bind and cleave a target site that is unique in a given genome.

As such, in a preferred embodiment a prokaryotic donor, such as a bacterium, may be genetically modified with a nucleic acid construct that may generate a eukaryotic-like mRNA that may be synthesized in the donor bacteria and then introduced to a eukaryotic host or recipient where it may be translated into a zinc finger nuclease directed to a target gene. Zinc finger nucleases can be generated to target a site of interest by methods well known to those of skill in the art. For example, zinc finger binding domains with a desired specificity can be designed by combining individual zinc finger motifs of known specificity. The structure of the zinc finger protein Zif268 bound to DNA has informed much of the work in this field and the concept of obtaining zinc fingers for each of the 64 possible base pair triplets and then mixing and matching these modular zinc fingers to design proteins with any desired sequence specificity has been described (Pavletich N P, Pabo Colo. (May 1991). “Zinc finger-DNA recognition: crystal structure of a Zif268-DNA complex at 2.1 A”. Science 252 (5007): 809-17, the entire contents of which are incorporated herein).

In some embodiments, separate zinc fingers may be generated that each recognizes a 3 base pair DNA sequence are combined to generate 3-, 4-, 5-, or 6-finger arrays that recognize target sites ranging from 9 base pairs to 18 base pairs in length. In some embodiments, longer arrays are contemplated. In other embodiments, 2-finger modules recognizing 6-8 nucleotides are combined to generate 4-, 6-, or 8-zinc finger arrays. In some embodiments, bacterial or phage display is employed to develop a zinc finger domain that recognizes a desired nucleic acid sequence, for example, a desired nuclease target site of 3-30 bp in length.

As noted above, zinc finger nucleases, in some embodiments may comprise a zinc finger binding domain and a cleavage domain fused or otherwise conjugated to each other via a linker, for example, a polypeptide spacer. The length of the linker determines the distance of the cut from the nucleic acid sequence bound by the zinc finger domain. If a shorter linker is used, the cleavage domain will cut the nucleic acid closer to the bound nucleic acid sequence, while a longer linker will result in a greater distance between the cut and the bound nucleic acid sequence. In some embodiments, the cleavage domain of a zinc finger nuclease has to dimerize in order to cut a bound nucleic acid. In some such embodiments, the dimer is a heterodimer of two monomers, each of which comprise a different zinc finger binding domain. For example, in some embodiments, the dimer may comprise one monomer comprising zinc finger domain A conjugated to a FokI cleavage domain, and one monomer comprising zinc finger domain B conjugated to a FokI cleavage domain. In this non-limiting example, zinc finger domain A binds a nucleic acid sequence on one side of the target site, zinc finger domain B binds a nucleic acid sequence on the other side of the target site, and the dimerize FokI domain cuts the nucleic acid in between the zinc finger domain binding sites.

The inventive technology may include the generation of a transient TALEN gene editing system. The term TALEN or “Transcriptional Activator-Like Element Nuclease” or “TALE nuclease” as used herein, refers to an artificial nuclease comprising a transcriptional activator like effector DNA binding domain to a DNA cleavage domain, for example, a FokI domain. A number of modular assembly schemes for generating engineered TALE constructs have been reported (Zhang, Feng; et. al. (February 2011). “Efficient construction of sequence-specific TAL effectors for modulating mammalian transcription”. Nature Biotechnology 29 (2): 149-53; Geibler, R.; Scholze, H.; Hahn, S.; Streubel, J.; Bonas, U.; Behrens, S. E.; Boch, J. (2011), Shiu, Shin-Han. ed. “Transcriptional Activators of Human Genes with Programmable DNA-Specificity”. PLoS ONE 6 (5): e19509; Cermak, T.; Doyle, E. L.; Christian, M.; Wang, L.; Zhang, Y.; Schmidt, C.; Baller, J. A.; Somia, N. V. et al. (2011). “Efficient design and assembly of custom TALEN and other TAL effector-based constructs for DNA targeting”. Nucleic Acids Research; Morbitzer, R.; Elsaesser, J.; Hausner, J.; Lahaye, T. (2011). “Assembly of custom TALE-type DNA binding domains by modular cloning”. Nucleic Acids Research; Li, T.; Huang, S.; Zhao, X.; Wright, D. A.; Carpenter, S.; Spalding, M. H.; Weeks, D. P.; Yang, B. (2011). “Modularly assembled designer TAL effector nucleases for targeted gene knockout and gene replacement in eukaryotes”. Nucleic Acids Research; Weber, E.; Gruetzner, R.; Werner, S.; Engler, C.; Marillonnet, S. (2011). Bendahmane, Mohammed. ed. “Assembly of Designer TAL Effectors by Golden Gate Cloning”. PLoS ONE 6 (5): e19722; each of which is incorporated herein by reference).

Those of skill in the art will understand that TALE nucleases can be engineered to target virtually any genomic sequence with high specificity, and that such engineered nucleases can be used in embodiments of the present technology to manipulate the genome of a cell, e.g., by delivering the respective TALEN via a method or strategy disclosed herein under circumstances suitable for the TALEN to bind and cleave its target sequence within the genome of the cell. In some embodiments, the delivered TALEN targets a gene or allele associated with a disease or disorder or a biological process, or one or more target genes. In some embodiments, delivery of the TALEN to a subject confers a therapeutic benefit to the subject, such as reducing, ameliorating or eliminating the disease condition in a patient.

In this embodiment, a donor prokaryotic organism, such as a bacterium, may be genetically modified to produce a eukaryotic-like TALE nuclease configured to affect a gene editing function on a target gene in a eukaryotic host. In this embodiment, it is possible to transiently introduce specific genetic alterations in one or more target genes in the donor eukaryotic host. In some embodiments, this transient TALEN system may be utilized to replace one or more existing wild-type genes with a modified version, while additional embodiments may include the addition of genetic elements that alter, reduce, increase, or knock-out the expression of a target gene. In this embodiment, a target gene may include, but not be limited to, an endogenous gene, a transgene, or even a eukaryotic pathogen gene.

In some embodiments, the target gene of a cell, tissue, organ, or organism is altered by a nuclease delivered to the cell via a strategy or method disclosed herein, e.g., CRISPR/cas-9, a TALEN, or a zinc-finger nuclease, or a plurality or combination of such nucleases. In some embodiments, a single- or double-strand break is introduced at a specific site within the genome by the nuclease, resulting in a disruption of the target genomic sequence.

The inventive technology may include the generation of a transient gene editing system that may be used to treat a disease condition in a eukaryotic organism, and in particular a human or plant. In this embodiment, a eukaryotic-like mRNAs may be generated and synthesized in a prokaryotic bacterium. In this embodiment, the prokaryotic bacteria may include bacteria that naturally colonize a human patient or plant, such as bacteria naturally found in the patient's or plants normal microbiome. Examples may include various symbiotic, endosymbiotic, probiotic, and/or enteric as well as endophytic bacterial strains. In this preferred embodiment, a therapeutically effective amount of donor prokaryotic bacteria may be administered to a patient or plant. These donor prokaryotic bacteria may synthesize eukaryotic-like mRNA which may be introduced to the host patient or plant where they are translated into a target protein. As noted above, such eukaryotic-like mRNAs may encode one or more target proteins that provide a metabolic advantage, or correct a metabolic deficiency in the human patient/plant, and/or performs a gene editing function. More broadly, such eukaryotic-like mRNAs may encode a target protein that ameliorate, or treat a disease condition. For example, in certain embodiment, a eukaryotic-like mRNA may be translated in the host to generate a protein-based vaccine or other prophylactic disease preventative agent. In other embodiments, the invention may include a human transient gene editing system as generally described herein. In certain embodiments, a eukaryotic-like mRNA may be translated in the host to generate a phenotypic, biochemical or metabolic change in the host. Such changes may generally be associated with the amelioration of a disease condition.

Each of the aforementioned systems nay be embodied in genetic constructs that may include transcription regulation elements such as promoters, terminators, co-activators and co-repressors and other control elements. Such systems may allow for control of the type, timing and amount of, eukaryotic-like RNA molecules or other proteins, expressed or transported within the system. Another embodiment of the present invention may include a cell comprising the isolated nucleic acid agent, such as an expression vector coding a eukaryotic-like mRNA that may be expressed in a prokaryotic cells. Such an expression vector may include a nucleic acid construct, such as a plasmid. The present invention may further include a cell comprising the isolated nucleic acid agent, or the nucleic acid construct, such as a plasmid. The present invention may further include a cell having an eukaryotic-like mRNA, or the translational product of a eukaryotic-like mRNA.

Some embodiments of the invention that may further include the co-expression of one or more “helper” genes that may aid in eukaryotic-like mRNA expression, protection, or secretion and the like. Another aim of the present invention may include the use of an autotrophic bacteria, as well as an RNase III deficient strains of bacteria as a nucleic acid agent transmission vector.

In certain embodiment, donor bacterium may include one or more endophytes. Plants may harbor a number of beneficial bacteria intracellularly as well as on their surfaces, including roots, leaves, and stem tissues. Endophytic and ectopic bacteria that live in association with plants include those in the following subphyla: Acido bacteria, Actinobacteria, Alphaproteo bacteria, Armatimonadetes, Bacteroides, Betaproteobacteria, Deltaproteobacteria, Firmicutes, Grammaproteobacteria, TM7, Bacillus, and Escherichia among others. In certain embodiment, the invention may include one or more genetically modified endophyte bacteria having suppressed RNaseIII activity. Specific examples may further include Bacillus subtilis CCB422, E. coli HT27, or HT115, JC8031 as described by Sayre et al. in PCT/US2017/064977, which is incorporated herein by reference.

Examples of eukaryotic plants may include both monocot and diocot plants. Example plants that may be recipient eukaryotic hosts may be selected from the group consisting of: grains, corn, wheat, rice, barley, oats, sorghum, millet, sunflower, safflower, cotton, soy, canola, alfalfa, Arabidopsis, cannabis, potato, Brassica, peanut, tobacco, tropical fruits and flowers, banana, duckweed, gladiolus, sugar cane, pineapples, dates, onions, pineapple, cashews, pistachios, flowers, ornamentals, conifers, deciduous, grapes, citrus, roses, apples, peaches, strawberries, almonds, coffee, oaks, beans, legumes, watermelon, squashes, cabbage, turnip, mustard, cacti, pecans, flax, sweet potato, soybean, coconut, avocado, beets, cantaloupe, Cannabis, hemp. and vegetables.

Certain embodiments of the invention include isolated eukaryotic-like RNAs. The terms “isolated,” “purified,” or “biologically pure” as used herein, refer to material that is substantially or essentially free from components that normally accompany the material in its native state or when the material is produced. In an exemplary embodiment, purity and homogeneity are determined using analytical chemistry techniques such as polyacrylamide gel electrophoresis or high performance liquid chromatography. A nucleic acid or particular bacteria that are the predominant species present in a preparation is substantially purified. In an exemplary embodiment, the term “purified” denotes that a nucleic acid or protein that gives rise to essentially one band in an electrophoretic gel. Typically, isolated nucleic acids or proteins have a level of purity expressed as a range. The lower end of the range of purity for the component is about 60%, about 70%, or about 80%, and the upper end of the range of purity is about 70%, about 80%, about 90%, or more than about 90%.

The term “contact” or “introduce” with an a recipient or host eukaryotic organism or cell as well as the term “contact with” or “uptake by” an eukaryotic organism with regard to a nucleic acid molecule, such as a eukaryotic-like RNA, includes internalization of the nucleic acid molecule into the organism, for example and without limitation: ingestion of the molecule by the organism (e.g., by feeding); contacting the organism with a composition comprising the nucleic acid molecule; soaking of organisms with a solution comprising the nucleic acid molecule; injecting the organism with a composition comprising the nucleic acid molecule; and spraying the organism with an aerosol composition comprising the nucleic acid molecule. Such terms generally encompass any physical or temporal contacting of a host cell with a eukaryotic-like RNA.

The term “expression,” as used herein, or “expression of a coding sequence” (for example, a gene or a transgene) refers to the process by which the coded information of a nucleic acid transcriptional unit (including, e.g., genomic DNA or cDNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s).

The term “nucleic acid” or “nucleic acid molecules” include single- and double-stranded forms of DNA; single-stranded forms of RNA; and double-stranded forms of RNA (dsRNA). The term “nucleotide sequence” or “nucleic acid sequence” refers to both the sense and antisense strands of a nucleic acid as either individual single strands or in the duplex. The term “ribonucleic acid” (RNA) is inclusive of iRNA (inhibitory RNA), dsRNA (double stranded RNA), siRNA (small interfering RNA), mRNA (messenger RNA), miRNA (microRNA), hpRNA (hairpin RNA), tRNA (transfer RNA), whether charged or discharged with a corresponding acylated amino acid), and cRNA (complementary RNA). The term “deoxyribonucleic acid” (DNA) is inclusive of cDNA, genomic DNA, and DNA-RNA hybrids.

The terms “nucleic acid segment” and “nucleotide sequence segment,” or more generally “segment,” will be understood by those in the art as a functional term that includes both genomic sequences, ribosomal RNA sequences, transfer RNA sequences, messenger RNA sequences, operon sequences, and smaller engineered nucleotide sequences that encoded or may be adapted to encode, peptides, polypeptides, or proteins.

The term “prokaryotic” is meant to include all bacteria, archaea, and/or cyanobacteria which can be transformed or transfected with a nucleic acid and express a eukaryotic-like RNA of the invention. Prokaryotic hosts may include gram negative as well as gram positive bacteria. The term “eukaryotic” is meant to include yeast, algae, plants, higher plants, insect, and mammalian cells.

The term “gene” or “sequence” refers to a coding region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (down-stream) the coding region (open reading frame, ORF) as well as, where applicable, intervening sequences (i.e., introns) between individual coding regions (i.e., exons). The term “structural gene” as used herein is intended to mean a DNA sequence that is transcribed into mRNA which is then translated into a sequence of amino acids characteristic of a specific polypeptide.

A nucleic acid molecule may include either or both naturally occurring and modified nucleotides linked together by naturally occurring and/or non-naturally occurring nucleotide linkages. Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications (e.g., uncharged linkages: for example, methyl phosphonates, phosphotriesters, phosphoramidates, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages: for example, alpha anomeric nucleic acids, etc.). The term “nucleic acid molecule” also includes any topological conformation, including single-stranded, double-stranded, partially duplexed, triplexed, hair-pinned, circular, and padlocked conformations.

The term “target gene” or “coding region” as used herein is intended to mean a DNA sequence that is transcribed into mRNA which is then translated into a sequence of amino acids characteristic of a specific polypeptide.

As used herein with respect to DNA, the term “coding sequence,” “structural nucleotide sequence,” or “structural nucleic acid molecule” refers to a nucleotide sequence that is ultimately translated into a polypeptide, via transcription and mRNA, when placed under the control of appropriate regulatory sequences. With respect to RNA, the term “coding sequence” refers to a nucleotide sequence that is translated into a peptide, polypeptide, or protein. The boundaries of a coding sequence are determined by a translation start codon at the 5′-terminus and a translation stop codon at the 3′-terminus. Coding sequences include, but are not limited to: genomic DNA; cDNA; EST; and recombinant nucleotide sequences.

The term “sequence identity” or “identity,” as used herein in the context of two nucleic acid or polypeptide sequences, refers to the residues in the two sequences that are the same when aligned for maximum correspondence over a specified comparison window.

The term “recombinant” when used with reference, e.g., to a cell, or nucleic acid, protein, or vector, indicates that the cell, organism, nucleic acid, protein or vector, has been modified by the introduction of a heterologous nucleic acid or protein, or the alteration of a native nucleic acid or protein, or that the cell is derived from a cell so modified. Thus, for example, recombinant cells may express genes that are not found within the native (nonrecombinant or wild-type) form of the cell or express native genes that are otherwise abnormally expressed—over-expressed, under expressed or not expressed at all.

As used herein, “heterologous” or “exogenous” in reference to a nucleic acid is a nucleic acid that originates from a foreign species, or is synthetically designed, or, if from the same species, is substantially modified from its native form in composition and/or genomic locus by deliberate human intervention. A heterologous protein may originate from a foreign species or, if from the same species, is substantially modified from its original form by deliberate human intervention. By “host cell” is meant a cell which contains an introduced nucleic acid construct and supports the replication and/or expression of the construct.

As used herein, the term “genome” refers to chromosomal DNA found within the nucleus of a cell, and also refers to organelle DNA found within subcellular components of the cell. The term “genome” as it applies to bacteria refers to both the chromosome and plasmids within the bacterial cell. In some embodiments of the invention, a DNA molecule may be introduced into a bacterium such that the DNA molecule is integrated into the genome of the bacterium. In these and further embodiments, the DNA molecule may be either chromosomally-integrated or located as or in a stable plasmid.

The term, “operably linked,” when used in reference to a regulatory sequence and a coding sequence, means that the regulatory sequence affects the expression of the linked coding sequence. “Regulatory sequences,” or “control elements,” refer to nucleotide sequences that facilitate the transcription of eukaryotic-like mRNAs in prokaryotic cells, and/or facilitate the export of eukaryotic-like mRNAs out of a prokaryotic cells, and/or facilitate the up take of eukaryotic-like mRNAs by eukaryotic cells, and/or facilitate the translation of eukaryotic-like mRNAs in eukaryotic cells. The terms may additionally encompass nucleotide sequences that influence the timing and level/amount of transcription, RNA processing or stability, or translation of the associated coding sequence. Regulatory sequences may include promoters; translation leader sequences; introns; enhancers; stem-loop structures; repressor binding sequences; termination sequences; polyadenylation recognition sequences and the like. Particular regulatory sequences may be located upstream and/or downstream of a coding sequence operably linked thereto. Also, particular regulatory sequences operably linked to a coding sequence may be located on the associated complementary strand of a double-stranded nucleic acid molecule.

As used herein, the term “promoter” refers to a region of DNA that may be upstream from the start of transcription, and that may be involved in recognition and binding of RNA polymerase and other proteins to initiate transcription. A promoter may be operably linked to a coding sequence for expression in a cell, or a promoter may be operably linked to a nucleotide sequence encoding a signal sequence which may be operably linked to a coding sequence for expression in a cell. A “plant promoter” may be a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred.” Promoters which initiate transcription only in certain tissues are referred to as “tissue-specific.”

A “cell type-specific” promoter primarily drives expression in certain cell types in one or more organs, for example, vascular cells in roots or leaves. An “inducible” promoter may be a promoter which may be under environmental control. Examples of environmental conditions that may initiate transcription by inducible promoters include anaerobic conditions and the presence of light. Tissue-specific, tissue-preferred, cell type specific, and inducible promoters constitute the class of “non-constitutive” promoters. A “constitutive” promoter is a promoter which may be active under most environmental conditions or in most cell or tissue types.

The term “vector” refers to some means by which DNA, RNA, a protein, or polypeptide can be introduced into a host. The polynucleotides, protein, and polypeptide which are to be introduced into a host can be therapeutic or prophylactic in nature; can encode or be an antigen; can be regulatory in nature, etc. There are various types of vectors including virus, plasmid, bacteriophages, cosmids, and bacteria.

An “expression vector” is nucleic acid capable of replicating in a selected host cell or organism. An expression vector can replicate as an autonomous structure, or alternatively can integrate, in whole or in part, into the host cell chromosomes or the nucleic acids of an organelle, or it is used as a shuttle for delivering foreign DNA to cells, and thus replicate along with the host cell genome. Thus, an expression vector are polynucleotides capable of replicating in a selected host cell, organelle, or organism, e.g., a plasmid, virus, artificial chromosome, nucleic acid fragment, and for which certain genes on the expression vector (including genes of interest) are transcribed and translated into a polypeptide or protein within the cell, organelle or organism; or any suitable construct known in the art, which comprises an “expression cassette.” In contrast, as described in the examples herein, a “cassette” is a polynucleotide containing a section of an expression vector of this invention. The use of the cassettes assists in the assembly of the expression vectors. An expression vector is a replicon, such as plasmid, phage, virus, chimeric virus, or cosmid, and which contains the desired polynucleotide sequence operably linked to the expression control sequence(s).

As is known in the art, different organisms preferentially utilize different codons for generating polypeptides. Such “codon usage” preferences may be used in the design of nucleic acid molecules encoding the proteins and chimeras of the invention in order to optimize expression in a particular host cell system.

A polynucleotide sequence is operably linked to an expression control sequence(s) (e.g., a promoter and, optionally, an enhancer) when the expression control sequence controls and regulates the transcription and/or translation of that polynucleotide sequence.

Unless otherwise indicated, a particular nucleic acid sequence also implicitly encompasses conservatively modified variants thereof (e.g., degenerate codon substitutions), the complementary (or complement) sequence, and the reverse complement sequence, as well as the sequence explicitly indicated. Specifically, degenerate codon substitutions may be achieved by generating sequences in which the third position of one or more selected (or all) codons is substituted with mixed-base and/or deoxyinosine residues (see e.g., Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). Because of the degeneracy of nucleic acid codons, one can use various different polynucleotides to encode identical polypeptides. Table 1a, infra, contains information about which nucleic acid codons encode which amino acids.

TABLE 4
Amino acid Nucleic acid codons

| Amino Acid | Nucleic Acid Codons |
|---|---|
| Ala/A | GCT, GCC, GCA, GCG |
| Arg/R | CGT, CGC, CGA, CGG, AGA, |
| | AGG |
| Asn/N | AAT, AAC |
| Asp/D | GAT, GAC |
| Cys/C | TGT, TGC |
| Gln/Q | CAA, CAG |
| Glu/E | GAA, GAG |
| Gly/G | GGT, GGC, GGA, GGG |
| His/H | CAT, CAC |
| Ile/I | ATT, ATC, ATA |
| Leu/L | TTA, TTG, CTT, CTC, CTA, CTG |
| Lys/K | AAA, AAG |
| Met/M | ATG |
| Phe/F | TTT, TTC |
| Pro/P | CCT, CCC, CCA, CCG |
| Ser/S | TCT, TCC, TCA, TCG, AGT, AGC |
| Thr/T | ACT, ACC, ACA, ACG |
| Trp/W | TGG |
| Tyr/Y | TAT, TAC |
| Val/V | GTT, GTC, GTA, GTG |

The term “plant” or “plant system” includes whole plants, plant organs, progeny of whole plants or plant organs, embryos, somatic embryos, embryo-like structures, protocorms, protocorm-like bodies (PLBs), and culture and/or suspensions of plant cells. Plant organs comprise, e.g., shoot vegetative organs/structures (e.g., leaves, stems and tubers), roots, flowers and floral organs/structures (e.g., bracts, sepals, petals, stamens, carpels, anthers and ovules), seed (including embryo, endosperm, and seed coat) and fruit (the mature ovary), plant tissue (e.g., vascular tissue, ground tissue, and the like) and cells (e.g., guard cells, egg cells, trichomes and the like). The invention may also include Cannabaceae and other Cannabis strains, such as C. sativa generally.

Any inducible promoter can be used in some embodiments of the invention. See Ward et al. (1993) Plant Mol. Biol. 22:361-366. With an inducible promoter, the rate of transcription increases in response to an inducing agent. Exemplary inducible promoters include, but are not limited to: Promoters from the ACEI system that responds to copper; In2 gene from maize that responds to benzenesulfonamide herbicide safeners; Tet repressor from Tn10; and the inducible promoter from a steroid hormone gene, the transcriptional activity of which may be induced by a glucocorticosteroid hormone are general examples (Schena et al. (1991) Proc. Natl. Acad. Sci. USA 88:0421).

Any inducible promoter can be used in some embodiments of the invention. See Ward et al. (1993) Plant Mol. Biol. 22:361-366. With an inducible promoter, the rate of transcription increases in response to an inducing agent. Exemplary inducible promoters include, but are not limited to: Promoters from the ACEI system that responds to copper; In2 gene from maize that responds to benzenesulfonamide herbicide safeners; Tet repressor from Tn10; and the inducible promoter from a steroid hormone gene, the transcriptional activity of which may be induced by a glucocorticosteroid hormone are general examples (Schena et al. (1991) Proc. Natl. Acad. Sci. USA 88:0421).

As used herein, the terms “transformation” or “genetically modified” refers to the transfer of one or more nucleic acid molecule(s) into a cell. A microorganism is “transformed” or “genetically modified” by a nucleic acid molecule transduced into the bacteria when the nucleic acid molecule becomes stably replicated by the bacteria. As used herein, the term “transformation” or “genetically modified” encompasses all techniques by which a nucleic acid molecule can be introduced into a prokaryotic donor cell.

An “expression vector” is nucleic acid capable of replicating in a selected host cell or organism. An expression vector can replicate as an autonomous structure, or alternatively can integrate, in whole or in part, into the host cell chromosomes or the nucleic acids of an organelle, or it is used as a shuttle for delivering foreign DNA to cells, and thus replicate along with the host cell genome. Thus, an expression vector are polynucleotides capable of replicating in a selected host cell, organelle, or organism, e.g., a plasmid, virus, artificial chromosome, nucleic acid fragment, and for which certain genes on the expression vector (including genes of interest) are transcribed and translated into a polypeptide or protein within the cell, organelle or organism; or any suitable construct known in the art, which comprises an “expression cassette.” In contrast, as described in the examples herein, a “cassette” is a polynucleotide containing a section of an expression vector of this invention. The use of the cassettes assists in the assembly of the expression vectors. An expression vector is a replicon, such as plasmid, phage, virus, chimeric virus, or cosmid, and which contains the desired polynucleotide sequence operably linked to the expression control sequence(s).

A polynucleotide sequence is operably linked to an expression control sequence(s) (e.g., a promoter and, optionally, an enhancer) when the expression control sequence controls and regulates the transcription and/or translation of that polynucleotide sequence.

The term “recombinant” when used with reference, e.g., to a cell, or nucleic acid, protein, or vector, indicates that the cell, organism, nucleic acid, protein or vector, has been modified by the introduction of a heterologous nucleic acid or protein, or the alteration of a native nucleic acid or protein, or that the cell is derived from a cell so modified. Thus, for example, recombinant cells may express genes that are not found within the native (nonrecombinant or wild-type) form of the cell or express native genes that are otherwise abnormally expressed—over-expressed, under expressed or not expressed at all.

The terms “approximately” and “about” refer to a quantity, level, value or amount that varies by as much as 30%, or in another embodiment by as much as 20%, and in a third embodiment by as much as 10% to a reference quantity, level, value or amount. As used herein, the singular form “a,” “an,” and “the” include plural references unless the context clearly dictates otherwise. For example, the term “a bacterium” includes both a single bacterium and a plurality of bacteria.

As used herein the term “method” and/or “system: refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

As used herein, “symbiotic” or “symbionts” generally refer to a bacterium that is a symbiont of a eukaryotic host. It may also include bacteria that persist throughout the life-cycle of a eukaryotic host, either internally or externally, and may further be passed horizontally to a eukaryotic host. Endosymbionts generally refers to a subgroup of symbionts. The term “endophyte” or endophytic” refer to a bacterium that is a symbiont of a plant host.

The term “probiotic” refers to a microorganism, such as bacteria, that may colonize a host for a sufficient length of time to delver a therapeutic or effective amount of eukaryotic-like RNA polynucleotide. A probiotic may include endosymbiotic bacteria, or naturally occurring flora that may permanently to temporarily colonize a eukaryotic organism. Probiotic organisms may also include algae, and fungi, such as yeast.

In one embodiment, a eukaryote may be a an animal, and preferably a mammal, and more preferably a human. In another embodiment, a eukaryote may include a “aquatic organisms” and/or “aquatic animal” as used herein include organisms grown in water, either fresh or saltwater. Aquatic organisms/animals includes vertebrates, invertebrates, arthropods, fish, mollusks, including, shrimp (e.g., penaeid shrimp, Penaeus esculentu, Penaeus setiferus, Penaeus stylirostris, Penaeus occidentalis, Penaeus japonicus, Penaeus vannamei, Penaeus monodon, Penaeus chinensis, Penaeus aztecus, Penaeus duorarum, Penaeus indicus, and Penaeus merguiensis, Penaeus calif orniensis, Penaeus semisulcatus, Penaeus monodon, brine shrimp, freshwater shrimp, etc), crabs, oysters, scallop, prawn clams, cartilaginous fish (e.g., sea bream, trout, bass, striped bass, tilapia, catfish, salmonids, carp, catfish, yellowtail, carp zebrafish, red drum, etc), crustaceans, among others. Shrimp include, shrimp raised in aquaculture as well.

As used herein, the term “eukaryotic-like RNA” or “eukaryotic-like mRNA” refers to a RNA molecule expressed in a prokaryotic or other non-eukaryotic systems that is competent to be expressed in a recipient eukaryotic cell.

Notably, all DNA sequences provided may encompass all RNA and amino acid sequences, and vice versa as would be ascertainable by those of ordinary skill in the art, for example through Uracil substitutions as well as redundant codons. Additionally, all sequences include codon-optimized embodiments as would be ascertainable by those of ordinary skill in the art. As such, the term “encoding” or “coding sequence” or “coding” means both encoding a nucleotide and/or amino acid sequence and vice versa.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

### Example 1: Design and Expression of RNAs in Prokaryotic Systems that are Competent for Eukaryotic Ribosome Binding and Competent for Translation Once Imported into Eukaryotic Host Cells

The present inventors demonstrate the design and production of messenger RNAs (mRNAs) in prokaryotic systems that are competent for eukaryotic ribosome binding and translation once imported into eukaryotic host cells. As generally shown in FIGS. 1-3, in one preferred embodiment the inventors generated a plurality of exemplary bacterially produced Euk-mRNA constructs that include one or more coding region features that may be required, or facilitate eukaryotic translation. Such nucleotide constructs may form expression vectors that may be used to transform one or more bacteria. Such nucleotide constructs may further be expressed in a genetically modified prokaryotic cell, such as bacteria or more preferably a symbiotic, endosymbiotic or endophytic bacteria, generating a eukaryotic-like mRNA molecule that may be transported to a eukaryotic host cell where it may be competently translated.

As generally shown in FIGS. 1-2, in one embodiment the inventors generated a nucleotide constructs configured to express eukaryotic-like mRNA molecule having a modified 5′ end region. Specifically, in one embodiment, at the 5′ end of the coding region, the inventors included a 5′ untranslated region (UTR) sequence to generate a 5′-CAP independent translation machinery recruitment platform. In this embodiment, the 5′ untranslated region (UTR) may allow recruitment of translation machinery in the absence of a 5′-CAP. In contrast to eukaryotic mRNAs, prokaryote mRNAs are not capped. As further shown in the figures, a plurality of 5′-UTRs sequences were constructed, designated herein as Internal Ribosome Entry Sites (IRES). IRES secondary structure is generally sufficient for recruitment of ribosome subunits and can be found in viral as well as plant genomes. In addition, to avoid translation of the mRNA in the bacteria, all prokaryote ribosome binding sites were omitted from the coding region.

As an alternative to the use of IRES to substitute for eukaryotic mRNA 5′ capping, the present inventors generated constructs configured to express eukaryotic-like mRNA molecule having “Cap independent translation element” (CITE) sequences. CITEs are generally composed of 5′ and 3′ UTR structured RNA sequences that allow interaction between both ends of the coding RNA replicating the effect of interaction between Poly-A binding proteins (PABP) and translation initiation factors (eIF4G). Furthermore, since prokaryotes do not post-transcriptionally poly-adenylate its the 3′ UTR of mRNAs, a poly-A tail was added to the design of the coding region 3′ UTR of the mRNA to facilitate PABP binding, thus protecting RNA from degradation and allowing its translation in eukaryotic cells.

### Example 2: Design and Expression of a Split Protein Expression System to Confirm Transcription of Eukaryotic-Like mRNA in a Prokaryotic Cell which is Taken Up and Translated in a Eukaryotic Cell

As generally shown in FIGS. 1A-E and 2A-J, in exemplary embodiments, the coding regions of the mRNA constructs encode two GFP11 ß-sheets in tandem separated by a linker sequence. Notably, GFP proteins only fluoresce if its ß-barrel, composed of eleven ß-sheets, is complete. By only encoding a part of the GFP protein in the mRNA produced in prokaryotic cells, and testing the exemplary system by expressing the remaining GFP protein (GFP1-10) in eukaryotic cells—in this embodiment plant cells, as part of a split GFP approach, the inventors may demonstrate that its system that will only yield fluorescence from GFP if the prokaryotic transcribed eukaryotic-like RNA encoding GFP11 is synthesized in the prokaryote, transported and delivered to the eukaryote and translated by the eukaryote into protein that are expressing GFP1-10. The translated GFP11 protein associates with the GFP1-10 protein to then reconstitute the GFP ß-barrel allowing for GFP fluorescence. Finally, by introducing a nuclear localization signal (NLS) in the coding sequence we are able to restrict fluorescence signals to the nuclear compartment, a further test of protein function achieved by reconstituting the split-GFP in the nucleus.

### Example 3: Validation of Expression of Eukaryotic mRNA Design in a Eukaryote

All exemplary gene cassettes were synthesized in vitro and sub-cloned by the inventors into modified plasmid backbones. For in vivo expression validation of construct design in transformed plants, both the linear (FIG. 1) and hairpin forms (FIG. 2), IRES and CITE UTR regions associated with NLS:2×GFP11 coding sequence were cloned into modified pEARLEYGATE301 plant transformation vectors, such as, for example of an Agrobacterium Ti-plasmid plant transformation vector. The inventors further removed Gateway recombination sites (attR) and a 35S (cauliflower mosaic virus) CaMV promoter was introduced into the plasmid backbone. Eukaryotic mRNA (sometime referred to as Euk-mRNA) coding sequences were subsequently cloned downstream of 35S+1 nucleotide gene promoter so that the mRNA transcript will only include IRES or CITE sequences in its 5′UTR, or in the case of hairpin constructs, have the RNA pairing sequence as its upstream 5′-UTR. These plasmids were subsequently introduced into Agrobacterium tumefaciens GV3101 and used to transform plants expressing GFP10.

### Example 4: Expression of Euk-mRNA Sequences Encoding GFP11 in Bacteria for Delivery to and Expression in Plants Expressing GFP10

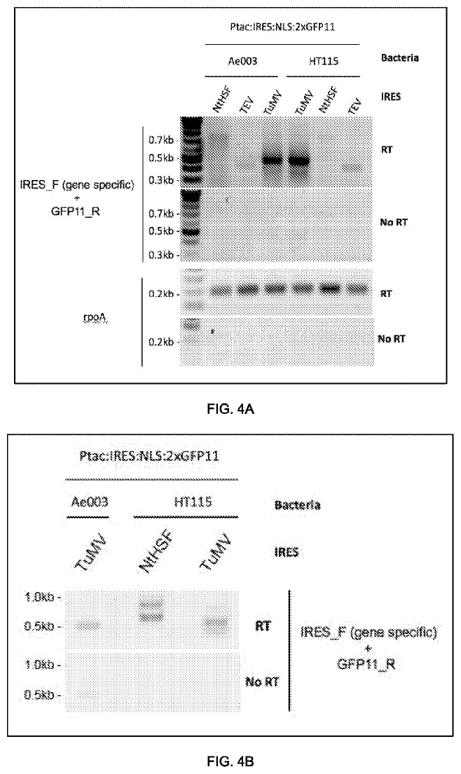

For prokaryotic expression of IRES and CITE hairpin GFP11 constructs, the various NLS:2×GFP11 coding sequences (FIG. 2) were cloned under Ptac promoter sequence immediately downstream of the transcription initiation site (+1) (The Tac-Promoter (abbreviated as Ptac), or tac vector is a synthetically produced DNA promoter, produced from the combination of promoters from the trp and lac operons). Plasmid constructs were subsequently transformed into E. coli HT115 Δrnc (as described in U.S. Ser. No. 16/031,607, such description being incorporated herein by reference) and Enterobacter cloacae WT mCherry and Δrnc strains developed by Applicant and transcription of gene constructs validated by RT-PCR (FIG. 4). Additionally, RNA was extracted from bacterial produced outer membrane vesicles, isolated following 24 hour incubation of 50 ml liquid cultures, and as presented in FIG. 4B. TuMV:NLS:2×GFP11 and NtHSF transcripts were identified in bacterial OMVs. As demonstrated by Sayre et al., in PCT/US2017/064977 (incorporated herein by reference), OMVs have previously been shown to transport RNA molecules to eukaryotic hosts.

### Example 5: In Planta Validation of Eukaryotic-Like mRNA Design and Function

To verify that introduction of IRES and CITE sequences does not interfere with correct translation of coding region of the NLS:2×GFP11 peptide, the present inventors performed a set of transient assays of Euk-mRNA-GFP11 in planta. These assays consisted of co-infiltration in Nicotiana benthamiana leaves with a Agrobacterium GV3101 strain transformed with either: 1) h Agrobacterium Ti-plasmid encoding a positive control vector (UBQ:NLS:GFP11:mCherry) encoding GFP11 and expressing mCherry fluorescent protein, 2) an Agrobacterium plant transformation vector (GV3101) encoding GFP1-10 tagged with a nuclear localization signal (NLS) expressed under the control of a ubiquitin promoter (UBQ); or 3) an Agrobacterium plant transformation vector GV3101 encoding linear IRES or CITE: NLS:2×GFP11 constructs (lacking the hairpin forming 5′ UTR sequence motif—see FIG. 1).

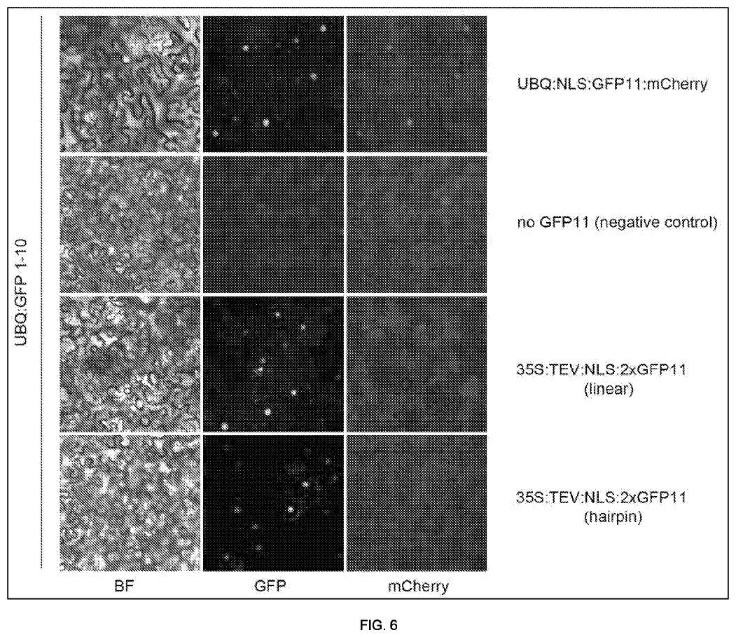

As shown in FIGS. 5-6, infiltration of UBQ:NLS:GFP11:mCherry and NLS:GFP1-10 alone (Mock), respectively, did not yield GFP fluorescence—green—whereas in leaves co-infiltrated with NLS:GFP1-10 with NLS:GFP11:mCherry (positive control) GFP positive nuclei were readily identified. In samples where IRES/CITE:NLS:2×GFP11 was co-infiltrated with NLS:GFP1-10 GFP positive nuclei were also observed indicating that construct design is competent to drive translation of NLS:2×GFP11 in eukaryotic systems.

The present inventors next sought to demonstrate that the addition of the hairpin forming 5′ UTR sequence motif (FIG. 2, 3) that is predicted to protect RNA from degradation mediated by RNAse exo-nucleases and help conserve poly-A integrity would allow for Euk-mRNA translation. The terminal hairpin region can also serve as a docking point to dsRNA binding proteins (helper proteins) that can potentially help traffic RNA from prokaryotic cells to eukaryotic hosts by targeting helper proteins to periplasm of, for example, gram negative organisms and facilitate inclusion of eukaryotic-like RNA into outer membrane vesicles (OMVs).

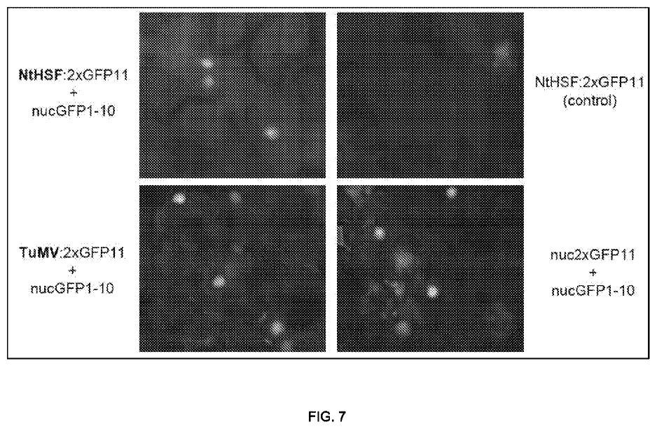

To do so, the present inventors targeted helper protein expression to the periplasm to facilitate inclusion of eukaryotic-like RNA into OMVs. Specifically, the present inventors followed the same experimental approach described above for the linear version of the constructs and were able to validate that introducing a hairpin-like structure to the RNA did not compromise ribosome binding and translation. Importantly, no GFP fluorescence will be observed in plant nuclei if the NLS:2×GFP11 peptide is not translated and the GFP11 protein properly targeted to the nuclei (FIGS. 6, 7). No GFP-positive nuclei were observed when UBQ:NLS:GFP1-10 was infiltrated into leaves for transient expression. When IRES:NLS:2×GFP11 constructs were transiently infiltrated into leaves with UBQ:NLS:GFP1-10 GFP positive signals were identified. Importantly, this observation indicates that the underlying design of the inventor's exemplary hairpin constructs are competent to drive eukaryotic translation and that the 5′ UTRs and the hairpin structure itself does not interfere with ribosome binding and correct translation. These data collectively demonstrate that all exemplary constructs proposed by the inventors produced positive GFP signals consistent with translation of Euk-mRNA in the eukaryotic cell.

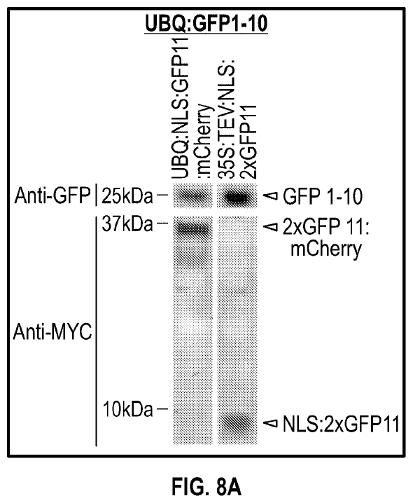

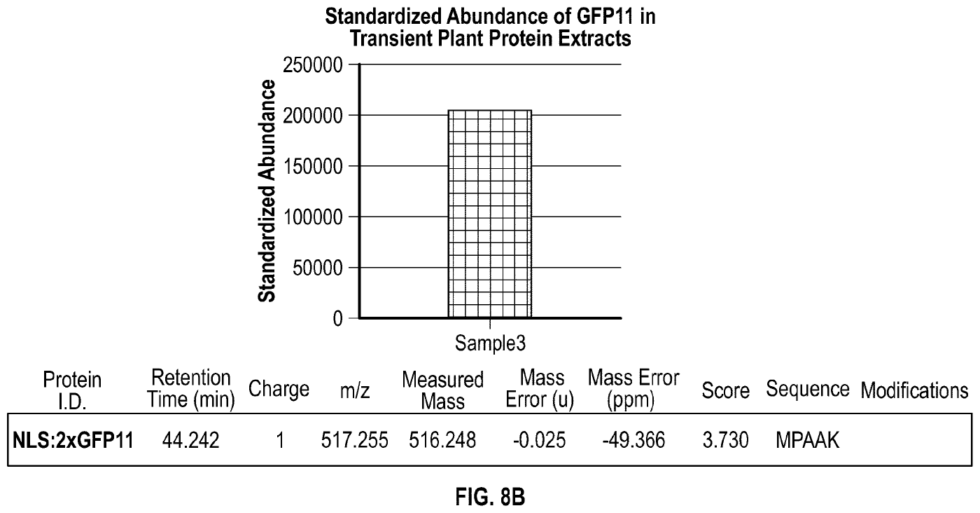

The present inventors further validated that bacterially produced and delivered Euk-mRNA mediated peptide synthesis is occurring in planta. Protein extracts were obtained from leaf discs of control plants (UBQ:NLS:GFP1-10+UBQ:NLS:GFP11:mCherry) and TEV:NLS:2×GFP11 co-infiltrated with UBQ:NLS:GFP1-10. As shown in FIG. 8, following total protein extraction and LC-MS/MS run we were able to identify unique peptides corresponding to NLS:2×GFP11 and NLS:GFP1-10. Together with the observed GFP positive nuclei both results underline that our combination of hairpin forming 5′ and 3′ UTR terminal sequences with IRES and CITE UTRs and poly-A tail is competent to drive eukaryotic translation of coding sequences. As a result, in a preferred embodiment, a eukaryotic-like RNA molecule transcribed by a prokaryotic organism may be introduced to host eukaryotic cell where it can be translated by eukaryotic ribosomes. In this preferred embodiment, delivery of one or more target mRNA molecules may be used to: express a target protein, generate a targeted phenotype in the host, delivery interfering RNA molecules, deliver gene editing transcripts for translation; and transform a eukaryotic cells or host without stable genetic integration.

### Example 6: Eukaryotic Translation in Eukaryotic Cells of Prokaryotic RNAs Expressed in Bacteria

Having established that an exemplary combination of regulatory (IRES, CITE, poly-A) and coding regions, which in this embodiment may include a signaling peptide and 2×GFP11 but that can be substituted by any other coding region or gene of interest) is competent to drive transcription of the peptide of interest (NLS:2×GFP11) the inventors generated the inventive Euk-mRNA expression system in prokaryotic cells. As described in the above section the inventors cloned eukaryotic-like RNA transcription units under a Ptac promoter of bacterial expression vector. Transcription of a subset of constructs was validated in both E. coli and Enterobacter cloacae genetic backgrounds (HT115 and E. cloacae Ae003 Δrnc (as described in Ser. No. 16/031,607, such description being incorporated herein by reference) (See FIG. 4) identified presence of the Euk-RNAs in outer membrane vesicles (OMVs) isolated from bacterial cultures (FIG. 4). Notably, OMVs are lipid vesicles released from the outer membrane of Gram-negative bacteria. OMVs may form an important means of communication among bacteria of the same species, as well as with other microorganisms in surrounding environment and importantly with its host. As such, the inventors sought to demonstrate that colonization of root tissue by bacteria expressing an exemplary eukaryotic like-mRNA construct would transfer of the coding RNA to root cells and its eventual translation in the eukaryotic cell.

To that end, to show that Euk-mRNAs could be produced by bacteria, delivered to the a eukaryote cell, in this case a plant cell, and successfully translated, the present inventors inoculated 2-4 day old Arabidopsis thaliana transgenic seedlings expressing GFP1-10 protein with bacteria expressing the various Euk-mRNA constructs. Bacterial inoculation was performed by co-incubating bacteria cultures expressing exemplary Euk-RNA nucleotide constructs with A. thaliana seedlings in Murashige-Skoog liquid media. Bacteria overnight starter cultures were used to inoculate fresh Luria-Bertani media with antibiotic selection and monitored to exponential growth phase (OD600=0.5-0.8). Bacteria were spun down and resuspended in MS media, seedlings were subsequently dipped in the bacteria suspension and incubated with agitation for 1 hour. A. thaliana Col-0 plants were used as negative control—Col-0 being the genetic background of the A. thaliana GFP1-10 transgenic plants. Plants were subsequently washed for 5 min in fresh MS buffer (×3) and plated on non-selective MS plates. Following 2 days to allow for bacterial colonization, plant GFP fluorescence was visualized 2-5 day post-inoculation.

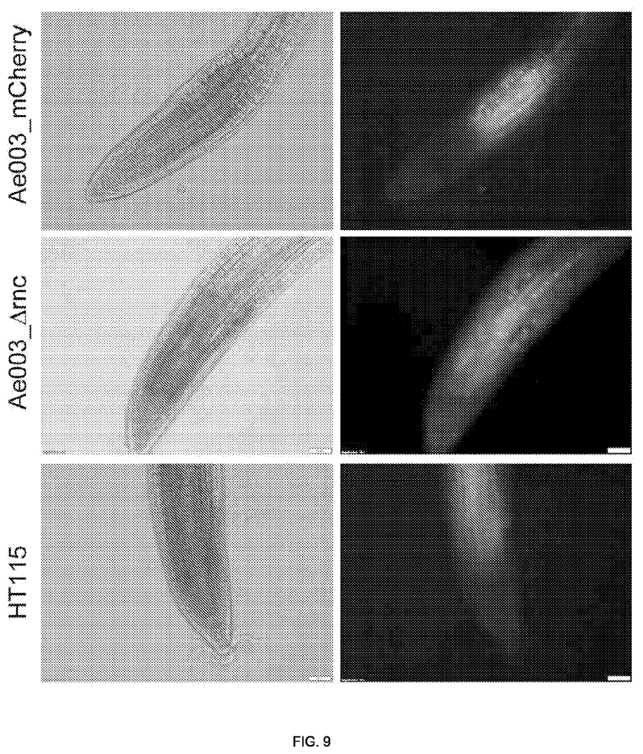

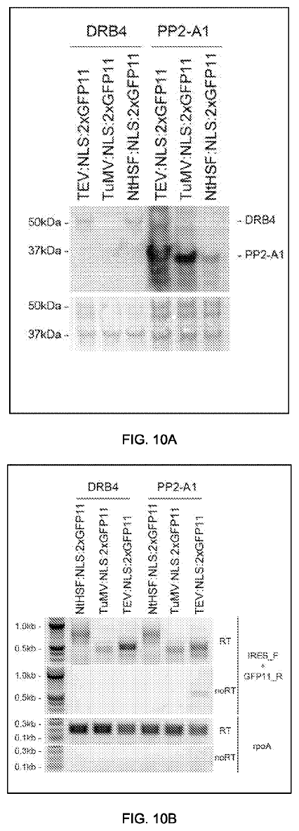

As shown in FIG. 9, roots of A. thaliana GFP1-10 transgenic plants displayed GFP positive nuclei in cells of the meristematic and elongation regions of the root tip. Positive GFP nuclei were observed in roots inoculated with E. coli HT115 Δrnc and E. cloacae Ae003 WT and Δrnc expressing the Euk-mRNA encoding GFP11. Importantly, no distinct GFP positive nuclei were observed in Col-0 plants. These data evidence trans-kingdom delivery of eukaryotic-like RNAs to eukaryotic plant cells where it is competent to recruit ribosomes and translated into NLS:2×GFP11 protein. These results further demonstrate inventive technology's ability to express a target RNA molecule in prokaryotic systems and export it to eukaryotic cells, where, in combination with ribosome recruiting sequence motifs (IRES/CITE) and poly-A tail it is competent to drive translation and give rise to a protein.

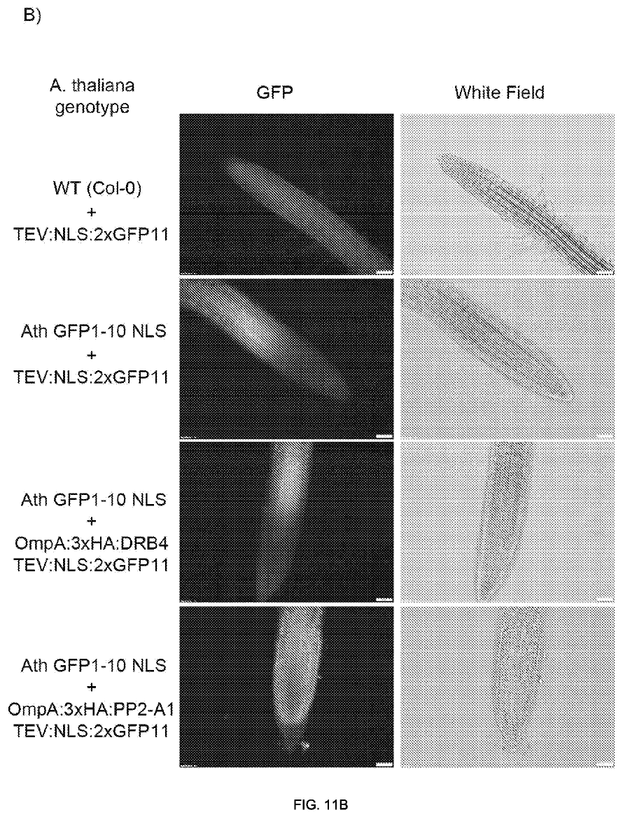

The inventors further validated that constructs with IRES NtHSF and crTMV and the CITE SNTV are also competent to drive translation in eukaryotic cells of an mRNA of interest—see Table 1 and FIG. 11.

### Example 7: Co-Expression of RNA Binding Helper Proteins Increase Trans-Kingdom Transport Efficiency of Euk-mRNAs from Donor Prokaryotes to Recipient Eukaryotic Cells

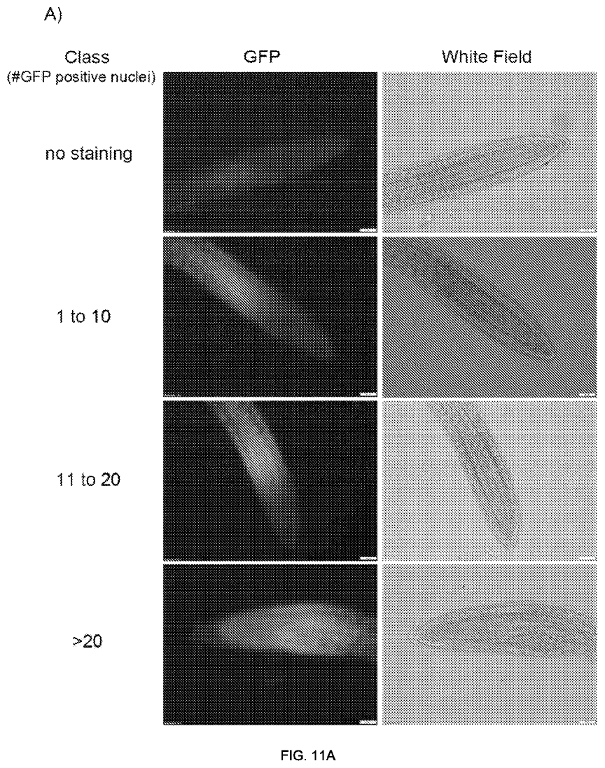

In addition, plasmid constructs encoding the IRES/CITE:NLS:2×GFP11 in combination with RNA binding helper proteins from A. thaliana, DRB4 (dsRNA binding protein 2) and PP2-A1 (phloem protein 2-A1), were tested by the inventors in root inoculation assays to determine if these proteins could act as chaperones for RNA trans-kingdom transfer. To this end, helper proteins were tagged with bacterial secretion peptides (e.g. OmpA, HylA) and cloned into the backbones encoding the various IREs/CITE:NLS:2×GFP11 genes, with transcription driven from its own promoter. As shown in FIG. 10, expression of hairpin eukaryotic-like RNAs in host E. coli HT115 cells was verified by RT-PCR as was helper protein production. These cultures were subsequently used to inoculate A. thaliana WT (Col-0) and A. thaliana NLS:GFP11 and GFP11:ER—endoplasmic reticulum 4-5 day old seedlings. Results are summarized in Table 1 and FIG. 11.

Three to four days post-inoculation root tips were examined in an epifluorescence microscope and the number of GFP positive nuclei scored to determine if the combination of Euk-mRNA with RNA binding helper proteins increased transport efficiency. For both TuMV:NLS:GFP11 and TEV:NLS:GFP11 hairpins in combination with expression of DRB4 and PP2-A1 resulted in an increase of GFP positive nuclei (Table 1) with >50% of roots (TEV) and >40% (TuMV) displaying +20 nuclei/root tip positive for GFP nuclei compared to plants inoculated with eukaryote mRNA alone. Statistical analysis of obtained data using the Kruskal-Wallis rank sum test highlighted significant differences between increased number of GFP positive nuclei in the presence of helper proteins DRB4 or PP2-A1 in TEV:NLS:2×GFP11 constructs relative to TEV:NLS:2×GFP11 alone (p=0.021). Same outcome was obtained when analyzing data for TuMV:NLS:2×GFP11 constructs with or without helper proteins with a p<0.001. Also, no GFP positive nuclei were observed in A. thaliana WT plants. Together these results indicate that the combination of RNA binding proteins, targeted for secretion with eukaryotic-like RNA improves transfer efficiency of the latter. We also validated that constructs with IRES NtHSF and crTMV and the CITE SNTV are also competent to drive translation in eukaryotic cells of an RNA of interest—see Table 1 for quantitation results. The obtained results support the application of this technology to genome editing approaches. Replacement of NLS:2×GFP11 coding sequence for that of a Cas3, Cas9 or a TALEN enzyme, for example, coding region (FIG. 11 showing an exemplary Cas3 or Cas9 construct which could be co-expressed with a guide RNA) would facilitate import of RNA to eukaryotic cells and translation of the editing machinery in the host cells in a non-heritable way.

### Example 8: Materials and Methods

Protein Digestion

Reduction, alkylation and trypsin digestion of proteins was performed following the University of Washington Proteomics Resource (UWPR). Briefly, protein concentration was measured using the A280 protein concentration assay on a NanoDrop 2000. Protein samples were then adjusted to pH ˜8 by dilution to a 0.1 M final concentration of ammonium bicarbonate (NH₄HCO₃) with a 1 M stock solution. Reduction of proteins was performed by the addition of a final concentration of 5 mM dithiothreitol (DTT) followed by incubation at approximately 56° C. for 30 minutes. After cooling, alkylation was performed in the dark at room temperature for 15 minutes by the addition of a final concentration of 14 mM iodoacetaminde (IAA) to the protein samples. Both DTT and IAA stocks were prepared at a concentration of 1 M in 0.1 M NH₄HCO₃. Lyophilized trypsin from bovine pancreas (Sigma) was solubilized in 1 mM CaCl₂ 50 mM Tris-HCl (pH=7.4) to a final concentration of 10 μg/μL and was added to proteins at a ratio of 1:50 trypsin:protein. Protein digests were incubated in a ThermoMixer C (Eppendorf) with 1.5 mL adapter maintained at 300 rpm at 37° C. overnight. Trypsin activity was quenched by addition of 1 M glacial acetic acid to a final concentration of 0.4% (pH=2).

Peptide Desalting

Peptides were then desalted and concentrated using ZipTip® C18 pipette tips (Sigma) following the UWPR ZipTip® Protocol. Briefly, ZipTip® pipette tips were pre-conditions 2 times with 10 μL 100% acetonitrile supplemented with 0.1% trifluoroacetic acid (TFA) followed by 4 times with 10 μL water with 0.1% TFA. Peptides were loaded by aspiration 5 times, and then washed 3 times with 10 μL water with 0.1% TFA. Peptides were then eluted into 100 μL 80% acetonitrile with 0.1% TFA, dried down in a CentriVap DNA Concentrator (Labconco) for 20 min under vacuum at 25° C., and then resolubilized in 3% acetonitrile with 0.1% formic acid supplemented with 50 fmol/μL of Glu¹-fibrinopeptide B (human; Sigma).

nanoLC-MS/MS

Peptides were analyzed by nanoLC-MS/MS using a Synapt G2-Si high definition mass spectrometer (HDMS) coupled to an Acquity M-Class ultra-performance liquid chromatograph (UPLC) with a nanoelectrospray ionization (nano-ESI) source (Waters). Peptides were separated using a trapping method, on a nanoEase M/Z Symmetry C18 Trap Column (180 μm I.D.×20 mm L; 5 μm particle size; Waters) followed by a nanoEase M/Z HSS C18 T3 Column (75 μm I.D.×150 mm L; 1.8 μm particle size; Waters) using mobile phases (A) water with 0.1% formic acid and (B) acetonitrile with 0.1% formic acid. 1 μL injections were made. Trapping was performed for 3 min at a flow rate of 10 μL/min with 1% B, followed by an analytical gradient performed for 75 min at a flow rate of 0.3 μL/min using the following linear gradient:

| Time | Flow rate | | |
|---|---|---|---|
| (min) | (0/min) | %A | %B |
| Initial | 0.3 | 99.0 | 1.0 |
| 0.1 | 0.3 | 99.0 | 1.0 |
| 32.1 | 0.3 | 60.0 | 40.0 |
| 35.1 | 0.3 | 60.0 | 40.0 |
| 45.1 | 0.3 | 20.0 | 80.0 |
| 50.1 | 0.3 | 20.0 | 80.0 |
| 55.1 | 0.3 | 99.0 | 1.0 |
| 75.0 | 0.3 | 99.0 | 1.0 |

Data-independent acquisition was performed from 5.0-60.0 minutes over a mass range of 300-2000 m/z in continuum mode. Polarity and ion optics were set to positive mode (ES+) and high-resolution mode, and capillary and cone voltages were set to 3.4 kV and 40 V, respectively. Source and desolvation temperatures were set to 100° C. and 200° C., nanoflow gas pressure was 0.5 Bar and desolvation gas flow was 650.0 L/hr. No collision energy was applied to low energy scans, and a high energy collision ramp of 12-40 V was applied to high energy scans. MS survey scan time was set to 0.5 s with a 0.014 s interscan delay. LockSpray measurements were acquired every 60 s using 100 fmol/μL Glu¹-fibrinopeptide B (785.8426 m/z) infused directly at 0.5 μL/min to maintain mass accuracy for MS measurements.

MS-Based Protein Identification

All peptide data were processed using Progenesis QI for proteomics (Nonlinear). Peptide spectra were searched against a custom database generated from our protein sequences from predicted translation of designed constructs as well as public databases (through Swissprot). The MSe identification workflow was used with default settings for Apex3D parameters, auto-alignment and peak picking. Alignment of peptide samples was conducted automatically using the most suitable fit. Autolysis peaks fitted to both trypsin and chymotrypsin autodigestion were observed; therefore, both trypsin and chymotrypsin digest reagents were used for peptide identification. In addition, non-specific cleavage was used to search for OmpA signaling peptide cleavage sites. Tolerance parameters and ion matching requirements for protein identification were set as follows: 25 ppm mass error for peptide ions, 2 fragments/peptide for peptide ion identification, 4% false discovery rate (FDR) and 2 peptides per protein for identification. Abundance of both non-conflicting and unique peptides was used for relative quantitation of identified proteins. Abundance values for identified proteins were exported into Excel as .csv files, and were standardized across samples using estimated protein concentration.

### REFERENCES

The following references are incorporated into the specification in their entirety:
[1] Kieft J (2008) Viral IRES RNA structures and ribosome interactions. Trends Biochem Sci.: 33(6): 274-283.
[2] Vanderhaeghen R, De Clercq R, Karimi M, Van Montagu M, Hilson P, Van Lijsebettens M (2006) Leader sequence of a plant ribosomal protein gene with complementarity to the 18S rRNA triggers in vitro cap-independent translation. FEBS letters 580: 2630-2636.
[3] Colussi T M, Costantino D A, Zhu J, Donohue J P, Korostelev A A, Jaafar Z A, Plank T D, Noller H F, Kieft J S (2015) Initiation of translation in bacteria by a structured eukaryotic IRES RNA. Nature 519:110-113.
[4] Nakashima N, Tamura T, Good L (2006) Paired termini stabilize antisense RNAs and enhance conditional gene silencing in Escherichia coli. Nuc. Acids Res. 34 (20): e138.
[5] Leppek K, Das R, Barna M (2018) Functional 5′ UTR mRNA structures in eukaryotic translation regulation and how to find them. Nature Reviews 19: 158-174.
[6] De Gregorio E, Preiss T, Hentze M W (1999) Translation driven by an eIF4G core domain in vivo. EMBO J. 18(17): 4865-4874.
[7] Song T, Mika F, Lindmark B, Liu Z, Schild S, Bishop A, Zhu J, Camilli A, Johansson J, Vogel J, Wai S N (2008) A new Vibrio cholerae sRNA modulates colonization and affects release of outer membrane vesicles. Mol Microbiol. 70(1): 100-111.
[8] Sanchez-Vargas I, Scott J C, Poole-Smith B K, Franz A W E, Barbosa-Solomieu V, Wilusz J, Olson K E, Blair C D (2009) Dengue Virus Type 2 Infections of Aedes aegypti Are Modulated by the Mosquito's RNA Interference Pathway. PLoS Pathogens 5(2): e1000299.
[9] Parker G, Eckert D M, Bass B L (2006) RDE-4 preferentially binds long dsRNA and its dimerization is necessary for cleavage of dsRNA to siRNA. RNA 12(5):807-818.
[10] Winston W M, Sutherlin M, Wright A J, Feinberg E H, Hunter C P (2007) Caenorhabditis elegans SID-2 is required for environmental RNA interference. PNAS 104(25):10565-10570.
[11] Bologna N G, Voinnet O (2014) The diversity, biogenesis, and activities of endogenous silencing small RNAs in Arabidopsis. Annu Rev. Plant Biol. 65:473-503.
[12] Park, W., Li, J., Song, R., Messing, J. & Chen, X. (2002) CARPEL FACTORY, a Dicer Homolog, and HEN1, a Novel Protein, Act in microRNA Metabolism in Arabidopsis thaliana. Curr. Biol. 12: 1484-1495.
[13] Xiao J, Feehery C E, Tzertzinis G, Maina C V (2009) E. coli RNase III(E38A) generates discrete-sized products from long dsRNA. RNA 15: 984-991.

### Tables

TABLE 1
Analysis of Arabidopsis thaliana roots for GFP positive nuclei. 3-5 day old Arabidopsis
thaliana seedlings of WT (Col-0) and transgenic lines expressing GFPI-10 tagged
with either signal (NLS) and endoplasmic reticulum (ER) were inoculated with
nuclear localization E. coli
HT115 transformed with eukaryotic-like RNA constructs
encoding GFP 11. Successful import by
of bacterially secreted GFP 11 coding RNAs and its subsequent
translation by plant cells the plant
cell's, by recruitment of ribosomes via IRES
and CITE interactions, into an NLS:2xGFP11 result in
reconstitution of splitGFP and fluorescence emission. NLS:2xGFP11 can
peptide will molecules of GFPI-10 and
traffic the reconstituted splitGFP to the nucleus. Scoring
bind two of nuclei will allow validation
of bacterial eukaryotic-like RNA transfer from
GFP positive bacteria and translation by plant
cells. See body of text for further result discussion. hpCHL1
is a non-coding hairpin RNA designed for RNAi
and is used as negative control in the context of this experiment. TEV,
TuMV, NtHSF, SNTV, CRTMV in table are used as abbreviations for
designation for IRES:NLS:2×GFP 11 coding constructs.

| Plasmid | A. thaliana line | 0 | 1 to 20 | 20+ |
|---|---|---|---|---|
| | WT | 5 | | |
| hpCHLI | GFP1-10:ER | 4 | 1 | |
| | NLS:GFP1-10 | 5 | | |
| | WT | 5 | | |
| PP2A1 hpCHLI | GFP1-10:ER | 4 | | 1 |
| | NLS:GFP1-10 | 4 | | 1 |
| | WT | 5 | | |
| TEV | GFP1-10:ER | 1 | 3 | 1 |
| | NLS:GFP1-10 | 4 | 1 | |
| | WT | 5 | | |
| DRB4 TEV | GFP1-10:ER | 1 | | 4 |
| | NLS:GFP1-10 | | 3 | 2 |
| | Col-0 | 5 | | |
| PP2A1 TEV | GFP1-10:ER | 2 | 1 | 2 |
| | NLS:GFP1-10 | 1 | | 4 |
| | WT | 5 | | |
| TuMV | GFP1-10:ER | 5 | | |
| | WT | 5 | | |
| DRB4 TuMV | GFP1-10:ER | 2 | | 3 |
| | NLS:GFP1-10 | 2 | 2 | 1 |
| | WT | 5 | | |
| PP2A1 TuMV | GFP1-10:ER | 4 | | 1 |
| | NLS:GFP1-10 | 1 | 1 | 3 |
| | WT | 5 | | |
| NtHSF | GFP1-10:ER | 3 | 2 | |
| | NLS:GFP1-10 | 5 | | |
| | WT | 5 | | |
| DRB4NtHSF | GFP1-10:ER | 4 | 1 | |
| | NLS:GFP1-10 | 5 | | |
| | WT | 5 | | |
| PP2A1 | GFP1-10:ER | 2 | 2 | 1 |
| CRTMV | | | | |
| | NLS:GFP1-10 | 4 | 1 | |
| | WT | 5 | | |
| PP2A1 SNTV | GFP1-10:ER | 5 | | |
| NLS:GFP1-10 | 3 | | 1 | |

#of GFP positive nuclei

A. thaliana Col-0 background

TABLE 2
primers used for RT-PCR analysis

| Primer Name | Sequence | |
|---|---|---|
| NtHSFl_IRES_F | GGCACGAGGCTCC | NtHSF1 specific F |
| | CATTAATATTTC | primer |
| TuMV_IRES_F | GGGAAAGCTTGCA | TuMV_specific F |
| | TGCCTG | primer to amplify |
| | | RNA construct, |
| | | without 5′ term |
| | | Duplex sequence |
| TEV_IRES_F | GAAATAACAAATC | TEV specific F |
| | TCAACACAACATA | primer to amplify |
| | TAC | RNA construct, |
| | | without 5′ term |
| | | Duplex sequence |
| rpoA-for | GCACCAAAGAAG | House-keeping gene |
| | GCGTTCAG | |
| rpoA-rev | GGTCAGGTG GCA | House-keeping gene |
| | GATCACAT | |

TABLE 3
Sequences of constructs and helper proteins:
Color legend where applicable:

Tac promoter (purple)
Paired-end termini duplex (yellow)
IRES/CITE Sequence (bolded)
Nuclear Localization signal (blue)
GGGS linker (brown)
GFP11 (green)
STOP (red)
Poly-A Tail (light purple)
Bacterial bi-directional terminator (light grey)

### Sequence Identification

As noted above, the instant application contains a full Sequence Listing which has been submitted electronically in ASCII format and is hereby incorporated by reference in its entirety. The following sequences are further provided herewith and are hereby incorporated into the specification in their entirety:

DNA
1-IRES:crTMV
Artificial
SEQ ID NO. 1
GAATTCGTCGATTCGGTTGCAGCATTTAAAGCGGTTGACAACTTTAAAAGAAGGAAAAAGAAGGTTGAAGAAAAGGG
TGTAGTAAGTAAGTATAAGTACAGACCGGAGAAGTACGCCGGTCCTGATTCGTTTAATTTGAAAGAAGAAAATGCCT
GCTGCGAAGAGGGTGAAATTGGATCCGGCGGCCAAACGAGTCAAACTTGATCCAGCTGCTAAGCGAGTGAAGCTAGA
CGGTGGTGGGGGGTCTGGGGGAGGTGGTAGCGGAGGTGGAGGTAGCAGAGACCACATGGTATTGCACGAATATGTAA
ATGCGGCTGGCATTACCGGAGGGGGGGGTAGCGGGGGCGGCGGATCCGGTGGTGGAAGCAGCAGAGACCATATGGTT
TTGCACGAGTATGTCAATGCCGCGGGCATAACGTAATAAAAAAAAAAAAAAAAAAAAAAAAAAAAAAAAAAAAAAAA
AAAAAAAAAAAACACCACCACCACCACCACTGCATGGTTAATTCCTCCTG
DNA
1-IRES:NtHSF
Artificial
SEQ ID NO. 2
GGCACGAGGCTCCCATTAATATTTCTTCTTCTGTGTAATTCCATTATTCTGTAGTAGATTCACGTCCGAGTTTAAAG
AAGAGAGAAAACTGAAAAGGCAGAAAATTCCAGAGCTTTAGATTTAGCCAAAGATAGTTATGGTCGTGTTGTTCTTG
GTGAAGATTGGCAAAGTAGGAGCCAATGGAAGAAACTAAGATCATAATCAATCGCCCCAAAAACAACCTTGTTCATT
CTATGGTTTTTCTCTTCGGTTTCTATGTTTGGGATTGGGAATTCCTCACTGTCCTTTTGCTTTTCAGTTATTGCTCC
TTCTAATTTTCCCTAGCTAGGATCTTCTCAATTAATTTCCTTTTTCATTTTCAACTAACTCATAATTAGCCCAAATC
TTCAAAAGAGTTTTGTGTAAGTTGATAGACGTTTAGAGAAACAGAGAAATACAGGGGAAAAACAAGGGATGCCTGCT
GCGAAGAGGGTGAAATTGGATCCGGCGGCCAAACGAGTCAAACTTGATCCAGCTGCTAAGCGAGTGAAGCTAGACGG
TGGTGGGGGGTCTGGGGGAGGTGGTAGCGGAGGTGGAGGTAGCAGAGACCACATGGTATTGCACGAATATGTAAATG
CGGCTGGCATTACCGGAGGGGGGGGTAGCGGGGGCGGCGGATCCGGTGGTGGAAGCAGCAGAGACCATATGGTTTTG
CACGAGTATGTCAATGCCGCGGGCATAACGTAATAAAAAAAAAAAAAAAAAAAAAAAAAAAAAAAAAAAAAAAAAAA
AAAAAAAAACACCACCACCACCACCACTGCATGGTTAATTCCTCCTGCAGATAAAAAAAATCCTTAGCTTTCGGTAA
GGATGATTTCT
DNA
1-IRES:TuMV
Artificial
SEQ ID NO. 3
GGGAAAGCTTGCATGCCTGCAGGTCGACTCTAGAAAAATATAAAAACTCAACACAACATACACAAAACGATTAAAGC
AAACACAATCTTTCAAAGCATTCAAAGCATTCAAGCAATCAAAGATTTTCAAATCTTTTGTCGTTATCAAAGCAATC
ACCAACAGGATCCAGGATCCCCGGGTGGTCAGTCCCTTATGCCTGCTGCGAAGAGGGTGAAATTGGATCCGGCGGCC
AAACGAGTCAAACTTGATCCAGCTGCTAAGCGAGTGAAGCTAGACGGTGGTGGGGGGTCTGGGGGAGGTGGTAGCGG
AGGTGGAGGTAGCAGAGACCACATGGTATTGCACGAATATGTAAATGCGGCTGGCATTACCGGAGGGGGGGGTAACG
GGGGCGGCGGATCCGGTGGTGGAAGCAGCAGAGACCATATGGTTTTGCACGAGTATGTCAATGCCGCGGOCATAACG
TAATAAAAAAAAAAAAAAAAAAAAAAAAAAAAAAAAAAAAAAAAAAAAAAAAAAAACACCACCACCACCACCACTCC
ATGGTTAATTCCTCCTGCAGATAAAAAAAATCCTTAGCTTTCGCTAAGGATGATTTCT
DNA
1-IRES:crTEV
Artificial
SEQ ID NO. 4
AAATAACAAATCTCAACACAACATATACAAAACAAACGAATCTCAAGCAATCAAGCATTCTACTTCTATTGCAGCAA
TTTAAATCATTTCTTTTAAAGCAAAAGCAATTTTCTGAAAATTTTCACCATTTACGAACGATAGCAATGCCTGCTGC
GAAGAGGGTGAAATTGGATCCGGCGGCCAAACGAGTCAAACTTGATCCAGCTGCTAAGCGAGTGAAGCTAGACGGTG
GTGGGGGGTCTGGGGGAGGTGGTAGCGGAGGTGGAGGTAGCAGAGACCACATGGTATTGCACGAATATGTAAATGCG
GCTGGCATTACCGGAGGGGGGGGTAGCGGGGGCGGCGGATCCGGTGGTGGAAGCAGCAGAGACCATATGGTTTTGCA
CGAGTATGTCAATGCCGCGGGCATAACGTAATAAAAAAAAAAAAAAAAAAAAAAAAAAAAAAAAAAAAAAAAAAAAA
AAAAAAACACCAGCACCACCACCACTGCATGGTTAATTCCTCCTGCAGATAAAAAAAATCCTTAGCTTTCGCTAAGG
ATGATTTCTGATATC
DNA
1-CITE:SNTV
Artificial
SEQ ID NO. 5
AAAAAAAAAAAAGTAAAGACAGGAAACTTTACTGACTAACATGCCTGCTGCGAAGAGGGTGAAATTGGATCCGGCGG
CCAAACGAGTCAAACTTGATCCAGCTGCTAAGCGAGTGAAGCTAGACGGTGGTGGGGGGTCTGGGGGAGGTGGTAGC
GGAGGTGGAGGTAGCAGAGACCACATGGTATTGCACGAATATGTAAATGCGGCTGGCATTACCGGAGGGGGGGGTAG
CGGGGGCGGCGGATCCGGTGGTGGAAGCAGCAGAGACCATATGGTTTTGCACGAGTATGTCAATGCCGCGGGCATAA
CGTAATAAAAAAAAAAAAAAAAAAAAAAAAAAAAAAAAAAAAAAAAAAAAAAAAAAAATCCCAGAGGTTCACAATGT
TAGTGATGGGGCGCTGAAAGATGCGTAGCTACCCTTCTGGAGCCACTTCCTGGTGGTAAGCAGAAATCCAAGGGTAC
GGTGGTACGGTGGAAAGCAGTCCCCACCACCACCACCACCACTGCATGGTTAATTCCTCCTG
DNA
2-IRES:crTMV (hairpin)
Artificial
SEQ ID NO. 6
CAGGAGGAATTAACCATGCAGTGGTGGTGGTGGTGGTGGAATTCGTGCATTCGGTTGCAGCATTTAAAGCGGTTGAC
AACTTTAAAAGAAGGAAAAAGAAGGTTGAAGAAAAGGGTGTAGTAAGTAAGTATAAGTACAGACCGGAGAAGTACGC
CGGTCCTGATTCGTTTAATTTGAAAGAAGAAAATGCCTGCTGCGAAGAGGGTGAAATTGGATCCGGCGGCCAAACGA
GTCAAACTTGATCCAGCTGCTAAGCGAGTGAAGCTAGACGGTGGTGGGGGGTCTGGGGGAGGTGGTAGCGGAGGTGG
AGGTAGCAGAGACCACATGGTATTGCACGAATATGTAAATGCGGCTGGCATTACCGGAGGGGGGGGTAGCGGGGGCG
GCGGATCCGGTGGTGGAAGCAGCAGAGACCATATGGTTTTGCACGAGTATGTCAATGCCGCGGGCATAACGTAATAA
AAAAAAAAAAAAAAAAAAAAAAAAAAAAAAAAAAAAAAAAAAAAAAAAAACACCACCACCACCACCACTGCATGGTT
AATTCCTCCTG
DNA
2-IRES:NtHSF (hairpin)
Artificial
SEQ ID NO. 7
CAGGAGGAATTAACCATGCAGTGGTGGTGGTGGTGGTGGGCACGAGGCTCCCATTAATATTTCTTCTTCTGTGTAAT
TCCATTATTCTGTAGTAGATTCACGTCCGAGTTTAAAGAAGAGAGAAAACTGAAAAGGCAGAAAATTCCAGAGCTTT
AGATTTAGCCAAAGATAGTTATGGTCGTGTTGTTCTTGGTGAAGATTGGCAAAGTAGGAGCCAATGGAAGAAACTAA
GATCATAATCAATCGCCCCAAAAACAACCTTGTTCATTCTATGGTTTTTCTCTTCGGTTTCTATGTTTGGGATTGGG
AATTCCTCACTGTCCTTTTGCTTTTCAGTTATTGCTCCTTCTAATTTTCCCTAGCTAGGATCTTCTCAATTAATTTC
CTTTTTCATTTTCAACTAACTCATAATTAGCCCAAATCTTCAAAAGAGTTTTGTGTAAGTTGATAGACGTTTAGAGA
AACAGAGAAATACAGGGGAAAAACAAGGGATGCCTGCTGCGAAGAGGGTGAAATTGGATCCGGCGGCCAAACGAGTC
AAACTTGATCCAGCTGCTAAGCGAGTGAAGCTAGACGGTGGTGGGGGGTCTGGGGGAGGTGGTAGCGGAGGTGGAGG
TAGCAGAGACCACATGGTATTGCACGAATATGTAAATGCGGCTGGCATTACCGGAGGGGGGGGTAGCGGGGGCGGCG
GATCCGGTGGTGGAAGCAGCAGAGACCATATGGTTTTGCACGAGTATGTCAATGCCGCGGGCATAACGTAATAAAAA
AAAAAAAAAAAAAAAAAAAAAAAAAAAAAAAAAAAAAAAAAAAAAAACACCACCACCACCACCACTGCATGGTTAAT
TCCTCCTGCAGATAAAAAAAATCCTTAGCTTTCGCTAAGGATGATTTCT
DNA
2-IRES:crTEV(hairpin)
Artificial
SEQ ID NO. 8
CAGGAGGAATTAACCATGCAGTGGTGGTGGTGGTGGTGAAATAACAAATCTCAACACAACATATACAAAACAAACGA
ATCTCAAGCAATCAAGCATTCTACTTCTATTGCAGCAATTTAAATCATTTCTTTTAAAGCAAAAGCAATTTTCTGAA
AATTTTGACCATTTACGAACGATAGCAATGCCTGCTGCGAAGAGGGTGAAATTGGATCCGGCGGCCAAACGAGTCAA
ACTTGATCCAGCTGCTAAGCGAGTGAAGCTAGACGGTGGTGGGGGGTCTGGGGGAGGTGGTAGCGGAGGTGGAGGTA
GCAGAGACCACATGGTATTGCACGAATATGTAAATGCGGCTGGCATTACCGGAGGGGGGGGTAGCGGGGGCGGCGGA
TCCGGTGGTGGAAGCAGCAGAGACCATATGGTTTTGCACGAGTATGTCAATGCCGCGGGCATAACGTAATAAAAAAA
AAAAAAAAAAAAAAAAAAAAAAAAAAAAAAAAAAAAAAAAAAAACACCACCACCACCACCACTTGCATGGTTAATTC
CTCCTGCAGATAAAAAAAATCCTTAGCTTTCGCTAAGGATGATTTCTGATATC
DNA
2-IRES:TuMV (hairpin)
Artificial
SEQ ID NO. 9
CAGGAGGAATTAACCATGCAGTGGTGGTGGTGGTGGTGGGGAAAGCTTGCATGCCTGCAGGTCGACTCTAGAAAAAT
ATAAAAACTCAACACAACATACACAAAACGATTAAAGCAAACACAATCTTTCAAAGCATTCAAAGCATTCAAGCAAT
CAAAGATTTTCAAATCTTTTGTCGTTATCAAAGCAATCACCAACAGGATCCAGGATCCCCGGGTGGTCAGTCCCTTA
TGCCTGCTGCGAAGAGGGTGAAATTGGATCCGGCGGCCAAACGAGTCAAACTTGATCCAGCTGCTAAGCGAGTGAAG
CTAGACGGTGGTGGGGGGTCTGGGGGAGGTGGTAGCGGAGGTGGAGGTAGCAGAGACCACATGGTATTGCACGAATA
TGTAAATGCGGCTGGCATTACCGGAGGGGGGGGTAGCGGGGGCGGCGGATCCGGTGGTGGAAGCAGCAGAGACCATA
TGGTTTTGCACCAGTATGTCAATGCCGCGGGCATAACGTAATAAAAAAAAAAAAAAAAAAAAAAAAAAAAAAAAAAA
AAAAAAAAAAAAAAAAACACCACCACCACCACCACTGCATGGTTAATTCCTCCTGCAGATAAAAAAAATCCTTAGCT
TTCGCTAAGGATGATTTCT
DNA
2-CITE:SNTV (hairpin)
Artificial
SEQ ID NO. 10
CAGGAGGAATTAACCATGCAGTGGTGGTGGTGGTGGTGAAAAAAAAAAAAGTAAAGACAGGAAACTTTACTGACTAA
CATGCCTGCTGCGAAGAGGGTGAAATTGGATCCGGCGGCCAAACGAGTCAAACTTGATCCAGCTGCTAAGCGAGTGA
AGCTACACGGTGGTGGGGGGTCTGGGGGAGGTGGTAGCGGAGGTGGAGGTAGCAGAGACCACATGGTATTGCACGAA
TATGTAAATGCGGCTGGCATTACCGGAGGGGGGGGTAGCGGGGGCGGCGGATCCGGTGGTGGAAGCAGCAGAGACCA
TATGGTTTTGCACGAGTATGTCAATGCCGCGGGCATAACGTAATAAAAAAAAAAAAAAAAAAAAAAAAAAAAAAAAA
AAAAAAAAAAAAAAAAAAATCCCAGAGGTTCACAATGTTAGTGATGGGGCGCTGAAAGATGCGTAGCTACCCTTCTG
GAGCCACTTCCTGGTGGTAAGCAGAAATCCAAGGGTACGGTGGTACGGTGGAAAGCAGTCCCCACCACCACCACCAC
CACTGCATGGTTAATTCCTCCTG
DNA
ompA secretion signal
E. Coli
SEQ ID NO. 10
ATGAAAAAGACGGCGATTGCTATCGCTGTGGCGCTTGCTGGATTCGCCACTGTAGCACAAGCA
Amino Acid
ompA secretion signal
E. Coli
SEQ ID NO. 11
MKKTAIAIAVALAGFATVAQA
Amino Acid
PelB (pectate lyase B)
Erwinia carotovora
SEQ ID NO. 12
MKYLLPTAAAGLLLLAAQPAMA
Amino Acid
StII (heat-stable enterotoxin 2)
bacteria
SEQ ID NO. 13
MKKNIAFLLASMFVFSIATNAYA
Amino Acid
Endoxylanase
Bacillus sp.
SEQ ID NO. 14
MFKFKKKFLVGLTAAFMSISMFSATASA
Amino Acid
PhoA (alkaline phosphatase)
E. Coli
SEQ ID NO. 15
MKQSTIALALLPLLFTPVTKA
Amino Acid
OmpF (outer-membrane protein F)
E. Coli
SEQ ID NO. 16
MMKRNILAVIVPALLVAGTANA
Amino Acid
PhoE (outer-membrane pore protein E)
E. Coli
SEQ ID NO. 17
MKKSTLALVVMGIVASASVQA
Amino Acid
MalE (maltose-binding protein)
E. Coli
SEQ ID NO. 18
MKIKTGARILALSALTTMMFSASALA
Amino Acid
OmpC (outer-membrane protein C)
E. Coli
SEQ ID NO. 19
MKVKVLSLLVPALLVAGAANA
Amino Acid
Lpp (murein lipoprotein)
E. Coli
SEQ ID NO. 20
MKATKLVLGAVILGSTLLAG
Amino Acid
LamB (λ, receptor protein)
E. Coli
SEQ ID NO. 21
MMITLRKLPLAVAVAAGVMSAQAMA
Amino Acid
OmpT (protease VII)
E. Coli
SEQ ID NO. 22
MRAKLLGIVLTTPIAISSFA
Amino Acid
HylA (a-Haemolysin)
E. Coli
SEQ ID NO. 23
SKQHYGIRKYKVGVCSALIALSILG
DNA
dsRNA binding protein 2 (DRB4)
Arabidopsis thaliana
SEQ ID NO. 24
GACCACGTGTACAAGGGTCAGCTGCAAGCGTATGCGCTGCAGCACAACCTGGAGCTGCCGGTTTACGCGAACGAGCG
TGAAGGTCCGCCGCACGCGCCGCGTTTCCGTTGCAACGTGACCTTCTGCGGCCAGACCTTTCAAAGCAGCGAGTTCT
TTCCGACCCTGAAAAGCGCGGAACACGCGGCGGCGAAGATCGCGGTGGCGAGCCTGACCCCGCAAAGCCCGGAAGGT
ATCGATGTTGCGTACAAAAACCTGCTGCAGGAGATTGCGCAAAAGGAAAGCAGCCTGCTGCCGTTCTATGCGACCGC
GACCAGCGGTCCGAGCCACGCGCCGACCTTTACCAGCACCGTGGAGTTCGCGGGTAAAGTTTTTAGCGGCGAGGAAG
CGAAGACCAAGAAACTGGCGGAAATGAGCGCGGCGAAAGTTGCGTTCATGAGCATTAAGAACGGTAACAGCAACCAG
ACCGGTAGCCCGACCCTGCCGAGCGAGCGTCAAGAAGACGTGAACAGCAACGTTAAAAGCAGCCCGCAGGAGATCCA
CAGCCAACCGAGCAGCAAGGTGGTTATGACCCCGGACACCCCGAGCAAAGGTATTAAGGTTAACGAGGATGAATTTC
CGGACCTGCACGATGCGCCGGCGAGCAACGCGAAAGAAATCAACGTGGCGCTGAACGAGCCGGAAAACCCGACCAAC
GACGGTACCCTGAGCGCGCTGACCACCGATGGCATGAAGATGAACATCGCGAGCAGCAGCCTGCCGATTCCGCACAA
CCCGACCAACGTTATTACCCTGAACGCGCCGGCGGCGAACGGTATCAAGCGTAACATTGCGGCGTGCAGCAGCTGGA
TGCCGCAGAACCCGACCAACGACGGCAGCGAGACCAGCAGCTGCGTGGTTGATGAGAGCGAAAAGAAAAAGCTGATC
ATGGGTACCGGTCACCTGAGCATTCCGACCGGTCAGCACGTGGTTTGCCGTCCGTGGAACCCGGAGATCACCCTGCC
GCAAGATGCGGAAATGCTGTTCCGTGACGATAAATTTATTGCGTATCGTCTGGTGAAGCCGTAA
Amino Acid
dsRNA binding protein 2 (DRB4)
Arabidopsis thaliana
SEQ ID NO. 25
MDHVYKGQLQAYALQHNLELPVYANEREGPPHAPRFRCNVTFCGQTFQSSEFFPTLKSAEHAAAKIAVASLTPQSPE
GIDVAYKNLLQEIAQKESSLLPFYATATSGPSHAPTFTSTVEFAGKVFSGEEAKTKKLAEMSAAKVAFMSIKNGNSN
QTGSPTLPSERQEDVNSNVKSSPQEIHSQPSSKVVMTPDTPSKGIKVNEDEFPDLHDAPASNAKEINVALNEPENPT
NDGTLSALTTDGMKMNIASSSLPIPHNPTNVITLNAPAANGIKRNIAACSSWMPQNPTNDGSETSSCVVDESEKKKL
IMGTGHLSIPTGQHVVCRPWNPEITLPQDAEMLFRDDKFIAYRLVKP
DNA
phloem protein 2-A1 (PP2-A1)
Arabidopsis thaliana
SEQ ID NO. 26
AAGCAAGAAACACTGCAGCGAGCTGCTGCCGAACAAGATGTTCCGTAACCAGGACAGCAAGTACCTGATCCCGGTTC
AAAAAGAAGCGCCGCCGGTGACCACCCTGCCGATGAAGGCGAGCACCGTTAAAAGCCCGCACAACTGCGAGGCGATC
CTGCGTGACGCGGATCCGCCGATTAGCCTGAGCAGCGTTAACCTGAGCGAACAGCTGCGTAGCGGCGTGTTCCTGAA
GCCGAAGAAACAAATCAAATACTGGGTTGATGAGCGTAACAGCAACTGCTTCATGCTGTTTGCGAAGAACCTGAGCA
TTACCTGGAGCGACGATGTGAACTATTGGACCTGGTTTACCGAGAAAGAAAGCCCGAACGAGAACGTTGAAGCGGTG
GGTCTGAAGAACGTGTGCTGGCTGGACATCACCGGCAAATTCGATACCCGTAACCTGACCCCGGGTATTGTTTACGA
GGTGGTTTTTAAGGTGAAACTGGAAGACCCGGCGTATGGCTGGGATACCCCGGTTAACCTGAAACTGGTGCTGCCGA
ACGGCAAGGAGAAACCGCAGGAAAAGAAAGTTAGCCTGCGTGAACTGCCGCGTTACAAGTGGGTGGACGTTCGTGTG
GGCGAGTTCGTGCCGGAGAAGAGCGCGGCGGGCGAGATCACCTTTAGCATGTATGAACACGCGGCGGGTGTTTGGAA
GAAAGGCCTGAGCCTGAAGGGTGTGGCGATTCGTCCGAAACAATAA
Amino Acid
phloem protein 2-Al (PP2-A1)
Arabidopsis thaliana
SEQ ID NO. 27
MSKKHCSELLPNKMFRNQDSKYLIPVQKEAPPVTTLPMKASTVKSPHNCEAILRDADPPISLSSVNLSEQLRSGVFL
KPKKQIKYWVDERNSNCFMLFAKNLSITWSDDVNYWTWFTEKESPNENVEAVGLKNVCWLDITGKFDTRNLTPGIVY
EVVFKVKLEDPAYGWDTPVNLKLVLPNGKEKPQEKKVSLRELPRYKWVDVRVGEFVPEKSAAGEITFSMYEHAAGVW
KKGLSLKGVAI RPKQ
DNA
Kozak (Shine Delgarno) Sequence
Artificial
SEQ ID NO. 28
AGGAGGTACCCACC
DNA
Cas3
Streptococcus thermophilus
SEQ ID NO. 29
ATGAAACATATTAATGATTATTTTTGGGCTAAGAAAACAGAGGAAAATAGTAGACTTCTTTGGTTACCATTAACTCAACA
CTTAGAAGACACGAAAAATATTGCAGGCCTCTTATGGGAACATTGGTTAAGTGAAGGACAAAAGGTATTAATTGAAAATT
CTATTAATGTTAAATCAAATATTGAAAACCAAGGGAAAAGATTGGCACAATTCCTAGGAGCTGTTCATGATATCGGTAAA
GCAACACCAGCTTTTCAGACGCAAAAAGGTTATGCAAATTCAGTAGATTTGGATATTCAATTGTTAGAAAAATTGGAACG
CGCAGGTTTTTCTGGCATTAGTTCTCTCCAACTAGCCTCCCCCAAAAAGAGTCATCATAGCATTGCAGGTCAATATTTGT
TATCCCATTATGGCGTGGACGAAGATATTGCAACAATTATTGGTGGACACCATGGACGACCAGTTGATGATTTAGACGGT
TTAAATTCTCAAAAAAGCTATCCCTCCAATTATTACCAGGATGAAAAGAAAGATAGTCTCGTTTATCAGAAATGGAAGTC
AAATCAAGAAGCTTTTTTAAACTGGGCTTTAACAGAAACAGGGTTTAATTCTGTGTCTCAGCTTCCAAAAATCAAACAGC
CTGCTCAAGTTATTCTATCAGGTTTACTCATAATGTCTGACTGGATTGCTAGTAATGAGCATTTTTTTCCTTTGTTAAGT
TTGGATGAAACTGATGTGAAAAACAAGAGTCAACGTATTGAAACTGGGTTTAAAAAGTGGAAAAAATCTAACTTGTGGCA
ACCTGAAACTTTCGTTGACCTTGTTACTCTTTATCAGGAAAGATTTGGATTTAGTCCACGAAATTTTCAGCTGATACTCT
CACAAACAATCGAAAAGACGACTAATCCTGGGATAGTGATACTGGAAGCGCCAATGGGAATCGGGAAAACAGAGGCGGCT
CTAGCGGTATCAGAGCAGTTATCTAGTAAAAAAGGATGTAGTGGATTGTTTTTTGGATTGCCCACACAAGCAACCTCCAA
TGGAATTTTTAAGAGGATTGAACAGTGGACAGAGAATATAAAGGGTAACAATTCTGATCATTTTTCCATTCAGCTGGTTC
ATGGAAAAGCAGCCTTAAATACGGATTTTATTGAGTTACTTAAAGGAAATACAATTAATATGGACGACTCGGAAAACGGC
AGTATTTTTGTCAATGAGTGGTTTTCTGGGAGAAAAACTTCAGCATTAGATGATTTTGTAGTTGGGACGGTCGACCAATT
TTTAATGGTGGCTTTAAAACAAAAACATTTGGCCTTACGTCATTTAGGATTTAGTAAAAAAGTTATCGTTATTGATGAAG
TCCACGCTTATGATGCTTATATGAGCCAATATTTGTTGGAAGCTATCAGATGGATGGGAGCTTATGGTGTTCCTGTAATT
ATTTTATCAGCAACTTTACCTGCCCAACAAAGAGAAAAACTCATAAAAAGCTATATGGCTGGAATGGGAGTGAAATGGCG
AGATATTGAAAATATAGATCAGATAAAAATAGACGCATACCCTTTAATCACTTATAATGACGGGCCTGACATTCATCAAG
TTAAAATGTTCGAAAAGCAAGAACAAAAAAATATCTACATTCATCGTTTACCAGAAGAACAGTTATTTGATATTGTAAAA
GAAGGTCTTGACAATGGTGGAGTAGTTGGGATAATTGTCAATACGGTGAGAAAATCTCAAGAATTGGCAAGAAATTTTTC
AGATATTTTTGGAGATGATATGGTAGATTTGCTTCATTCTAATTTCATAGCAACTGAAAGAATCCGAAAAGAAAAGGATT
TATTGCAAGAAATTGGGAAAAAAGCAATACGTCCACCAAAGAAAATCATTATTGGTACACAGGTGCTTGAACAGTCGTTA
GATATTGATTTTGATGTACTGATAAGCGACTTAGCGCCTATGGATTTACTCATTCAACGTATCGGACGACTACATCGTCA
CAAAATCAAAAGGCCCCAAAAGCACGAAGTAGCAAGATTTTATGTTTTAGGAACATTTGAAGAGTTTGATTTTGATGAAG
GAACGCGTTTGGTTTATGGGGACTACCTATTAGCTAGAACTCAGTACTTTTTACCAGATAAAATACGACTTCCTGATGAT
ATTTCACCGCTAGTCCAAAAGGTTTATAATTCAGACCTAACAATTACGTTTCCAAAGCCAGAACTTCATAAAAAATATTT
GGATGCTAAAATAGAACATGATGATAAGATTAAAAATAAAGAAACAAAGGCAAAGTCATACCGTATTGCTAATCCTGTCT
TAAAAAAATCGAGAGTTCGAACTAACAGTTTGATTGGTTGGTTAAAGAACCTCCATCCAAATGATAGTGAAGAAAAAGCA
TATGCTCAAGTTCGAGATATTGAAGATACAGTTGAAGTGATTGCATTAAAAAAAATATCTGATGGGTATGGTTTGTTCAT
AGAAAATAAAGATATATCTCAGAACATTACTGATCCTATAATTGCAAAAAAGGTAGCACAAAATACTTTACGACTTCCGA
TGAGTTTATCCAAAGCCTATAATATTGATCAAACGATTAATGAGCTTGAAAGATATAACAATAGCCACTTAAGTCAATGG
CAAAACTCATCATGGTTAAAGGGATCTCTTGGGATTATTTTTGATAAAAACAATGAGTTTATACTGAATGGATTTAAACT
ATTATATGATGAAAAATATGGTGTTACCATAGAAAGGTTGGATAAGAATGAGTCGGTTTAA
Amino Acid
Cas3
Streptococcus thermophiles
SEQ ID NO. 30
MKHINDYFWAKKTEENSRLLWLPLTQHLEDTKNIAGLLWEHWLSEGQKVLIENSINVKSNIENQGKRLAQFLGAVHDIGK
ATPAFQTQKGYANSVDLDIQLLEKLERAGFSGISSLQLASPKKSHHSIAGQYLLSHYGVDEDIATIIGGHHGRPVDDLDG
LNSQKSYPSNYYQDEKKDSLVYQKWKSNQEAFLNWALTETGFNSVSQLPKIKQPAQVILSGLLIMSDWIASNEHFFPLLS
LDETDVKNKSQRIETGFKKWKKSNLWQPETFVDLVTLYQERFGFSPRNFQLILSQTIEKTTNPGIVILEAPMGIGKTEAA
LAVSEQLSSKKGCSGLFFGLPTQATSNGIFKRIEQWTENIKGNNSDHFSIQLVHGKAALNTDFIELLKGNTINMDDSENG
SIFVNEWFSGRKTSALDDFVVGTVDQFLMVALKQKHLALRHLGFSKKVIVIDEVHAYDAYMSQYLLEAIRWMGAYGVPVI
ILSATLPAQQREKLIKSYMAGMGVKWRDIENIDQIKIDAYPLITYNDGPDIHQVKMFEKQEQKNIYIHRLPEEQLFDIVK
EGLDNGGVVGIIVNTVRKSQELARNFSDIFGDDMVDLLHSNFIATERIRKEKDLLQEIGKKAIRPPKKIIIGTQVLEQSL
DIDFDVLISDLAPMDLLIQRIGRLHRHKIKRPQKHEVARFYVLGTFEEFDFDEGTRLVYGDYLLARTQYFLPDKIRLPDD
ISPLVQKVYNSDLTITFPKPELHKKYLDAKIEHDDKIKNKETKAKSYRIANPVLKKSRVRTNSLIGWLKNLHPNDSEEKA
YAQVRDIEDTVEVIALKKISDGYGLFIENKDISQNITDPIIAKKVAQNTLRLPMSLSKAYNIDQTINELERYNNSHLSQW
QNSSWLKGSLGIIFDKNNEFILNGFKLLYDEKYGVTIERLDKNESV
DNA
Cas9
Streptococcus
SEQ ID NO. 31
ATGGATAAGAAATACTCAATAGGCTTAGATATCGGCACAAATAGCGTCGGATGGGCGGTGATCACTGATGAATATAA
GGTTCCGTCTAAAAAGTTCAAGGTTCTGGGAAATACAGACCGCCACAGTATCAAAAAAAATCTTATAGGGGCTCTTT
TATTTGACAGTGGAGAGACAGCGGAAGCGACTCGTCTCAAACGGACAGCTCGTAGAAGGTATACACGTCGGAAGAAT
CGTATTTGTTATCTACAGGAGATTTTTTCAAATGAGATGGCGAAAGTAGATGATAGTTTCTTTCATCGACTTGAAGA
GTCTTTTTTGGTGGAAGAAGACAAGAAGCATGAACGTCATCCTATTTTTGGAAATATAGTAGATGAAGTTGCTTATC
ATGAGAAATATCCAACTATCTATCATCTGCGAAAAAAATTGGTAGATTCTACTGATAAAGCGGATTTGCGCTTAATC
TATTTGGCCTTAGCGCATATGATTAAGTTTCGTGGTCATTTTTTGATTGAGGGAGATTTAAATCCTGATAATAGTGA
TGTGGACAAACTATTTATCCAGTTGGTACAAACCTACAATCAATTATTTGAAGAAAACCCTATTAACGCAAGTGGAG
TAGATGCTAAAGCGATTCTTTCTGCACGATTGAGTAAATCAAGACGATTAGAAAATCTCATTGCTCAGCTCCCCGGT
GAGAAGAAAAATGGCTTATTTGGGAATCTCATTGCTTTGTCATTGGGTTTGACCCCTAATTTTAAATCAAATTTTGA
TTTGGCAGAAGATGCTAAATTACAGCTTTCAAAAGATACTTACGATGATGATTTAGATAATTTATTGGCGCAAATTG
GAGATCAATATGCTGATTTGTTTTTGGCAGCTAAGAATTTATCAGATGCTATTTTACTTTCAGATATCCTAAGAGTA
AATACTGAAATAACTAAGGCTCCCCTATCAGCTTCAATGATTAAACGCTACGATGAACATCATCAAGACTTGACTCT
TTTAAAAGCTTTAGTTCGACAACAACTTCCAGAAAAGTATAAAGAAATCTTTTTTGATCAATCAAAAAACGGATATG
CAGGTTATATTGATGGGGGAGCTAGCCAAGAAGAATTTTATAAATTTATCAAACCAATTTTAGAAAAAATGGATGGT
ACTGAGGAATTATTGGTGAAACTAAATCGTGAAGATTTGCTGCGCAAGCAACGGACCTTTGACAACGGCTCTATTCC
CCATCAAATTCACTTGGGTGAGCTGCATGCTATTTTGAGAAGACAAGAAGACTTTTATCCATTTTTAAAAGACAATC
GTGAGAAGATTGAAAAAATCTTGACTTTTCGAATTCCTTATTATGTTGGTCCATTGGCGCGTGGCAATAGTCGTTTT
GCATGGATGACTCGGAAGTCTGAAGAAACAATTACCCCATGGAATTTTGAAGAAGTTGTCGATAAAGGTGCTTCAGC
TCAATCATTTATTGAACGCATGACAAACTTTGATAAAAATCTTCCAAATGAAAAAGTACTACCAAAACATAGTTTGC
TTTATGAGTATTTTACGGTTTATAACGAATTGACAAAGGTCAAATATGTTACTGAAGGAATGCGAAAACCAGCATTT
CTTTCAGGTGAACAGAAGAAAGCCATTGTTGATTTACTCTTCAAAACAAATCGAAAAGTAACCGTTAAGCAATTAAA
AGAAGATTATTTCAAAAAAATAGAATGTTTTGATAGTGTTGAAATTTCAGGAGTTGAAGATAGATTTAATGCTTCAT
TAGGTACCTACCATGATTTGCTAAAAATTATTAAAGATAAAGATTTTTTGGATAATGAAGAAAATGAAGATATCTTA
GAGGATATTGTTTTAACATTGACCTTATTTGAAGATAGGGAGATGATTGAGGAAAGACTTAAAACATATGCTCACCT
CTTTGATGATAAGGTGATGAAACAGCTTAAACGTCGCCGTTATACTGGTTGGGGACGTTTGTCTCGAAAATTGATTA
ATGGTATTAGGGATAAGCAATCTGGCAAAACAATATTAGATTTTTTGAAATCAGATGGTTTTGCCAATCGCAATTTT
ATGCAGCTGATCCATGATGATAGTTTGACATTTAAAGAAGACATTCAAAAAGCACAAGTGTCTGGACAAGGCGATAG
TTTACATGAACATATTGCAAATTTAGCTGGTAGCCCTGCTATTAAAAAAGGTATTTTACAGACTGTAAAAGTTGTTG
ATGAATTGGTCAAAGTAATGGGGCGGCATAAGCCAGAAAATATCGTTATTGAAATGGCACGTGAAAATCAGACAACT
CAAAAGGGCCAGAAAAATTCGCGAGAGCGTATGAAACGAATCGAAGAAGGTATCAAAGAATTAGGAAGTCAGATTCT
TAAAGAGCATCCTGTTGAAAATACTCAATTGCAAAATGAAAAGCTCTATCTCTATTATCTCCAAAATGGAAGAGACA
TGTATGTGGACCAAGAATTAGATATTAATCGTTTAAGTGATTATGATGTCGATCACATTGTTCCACAAAGTTTCCTT
AAAGACGATTCAATAGACAATAAGGTCTTAACGCGTTCTGATAAAAATCGTGGTAAATCGGATAACGTTCCAAGTGA
AGAAGTAGTCAAAAAGATGAAAAACTATTGGAGACAACTTCTAAACGCCAAGTTAATCACTCAACGTAAGTTTGATA
ATTTAACGAAAGCTGAACGTGGAGGTTTGAGTGAACTTGATAAAGCTGGTTTTATCAAACGCCAATTGGTTGAAACT
CGCCAAATCACTAAGCATGTGGCACAAATTTTGGATAGTCGCATGAATACTAAATACGATGAAAATGATAAACTTAT
TCGAGAGGTTAAAGTGATTACCTTAAAATCTAAATTAGTTTCTGACTTCCGAAAAGATTTCCAATTCTATAAAGTAC
GTGAGATTAACAATTACCATCATGCCCATGATGCGTATCTAAATGCCGTCGTTGGAACTGCTTTGATTAAGAAATAT
CCAAAACTTGAATCGGAGTTTGTCTATGGTGATTATAAAGTTTATGATGTTCGTAAAATGATTGCTAAGTCTGAGCA
AGAAATAGGCAAAGCAACCGCAAAATATTTCTTTTACTCTAATATCATGAACTTCTTCAAAACAGAAATTACACTTG
CAAATGGAGAGATTCGCAAACGCCCTCTAATCGAAACTAATGGGGAAACTGGAGAAATTGTCTGGGATAAAGGGCGA
GATTTTGCCACAGTGCGCAAAGTATTGTCCATGCCCCAAGTCAATATTGTCAAGAAAACAGAAGTACAGACAGGCGG
ATTCTCCAAGGAGTCAATTTTACCAAAAAGAAATTCGGACAAGCTTATTGCTCGTAAAAAAGACTGGGATCCAAAAA
AATATGGTGGTTTTGATAGTCCAACGGTAGCTTATTCAGTCCTAGTGGTTGCTAAGGTGGAAAAAGGGAAATCGAAG
AAGTTAAAATCCGTTAAAGAGTTACTAGGGATCACAATTATGGAAAGAAGTTCCTTTGAAAAAAATCCGATTGACTT
TTTAGAAGCTAAAGGATATAAGGAAGTTAAAAAAGACTTAATCATTAAACTACCTAAATATAGTCTTTTTGAGTTAG
AAAACGGTCGTAAACGGATGCTGGCTAGTGCCGGAGAATTACAAAAAGGAAATGAGCTGGCTCTGCCAAGCAAATAT
GTGAATTTTTTATATTTAGCTAGTCATTATGAAAAGTTGAAGGGTAGTCCAGAAGATAACGAACAAAAACAATTGTT
TGTGGAGCAGCATAAGCATTATTTAGATGAGATTATTGAGCAAATCAGTGAATTTTCTAAGCGTGTTATTTTAGCAG
ATGCCAATTTAGATAAAGTTCTTAGTGCATATAACAAACATAGAGACAAACCAATACGTGAACAAGCAGAAAATATT
ATTCATTTATTTACGTTGACGAATCTTGGAGCTCCCGCTGCTTTTAAATATTTTGATACAACAATTGATCGTAAACG
ATATACGTCTACAAAAGAAGTTTTAGATGCCACTCTTATCCATCAATCCATCACTGGTCTTTATGAAACACGCATTG
ATTTGAGTCAGCTAGGAGGTGACTGA
Amino Acid
Cas9
Streptococcus
SEQ ID NO. 32
MDKKYSIGLDIGTNSVGWAVITDEYKVPSKKFKVLGNTDRHSIKKNLIGALLFDSGETAEATRLKRTARRRYTRRKN
RICYLQEIFSNEMAKVDDSFFHRLEESFLVEEDKKHERHPIFGNIVDEVAYHEKYPTIYHLRKKLVDSTDKADLRLI
YLALAHMIKFRGHFLIEGDLNPDNSDVDKLFIQLVQTYNQLFEENPINASGVDAKAILSARLSKSRRLENLIAQLPG
EKKNGLFGNLIALSLGLTPNEKSNEDLAEDAKLQLSKDTYDDDLDNLLAQIGDQYADLFLAAKNLSDAILLSDILRV
NTEITKAPLSASMIKRYDEHHQDLTLLKALVRQQLPEKYKEIFEDQSKNGYAGYIDGGASQEEFYKEIKPILEKMDG
TEELLVKLNREDLLRKQRTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFTVYNELTKVKYVTEGMRKPAF
LSGEQKKAIVDLLEKTNRKVTVKQLKEDYFKKIECEDSVEISGVEDRFNASLGTYHDLLKIIKDKDFLDNEENEDIL
EDIVLTLTLFEDREMIEERLKTYAHLFDDKVMKQLKRRRYTGWGRLSRKLINGIRDKQSGKTILDFLKSDGFANRNF
MQLIHDDSLTFKEDIQKAQVSGQGDSLHEHIANLAGSPATKKGILQTVKVVDELVKVMGRHKPENIVIEMARENQTT
QKGQKNSRERMKRIEEGIKELGSQILKEHPVENTQLQNEKLYLYYLQNGRDMYVDQELDINRLSDYDVDHIVPQSFL
KDDSIDNKVLTRSDKNRGKSDNVPSEEVVKKMKNYWRQLLNAKLITQRKEDNLTKAERGGLSELDKAGFIKRQLVET
RQITKHVAQILDSRMNTKYDENDKLIREVKVITLKSKLVSDFRKDFQFYKVREINNYHHAHDAYLNAVVGTALIKKY
PKLESEFVYGDYKVYDVRKMIAKSEQEIGKATAKYFFYSNIMNFEKTEITLANGEIRKRPLIETNGETGEIVWDKGR
DFATVRKVLSMPQVNIVKKTEVQTGGFSKESILPKRNSDKLIARKKDWDPKKYGGFDSPTVAYSVLVVAKVEKGKSK
KLKSVKELLGITIMERSSFEKNPIDFLEAKGYKEVKKDLIIKLPKYSLFELENGRKRMLASAGELQKGNELALPSKY
VNFLYLASHYEKLKGSPEDNEQKQLFVEQHKHYLDEIIEQISEFSKRVILADANLDKVLSAYNKHRDKPIREQAENI
IHLFTLTNLGAPAAFKYFDTTIDRKRYTSTKEVLDATLIHQSITGLYETRIDLSQLGGD
Amino Acid
Cas9
Staphylococcus
SEQ ID NO. 32
MKRNYILGLDIGITSVGYGIIDYETRDVIDAGVRLFKEANVENNEGRRSKRGARRLKRRRRHRIQRVKKLLFDYNLL
TDHSELSGINPYEARVKGLSQKLSEEEFSAALLHLAKRRGVHNVNEVEEDTGNELSTKEQISRNSKALEEKYVAELQ
LERLKKDGEVRGSINREKTSDYVKEAKQLLKVQKAYHQLDQSFIDTYIDLLETRRTYYEGPGEGSPFGWKDIKEWYE
MLMGHCTYFPEELRSVKYAYNADLYNALNDLNNLVITRDENEKLEYYEKFQIIENVFKQKKKPTLKQIAKEILVNEE
DIKGYRVTSTGKPEFTNLKVYHDIKDITARKEIIENAELLDQIAKILTIYQSSEDIQEELTNLNSELTQEEIEQISN
LKGYTGTHNLSLKAINLILDELWHTNDNQIAIFNRLKLVPKKVDLSQQKEIPTTLVDDFILSPVVKRSFIQSIKVIN
AlIKKYGLPNDIIIELAREKNSKDAQKMINEMQKRNRQTNERIEEIIRTTGKENAKYLIEKIKLHDMQEGKCLYSLE
AIPLEDLLNNPFNYEVDHIIPRSVSEDNSENNKVLVKQEENSKKGNRTPFQYLSSSDSKISYETFKKHILNLAKGKG
RISKTKKEYLLEERDINRFSVQKDFINRNLVDTRYATRGLMNLLRSYFRVNNLDVKVKSINGGFTSFLRRKWKFKKE
RNKGYKHHAEDALIIANADFIFKEWKKLDKAKKVMENQMFEEKQAESMPEIETEQEYKEIFITPHQIKHIKDFKDYK
YSHRVDKKPNRELINDTLYSTRKDDKGNTLIVNNLNGLYDKDNDKLKKLINKSPEKLLMYHHDPQTYQKLKLIMEQY
GDEKNPLYKYYEETGNYLTKYSKKDNGPVIKKIKYYGNKLNAHLDITDDYPNSRNKVVKLSLKPYRFDVYLDNGVYK
FVTVKNLDVIKKENYYEVNSKCYEEAKKLKKISNQAEFIASFYNNDLIKINGELYRVIGVNNDLLNRIEVNMIDITY
REYLENMNDKRPPRIIKTIASKTQSIKKYSTDILGNLYEVKSKKHPQIIKKG
DNA
IRES-TMV
tobacco mosaic virus
SEQ ID NO. 33
GAATTCGTCGATTCGGTTGCAGCATTTAAAGCGGTTGACAACTTTAAAAGAAGGAAAAAGAAGGTTGAAGAAAAGGG
TGTAGTAAGTAAGTATAAGTACAGACCGGAGAAGTACGCCGGTCCTGATTCGTTTAATTTGAAAGAAGAAA
DNA
IRES-NtHSF
Nicotiana tabacum IRES
SEQ ID NO. 34
GGCACGAGGCTCCCATTAATATTTCTTCTTCTGTGTAATTCCATTATTCTGTAGTAGATTCACGTCCGAGTTTAAAG
AAGAGAGAAAACTGAAAAGGCAGAAAATTCCAGAGCTTTAGATTTAGCCAAAGATAGTTATGGTCGTGTTGTTCTTG
GTGAAGATTGGCAAAGTAGGAGCCAATGGAAGAAACTAAGATCATAATCAATCGCCCCAAAAACAACCTTGTTCATT
CTATGGTTTTTCTCTTCGGTTTCTATGTTTGGGATTGGGAATTCCTCACTGTCCTTTTGCTTTTCAGTTATTGCTCC
TTCTAATTTTCCCTAGCTAGGATCTTCTCAATTAATTTCCTTTTTCATTTTCAACTAACTCATAATTAGCCCAAATC
TTCAAAAGAGTTTTGTGTAAGTTGATAGACGTTTAGAGAAACAGAGAAATACAGGGGAAAAACAAGGG
DNA
IRES-TuMV
turnip mosaic potyvirus IRES
SEQ ID NO. 35
GGGAAAGCTTGCATGCCTGCAGGTCGACTCTAGAAAAATATAAAAACTCAACACAACATACACAAAACGATTAAAGC
AAACACAATCTTTCAAAGCATTCAAAGCATTCAAGCAATCAAAGATTTTCAAATCTTTTGTCGTTATCAAAGCAATC
ACCAACAGGATCCAGGATCCCCGGGTGGTCAGTCCCTT
DNA
IRES-TEV
tobacco etch virus IRES
SEQ ID NO. 36
AAATAACAAATCTCAACACAACATATACAAAACAAACGAATCTCAAGCAATCAAGCATTCTACTTCTATTGCAGCAA
TTTAAATCATTTCTTTTAAAGCAAAAGCAATTTTCTGAAAATTTTCACCATTTACGAACGATAGCA
DNA
CITE-SNTV
satellite tobacco necrosis virus CITE
SEQ ID NO. 37
AAAAAAAAAAAAGTAAAGACAGGAAACTTTACTGACTAACTCCCAGAGGTTCACAATGTTAGTGATGGGGCGCTGAA
AGATGCGTAGCTACCCTTCTGGAGCCACTTCCTGGTGGTAAGCAGAAATCCAAGGGTACGGTGGTACGGTGGAAAGC
AGTCCC
NtHSF1_IRES_F primer
Artificial
SEQ ID NO. 37
GGCACGAGGCTCCCATTAATATTTC
TuMV_IRES_F primer
Artificial
SEQ ID NO. 37
GGGAAAGCTTGCATGCCTG
TEV_IRES_F primer
Artificial
SEQ ID NO. 37
GAAATAACAAATCTCAACACAACATATAC
rpoA-for primer
Artificial
SEQ ID NO. 37
GCACCAAAGAAGGCGTTCAG
rpoA-rev primer
Artificial
SEQ ID NO. 37
GGTCAGGTGGCAGATCACAT

1-25. (canceled)

26. A genetically modified bacterium expressing a heterologous nucleotide sequence operably linked to a promoter encoding a heterologous Euk-mRNA wherein said heterologous Euk-mRNA is not translatable in said donor prokaryote and further includes:
at least one untranslated region (UTR) forming a ribosomal regulatory control region configured to facilitate recruitment of eukaryotic ribosomes;
a coding region encoding at least one CRISPR-associated endonuclease that is competent to be translated in a eukaryote;
removal of prokaryote ribosomal binding sites;
a Kozak consensus sequence; and
a poly-adenylated (poly-A) region configured to facilitate Poly-A binding proteins;
co-expressing at least one guide RNA (gRNA) configured to hybridize with a target genome sequence in a recipient eukaryote.

27. The bacterium of claim 26 wherein said coding region encoding at least one CRISPR-associated endonuclease comprises a coding region encoding a Cas9 protein.

28. The bacterium of claim 26 wherein said coding region encoding at least one CRISPR-associated endonuclease comprises a coding region encoding a CRISPR-associated endonuclease selected from the group consisting of: the amino acid sequence according to SEQ ID. NO. 30; the amino acid sequence according to SEQ ID. NO. 32; the amino acid sequence according to SEQ ID. NO. 33, the nucleotide sequence according to SEQ ID. NO. 29; and the nucleotide sequence according to SEQ ID. NO. 31.

29. The bacterium of claim 27 wherein said Euk-mRNA further includes a stabilization region comprising at least two hybridizable sequences positioned at the 5′ and 3′ ends of said Euk-mRNA respectively, which are further configured to form a hairpin loop.

30-32. (canceled)

33. The bacterium of claim 29 and further comprising a Euk-mRNA having a eukaryotic stop signal.

34. The bacterium of claim 29 wherein said untranslated region (UTR) forming a ribosomal regulatory control region comprises a untranslated region (UTR) forming a ribosomal regulatory control region selected from the group consisting of: a Internal Ribosome Entry Sites (IRES) sequence; and a positioned cap independent translation element” (CITE) sequence.

35. The bacterium of claim 34 wherein said IRES sequence comprises an IRES sequence selected from the group consisting of: a tobacco mosaic virus IRES (crTMV); a tobacco etch virus IRES (TEV); a turnip mosaic potyvirus IRES (TuMV); a Nicotiana tabacum heat shock protein IRES (NtHSF) and an artificial IRES sequence.

36. The bacterium of claim 34 wherein said IRES sequence comprises an IRES sequence selected from the group consisting of: the nucleotide sequences according to SEQ ID NOs. 34-37.

37. The bacterium of claim 34 wherein said CITE sequence comprises a CITE sequence selected from the group consisting of: a satellite tobacco necrosis virus (SNTV) CITE; and an artificial CITE sequence.

38. The bacterium of claim 34 wherein said CITE sequence comprises the nucleotide sequences according to SEQ ID NO. 38.

39. The bacterium of claim 29 wherein said Euk-mRNA further comprises at least one additional endogenous 3′ UTR configured to recruit protein complexes that facilitate eukaryote ribosome interaction.

40. (canceled)

41. The bacterium of claim 29 and further comprising the step of generating a genetically modified donor prokaryote co-expressing with said Euk-mRNA and said gRNA a heterologous nucleotide sequence operably linked to a promoter encoding at least one heterologous helper gene encoding at least one helper protein configured to increase transport efficiency of said Euk-mRNAs and said gRNA from donor prokaryotes to recipient eukaryotic cells.

42. The bacterium of claim 41 wherein said at least one helper protein configured to increase transport efficiency of said Euk-mRNAs from donor prokaryotes to recipient eukaryotic cells comprises at least one chimeric RNA binding helper protein coupled with a bacterial secretion signal.

43. The bacterium of claim 42 wherein said at least one chimeric RNA binding helper protein coupled with a bacterial secretion signal is selected from the group consisting of: dsRNA binding protein 2 (DRB4) coupled with a OmpA bacterial secretion signal; and phloem protein 2-A1 (PP2-A1) coupled with a OmpA bacterial secretion signal.

44. The bacterium of claim 43 wherein said at least one chimeric RNA binding helper protein coupled with a bacterial secretion signal is selected from the group consisting of: the amino acid sequence of SEQ ID NO. 11 coupled with the amino acid sequence of SEQ ID NO. 25; and the amino acid sequence of SEQ ID NO. 11 coupled with the amino acid sequence of SEQ ID NO. 27.

45. The bacterium of claim 42 wherein said at least one helper protein configured to increase transport efficiency of said Euk-mRNAs from donor prokaryotes to recipient eukaryotic cells is selected from the group consisting of: the amino acid sequence according to SEQ ID NO. 25; the amino acid sequence according to SEQ ID NO. 27; the nucleotide sequence according to SEQ ID NO. 24; and the nucleotide sequence according to SEQ ID NO. 26.

46. The bacterium of claim 42 wherein said bacterial secretion signal comprises a bacterial secretion signal selected from the group consisting of: PelB (pectate lyase B) from Erwinia carotovora; OmpA (outer-membrane protein A); StII (heat-stable enterotoxin 2); Endoxylanase from Bacillus sp.; PhoA (alkaline phosphatase); OmpF (outer-membrane protein F); PhoE (outer-membrane pore protein E); MalE (maltose-binding protein); OmpC (outer-membrane protein C); Lpp (murein lipoprotein); LamB (λ receptor protein); OmpT (protease VII); LTB (heat-labile enterotoxin subunit B); and HylA (a-Haemolysin).

47. The bacterium of claim 42 wherein said bacterial secretion signal comprises a bacterial secretion signal selected from the group consisting of: an amino acid sequence according to SEQ ID NOs. 11-23.

48. The bacterium of claim 29 wherein said a stabilization region comprising two hybridizable sequences positioned at the 5′ and 3′ ends of said Euk-mRNA respectively, which are further configured to form a Euk-mRNA hairpin loop comprises at least two hybridizable GC-rich sequences positioned at the 5′ and 3′ ends of said Euk-mRNA respectively, which are further configured to form a hairpin Euk-mRNA loop structure.

49. The bacterium of claim 29 wherein said Euk-mRNA loop structure is configured to stabilize the Euk-mRNA molecule, prevent degradation, enhance transport efficiency, and not interfere with eukaryote ribosome binding and translation in said recipient eukaryote.

50-77. (canceled)

78. A genetically modified bacterium expressing a heterologous nucleotide sequence operably linked to a promoter encoding a heterologous Euk-mRNA wherein said heterologous Euk-mRNA is not translatable in said donor prokaryote and further includes:
at least one untranslated region (UTR) forming a ribosomal regulatory control region configured to facilitate recruitment of eukaryotic ribosomes;
a coding region encoding at least one gene-editing endonuclease that is competent to be translated in a eukaryote and configured to target a genome sequence;
removal of prokaryote ribosomal binding sites;
a Kozak consensus sequence;
a poly-adenylated (poly-A) region configured to facilitate Poly-A binding proteins.

79. The bacterium of claim 78 wherein said Euk-mRNA further includes a stabilization region comprising at least two hybridizable sequences positioned at the 5′ and 3′ ends of said Euk-mRNA respectively, which are further configured to form a hairpin loop.

80. The method of claim 78 wherein said coding region encoding at least one CRISPR-associated endonuclease comprises a coding region encoding a Cas9 protein.

81. The bacterium of claim 78 wherein said coding region encoding at least one CRISPR-associated endonuclease comprises a coding region encoding a CRISPR-associated endonuclease selected from the group consisting of: the amino acid sequence according to SEQ ID. NO. 30; the amino acid sequence according to SEQ ID. NO. 32; the amino acid sequence according to SEQ ID. NO. 33, the nucleotide sequence according to SEQ ID. NO. 29; and the nucleotide sequence according to SEQ ID. NO. 31.

82. The bacterium of claim 80 and further comprising the step of co-expressing at least one guide RNA (gRNA) configured to hybridize with said target genome sequence in a recipient eukaryote.

83. The bacterium of claim 78 wherein said gene-editing endonuclease comprises a gene-editing endonuclease selected from the group consisting of: CRISPR-associated endonuclease, Cas9, Cas3, a TALAN-associated endonuclease; a meganuclease, and a zinc-finger associated endonuclease.

84-175. (canceled)

176. A bacterium expressing at least one heterologous nucleotide sequence according to SEQ ID NOs. 1-10, and wherein said protein coding region in said sequence is replaced with a target protein of interest.

177-181. (canceled)

182. The bacterium of claim 176 wherein said target protein of interest is selected from the group consisting of: a CRISPR-associated endonuclease, Cas9, Cas3, a TALAN-associated endonuclease; a meganuclease, and a zinc-finger associated endonuclease
